SEQUENCE LISTING (15 sequences)

SEQ ID NO: 1 - protein, 602 aa, Cupriavidus necator
MDSRITTILERYRSDRTRLIDILWDVQHEYGHIPDAVLPQLGAGLKLSPLDIRETASFYHFFLDKPSGKYRIYLCNSVIAKINGYQAVREALERETGIRFGETDPNGMFGLFDTPCIGLSDQEPAMLIDKVVFTRLRPGKITDIIAQLKQGRSPAEIANPAGLPSQDIAYVDAMVESNVRTKGPVFFRGRTDLRSLLDQCLLLKPEQVIETIVDSRLRGRGGAGFSTGLKWRLCRDAESEQKYVICNADEGEPGTFKDRVLLTRAPKKVFVGMVIAAYAIGCRKGIVYLRGEYFYLKDYLERQLQELREDGLLGRAIGGRAGFDFDIRIQMGAGAYICGDESALIESCEGKRGTPRVKPPFPVQQGYLGKPTSVNNVETFAAVSRIMEEGADWFRAMGTPDSAGTRLLSVAGDCSKPGIYEVEWGVTLNEVLAMVGARDARAVQISGPSGECVSVAKDGERKLAYEDLSCNGAFTIFNCKRDLLEIVRDHMQFFVEESCGICVPCRAGNVDLHRKVEWVIAGKACQKDLDDMVSWGALVRRTSRCGLGATSPKPILTTLEKFPEIYQNKLVRHEGPLLPSFDLDTALGGYEKALKDLEEVTR

SEQ ID NO: 2 - protein, 234 aa, Cupriavidus necator
MSIQITIDGKTLTTEEGRTLVDVAAENGVYIPTLCYLKDKPCLGTCRVCSVKVNGNVAAACTVRVSKGLNVEVNDPELVDMRKALVEFLFAEGNHNCPSCEKSGRCQLQAVGYEVDMMVSRFPYRFPVRVVDHASEKIWLERDRCIFCQRCVEFIRDKASGRKIFSISHRGPESRIEIDAELANAMPPEQVKEAVAICPVGTILEKRVGYDDPIGRRKYEIQSVRARALEGEDK

SEQ ID NO: 3 - protein, 209 aa, Cupriavidus necator
MRAPHKDEIASHELPATPMDPALAANREGKIKVATIGLCGCWGCTLSFLDMDERLLPLLEKVTLLRSSLTDIKRIPERCAIGFVEGGVSSEENIETLEHFRENCDILISVGACAVWGGVPAMRNVFELKDCLAEAYVNSATAVPGAKAVVPFHPDIPRITTKVYPCHEVVKMDYFIPGCPPDGDAIFKVLDDLVNGRPFDLPSSINRYD

SEQ ID NO: 4 - protein, 488 aa, Cupriavidus necator
MSRKLVIDPVTRIEGHGKVVVHLDDDNKVVDAKLHVVEFRGFEKFVQGHPFWEAPMFLQRICGICFVSHHLCGAKALDDMVGVGLKSGIHVTPTAEKMRRLGHYAQMLQSHTTAYFYLIVPEMLFGMDAPPAQRNVLGLIEANPDLVKRVVMLRKWGQEVIKAVFGKKMHGINSVPGGVNNNLSIAERDRFLNGEEGLLSVDQVIDYAQDGLRLFYDFHQKHRAQVDSFADVPALSMCLVGDDDNVDYYHGRLRIIDDDKHIVREFDYHDYLDHFSEAVEEWSYMKFPYLKELGREQGSVRVGPLGRMNVTKSLPTPLAQEALERFHAYTKGRTNNMTLHTNWARAIEILHAAEVVKELLHDPDLQKDQLVLTPPPNAWTGEGVGVVEAPRGTLLHHYRADERGNITFANLVVATTQNNQVMNRTVRSVAEDYLGGHGEITEGMMNAIEVGIRAYDPCLSCATHALGQMPLVVSVFDAAGRLIDERAR

SEQ ID NO: 5 - protein, 165 aa, Cupriavidus necator
MNAPAEFPYVTLADFDDPSTLIYGIGNVGRQDDGLGWAFIDRLEAESLCSGAEVQRHYQLHLEDADLISRKRKVLFIDATKDASVASFSLERAEPRMDFSFTSHAISIPSIMATCQRCFQCLPEVYVLAIRGYEWELRMGLTPQARHNLDDAIAHFSMRAERQTS

SEQ ID NO: 6 - protein, 167 aa, Cupriavidus necator
MKEQEIDRIATMIYEAPLGEYIGRDGAAILAEHAAEARLLKGDEFLYRRGDVTSSFYIVTDGRLALVREKTNERTAPIVHVLEKGDLVGELGFIDQTPHSLSVRALGDAAVLSFSAESIKPLITEHPELIFNFMRAVIKRVHHVVVTVGEHERELQEYISTGGRGRG

SEQ ID NO: 7 - protein, 113 aa, Cupriavidus necator
MHEMSLAVGVLQIVEDVAQRDGFSRVTAVRLEIGRLSSIEPEALRFCFEEVVRGSVADGARLEIVDTPGAGWCLHCSETVAIGALYDPCPQCGGYQVQPTGGTEMRVMDLEVA

SEQ ID NO: 8 - protein, 320 aa, Cupriavidus necator
MCTNCGCAAGETRIEGQELEHVDEHAHADGTVHGHAPHHHGEHDHDHGPAYRAVRGTDHLHYGHGPAGAHAPGMSQARMVKIEQDILGKNNAYAAQNRRWFDEHGVFALNFVSSPGSGKTTLLVRTIEALKSTSRLAVIEGDQQTSFDAERIRATGVQALQINTGKGCHLDAHMVGHALEKLRPEDESVLLIENVGNLVCPSAFDLGEAHKVVILSVTEGEDKPLKYPDMFRAASLMLLNKCDLLPHLSFDVERAIEYAKRVNPDLHVIRTSSATGEGFDAWLTWIADGLAGQAARRSQSMELLRSRIAGLEAQLAALKV

SEQ ID NO: 9 - protein, 807 aa, Cupriavidus necator
MLMPRRPRNPRTVRIRIRVRGVVQGVGFRPFVYRLARELGLAGWVRNDGAGVDIEAQGSAAALVELRERLRRDAPPLARVDEIGEERCAAQVDADGFAILESSRSDAAVHTAIGHDTAVCPDCLAELFDPANRRYRYAFINCTQCGPRYTLTWALPYDRATTSMAPFPQCRPCLDEYNAPEHRRFHAEPNACPDCGPSLALLNAQGMPVEDVDPIAETVARLQRGEIVAIKGLGGFHLACDAHNADAVARLRSRKQREEKPFAVMVANLATAAQWGDIGSGEAALLTASERPIVLLRKRSGVDGRFAGVAPGLVWLGVMLPYTPLQYLLFHEAAGRPEGLGWLAQPQSLVLVMTSANPGGEPLVTGNDEAAQRLTGIADAFLLHDREILVRCDDSVVRGDGEPAPHVQFIRRARGYTPRAIKLARSGPSVLALGGSFKNTVCLTRGDEAFVSQHVGDLGNAATCEALIEAVAHLQRVLEIRPQLVAHDLHPDFFSTRHAAELAAQWGVPAVAVQHHHAHIAAVLAEHGSDEPAIGLALDGVGLGDDGQAWGGELLLVDGGACKRLGHLRELPLPGGDRAAREPWRMAAAALHAMGRGEEIEGRFPRQPGAPMVNRMLAQRLNAPLSSSMGRWFDAAAGLLGTRETMAYEGQAAMLLEGLAESWGEQPSPGRPKTVAHSLGGVPRSGGGTYKALALPDAWRIDAGNTLDLLPLLEALSAETNAARGAAQFHATLVAALEAWTVATVQVTGVRTVVFGGGCFLNHILARNLCRRLAARGLTVLTARQLPPNDGGIALGQVWVALQRAPN

SEQ ID NO: 10 - protein, 90 aa, Cupriavidus necator
MCLAIPARLVELQADQQGVVDLSGVRKTISLALMADAVVGDYVIVHVGYAIGKIDPEEAERTLRLFAELERVQPPASEPMHGMNIHQEPA

SEQ ID NO: 11 - protein, 379 aa, Cupriavidus necator
MKYIEEFRDGELAQRIAAHVRAEARPGQRYNFMEFCGGHTHAISRYGVTELLPENVRMIHGPGCPVCVLPIGRIDLALHLALERDAIVCTYGDTMRVPASGGMSLIRAKAHGADIRMVYSAADALKIAQRHPQREVVFLAIGFETTTPPTALIIREAKARQVDNFSVLCCHVLTPSAITHILESPEVRDYGTVPIDGFVGPAHVSIVIGTRPYEHFSREYGKPVVIAGFEPLDVMQAILMLVRQVNSGRAEVENEFVRAVTRDGNESAQAMVSEVFELRPSFEWRGLGEVPYSALRIRAQFARFDAEQRFDLRYRPVPDNKACECGAILRGVKKPTDCKLFATVCTPENPMGSCMVSSEGACAAHYSYGRFKDIPLVAA

SEQ ID NO: 12 - protein, 351 aa, Cupriavidus necator
MSGTVKLGYQRPLNIKSGRIDMGHGAGGRAAAQLIQELFVAAFDNEWLRQGNDQAAFAMPAGARMVMATDAHVVSPLFFPGGDIGSLSVHGTINDVAMAGAKPLYLAASFILEEGFPLADLKRIVESMAGAAREAGVPIVTGDTKVVEQGKGDGVFITTTGVGVVPAGILIDGAGARPGDAILLSGTMGEHGVAILSKRESLEFDTEIRSDSAALHDLVAQMLAVVPGVRVLRDPTRGGLATTLNEISSQSGVGMVLDEAAIPVLPQVDAACELLGLDPLYVANEGKLVAICAAADADALLAAMRGHPLGREARRIGEVIEDGRHFVQMRTKFGGMRVVDWLSGEQLPRIC

SEQ ID NO: 13 - protein, 597 aa, Cupriavidus necator
MRILLLTHSFNSLTQRLFVELRQRGHLVSVEFDIADSVTEEAVALFAPDLVIAPFLKRAIPERIWSRLVCLVVHPGIVGDRGPSALDWAIVRDERSWGVTVLQANGEMDAGPVWASATFPMRAARKSSLYRNEVTVAAVQAVLEALAAFEAGWRSANDAQSGPGTWNPAMRQAERGIDWARESTAAVLAKLHAADSFPGVPDALFGQPCRLFDAHAATPQTVARAPRGQPGDIVARREHAVLRLTVDAGVWIGHAKLAVRDEWENTSLPSLKLPVAQVFHEAWAQLPDLSLPLEHDAGEWSEFRYWEDDSGAGLVGYLAFDCYNGAMSTAQCQRLCAALRYARGRQTRVLVLLGGEDFFSNGIHLHQIEAAEHRGAESAADASWRNIQAMDDVALEILAFSDRMTVSALRGNAGAGGVFLALAADQVWAREGVLVNPHYKNMGNLYGSEYWTYLLPQRVGAQRASDLMDGRLPMSVRRAIEIGLIDASLDGDARSCLAEIGRRAVALARAPDYAAHIDNKRRKRAADEAAKPLAQYREEELVHMHRNFYGFDPSYHVARYHFVYKLPHAHTPRHLAMHRGGSLPAAQELQALAGKRS

SEQ ID NO: 14 - DNA, 181 bp, Synthetic construct
  source 1..181: mol_type = other DNA
cattattatcatgacattaacctataaaaataggcgtatcacgaggccctttcgtcttcacctcgagaaatcataaaaaatttatttgctttgtgagcggataacaattataatagattcaattgtgagcggataacaatttcacacagaattcattaaaggagaaattaactatgagagg

SEQ ID NO: 15 - DNA, 181 bp, Synthetic construct
  source 1..181: mol_type = other DNA
cattattatcatgacatgaattcagctcgacaacgcaagcggccgccggaaaggaacatcaggtaccaaatcataaaaaatttatttgctttgtgagcggataacaattataatagattcaattgtgagcggataacaatttcacacaatgcaaattaaaggagaaattaactatgagagg

(19) United States
(12) Patent Application Publication
(10) Pub. No.: US 2026/0176639 A1
(43) Pub. Date: Jun. 25, 2026

(54) HOST CELL WITH INCREASED CELLULAR REDUCING POWER FROM A HETEROLOGOUS HYDROGENASE

(71) Applicant: THE REGENTS OF THE UNIVERSITY OF CALIFORNIA, Oakland, CA (US)

(72) Inventors: Justin PANICH, Berkeley, CA (US); Robert BERTRAND, Oakland, CA (US); Steven W. SINGER, Oakland, CA (US); Jay D. KEASLING, Berkeley, CA (US)

(21) Appl. No.: 19/334,626

(22) Filed: Sep. 19, 2025

Related U.S. Application Data
(63) Continuation of application No. PCT/US2024/020959, filed on Mar. 21, 2024.
(60) Provisional application No. 63/491,502, filed on Mar. 21, 2023.

(51) Int. Cl. C12N 15/70; C12N 9/02; C12N 9/04; C12N 9/12; C12N 15/52

(52) U.S. Cl. CPC C12N 15/70; C12N 9/0006; C12N 9/0008; C12N 9/0067; C12N 9/0093; C12N 9/1241; C12N 15/52; C12Y 101/01244; C12Y 102/01046; C12Y 117/01; C12N 2830/34

(57) ABSTRACT

The present invention provides a genetically modified host cell comprising a heterologous hydrogenase, or dehydrogenase, or homologous variant thereof, wherein the genetically modified host cell has an increased cellular reducing power compared an unmodified host cell. In some embodiments, the hydrogenase is a soluble hydrogenase (SH) from Cupriavidus necator H16, or a homologous variant thereof.

### CROSS-REFERENCE TO RELATED APPLICATIONS

This application claims priority as a continuation application to PCT International Patent Application No. PCT/US2024/020959, filed Mar. 21, 2024, which claims the priority benefit of U.S. Provisional Application Nos. 63/491,502, filed Mar. 21, 2023; all of which are hereby incorporated by reference in their entireties.

### STATEMENT OF GOVERNMENTAL SUPPORT

This invention was made with government support under Contract No. DE-AC02-05CH11231 awarded by the United States Department of Energy. The government has certain rights in the invention.

### REFERENCE TO SEQUENCE LISTING

The instant application contains a Sequence Listing which has been submitted electronically in XML format and is hereby incorporated by reference in its entirety. Said XML copy, created on Mar. 3, 2026, is named “2022-146-03 Sequence Listing.XML” and is 18,858 bytes in size.

### FIELD OF THE INVENTION

This invention relates generally to a host cell with increased cellular reducing power from a heterologous hydrogenase.

### BACKGROUND OF THE INVENTION

Microorganisms potentiate a paradigm shift in industrial production (Zhang et al. 2017). Enzymes, Nature's most powerful tool for enantioselective biosynthesis, can make commodities ranging from fuels to pharmaceuticals (Kung et al. 2012; Pearsall et al. 2015). Biomanufacturing greatly eliminates the often-enormous heat, pressure, and toxicity requirements in chemical production while decreasing the lifetime environmental impact of those products (Adom et al. 2014). Though biomanufacturing is the key for a sustainable future, bioproduction often collides with a harsh economic reality (Levi & Cullen, 2018; Moore, 2008a; 2008b; Tollefson, 2008). Every industrial bioprocess seeks to maximize titer, rate, and yield (TRY). However, this goal is often frustrated by limitations in the supply of ATP and NAD(P)H needed transform substrates into desired products and to keep the host alive (Wang et al. 2013; Thakker et al. 2015). For example, terpenoids need two NADPH and three ATP per isopentenyl monomer when generated through the mevalonate intermediate, and even stationary phase bacteria consume about 200,000 ATP per second per cell (Deng et al. 2021). This energy must be provided by sacrificing much of the feedstock to phosphorylation, oxidation and respiration. Consequently, bioprocesses often fall dismally short of their maximum theoretical yields. This problem is especially true of biofuels as they are electron-rich by design (Cruz-Morales et al. 2022). As feedstock constitutes the majority of the operating cost in bioindustry, this split-usage of feedstock as both energy and substrate provides a seemingly intractable problem that renders industrial microbiology unsuitable for generating anything other than designer specialties.

Various strategies are used to resolve this substrate/energy dichotomy. The energetics of life are not optimized, and there are enormous variations in how lifeforms resolve matters as fundamental as pathway flux, cofactor supply, and protein cost (Bar-Even et al. 2012; Flamholz et al. 2013; Noor et al. 2014; Du et al. 2018). Nature therefore provides a rich toolbox for building artificially constructed pathways with improved bioenergetics (Yu et al. 2018a; Bogorad et al. 2014; Ng et al. 2015; Clomburg et al. 2019; Yu & Liao, 2018; Yang et al. 2016; Kang et al. 2016). An alternative approach is to pair redox-surplus with redox-deficient pathways within the same host (Guo et al. 2019; Dittrich et al. 2009). Enabling CO₂ fixation into hydrocarbons, carbohydrates, and biomass via a sacrificial substrate, or oxidizing fermented molecules into CO₂ and NADH, could both be considered variations of this core concept (Bang & Lee, 2018; Antonovsky et al. 2016; Guadalupe-Medina et al. 2013; Balzer et al. 2013; Zhuang & Li, 2013). Retaining substrate by rewiring metabolic processes to avoid otherwise obligatory decarboxylative events is an elegant strategy, and examples include non-oxidative glycolysis and the modified serine cycle (Lin et al. 2018; Tashiro et al. 2015; Meadows et al. 2016; Yu et al. 2018; Yu & Liao, 2018).

Conceptually, the simplest solution is to give the host more electrons. Providing an external source of NADH would allow the microorganism to apply reductions to biochemical pathways, generate ATP by respiration, or perform a hydride or phosphate exchange reaction to create NADPH using redox-balancing enzymes PntAB and NADK. Importantly, owing to the numerous mechanisms that tie the concentration of metabolic intermediates and cofactors to carbon flux (Millard et al. 2021; Litsios et al. 2018; Holm et al. 2010), feeding NADH to a microorganism would obviate the use of feedstock as a source of cellular energy and divert it away from decarboxylative fates. The carbon that is liberated by surplus NADH would then be free to act as extra substrate for bioconversion, resulting in an improved yield for a target molecule. Such a model has neither been perceived or adopted by either industry or the literature. For example, the few examples available regarding cofactor feeding have sought to drive specific reactions in vitro or in vivo that use NAD(P)H as reductant (e.g., see Lonsdale et al. 2015; Al-Shameri et al. 2020). There is broad interest in assimilating reduced C₁ compounds such as methane, methanol, carbon monoxide and formic acid into value-added chemicals using methylotrophic chemistry, and of valorizing CO₂ using H₂ as electron donor and chemolithoautotrophic chemistry (Martín et al. 2015; Liu et al. 2016; Nybo et al. 2015; Dürre, 2017; Nangle et al. 2020). However, there is no demonstration that bioreactors fed with carbohydrates (e.g., glucose, acetate) can also be fed with supplemental reducing power as a generalizable strategy for enhancing the cost-effectiveness of industrial microbiology.

### SUMMARY OF THE INVENTION

The present invention provides for a system for introducing a nucleic acid encoding a hydrogenase, and/or a dehydrogenase, into a host cell comprising: (a) an expression vector comprising hydrogenase genes, and/or dehydrogenase genes, operatively linked to a promoter; and (b) a delivery vector encoding genes to integrate the hydrogenase genes, and/or dehydrogenase genes, and the promoter into a chromosome of the host cell.

In some embodiments, the hydrogenase is any hydrogenase, such as any wild-type hydrogenase. In some embodiments, the hydrogenase is Cupriavidus necator H16 hydrogenase. In some embodiments, the dehydrogenase is any dehydrogenase, such as formate dehydrogenase, methanol dehydrogenase, formaldehyde dehydrogenase, or any wild-type dehydrogenase thereof.

In some embodiments, the genes to integrate the hydrogenase genes and the promoter into a chromosome of the host cell are the genes of the Cre-Lox or Flp-FRT systems, or any recombinases described in Table 1.

The present invention provides for a nucleic acid comprising: (a) one or more nucleotide sequence(s) encoding a hydrogenase, dehydrogenase, or homologous thereof, each independently operatively linked to a promoter; and (b) optionally a further nucleotide sequence encoding a NAD(P) transhydrogenase operatively linked to a promoter; and (c) optionally a further nucleotide sequence encoding a nucleotide sequence of interest operatively linked to a promoter.

The present invention provides for a nucleic acid comprising: (a) a first nucleotide sequence encoding Cupriavidus necator H16 HoxF, HoxU, HoxY, HoxH, HoxW, and HoxI genes operatively linked to a first promoter; (b) a second nucleotide sequence encoding the gene products of Cupriavidus necator H16 HypA2, HypB2, HypD1, HypE1, HypF2, and HypX genes operatively linked to a second promoter; (c) optionally a third nucleotide sequence encoding a NAD(P) transhydrogenase operatively linked to a third promoter; and (d) optionally a fourth nucleotide sequence encoding a nucleotide sequence of interest operatively linked to a fourth promoter.

NAD(P) transhydrogenase catalyzes the regeneration of NADH. In some embodiments, the NAD(P) transhydrogenase is E. coli NAD(P) transhydrogenase, or a homologous variant(s) thereof.

In some embodiments, the enzyme(s) capable of regenerating NADPH is E. coli PntAB. In some embodiments, the nucleotide sequence of interest comprises one or more biosynthetic genes encoding one or more biosynthetic enzymes for producing a compound of interest. In some embodiments, the one or more biosynthetic enzymes are not biosynthetic enzymes that require NAD(P)H. In some embodiments, the nucleic acid is a vector. In some embodiments, the vector is an expression vector.

The present invention provides for a genetically modified host cell comprising the nucleic acid of the present invention, wherein the genetically modified host cell has an increased cellular reducing power compared an unmodified host cell.

In some embodiments, the hydrogenase is heterologous to the host cell. In some embodiments, the hydrogenase is a soluble hydrogenase (SH) from Cupriavidus necator H16, or a homologous variant thereof. In some embodiments, the nucleic acid or genes encoding the hydrogenase is a 13 gene operon encoding the SH, or homologous variants thereof, is integrated into the genome of the genetically modified host cell.

The present invention provides for a library of individual vectors comprising every permutation of at least four antibiotic selection markers, every permutation of at least four origins of replications, a set of genes encoding a hydrogenase, or dehydrogenase, and optionally a cloning site comprising two unique restriction sites that result in two sticky ends that are not compatible for annealing to each other; wherein each individual vector comprises an antibiotic selection marker, and an origin of replication. In some embodiments, the set of genes comprising 13 genes encoding for a Cupriavidus necator H16 hydrogenase.

The present invention provides for a method for introducing a nucleic acid encoding a heterologous hydrogenase into a host cell comprising: (a) introducing the nucleic acid of the present invention into a host cell, and (b) optionally integrating the nucleic acid into a chromosome of the host cell.

The present invention provides for a method for producing a compound of interest, comprising: (a) providing the host cell of the present invention; (b) increasing reducing power for the host cell; and (c) growing or culturing the host cell in a medium such that the hydrogenase and biosynthetic enzyme(s) are expressed thereby the compound of interest is produced.

In some embodiments, increasing reducing power for the host cell comprises providing or introducing or adding a reducing agent to the medium. In some embodiments, reducing agent is an electron, hydrogen, formate, methanol, formaldehyde, and/or any compound that can donate electron via a dehydrogenase, or a mixture thereof. In some embodiments, the reducing agent is a hydrogen, and the host cell expresses a hydrogenase. In some embodiments, the reducing agent is a formate, and the host cell expresses a formate dehydrogenase. In some embodiments, the reducing agent is a methanol, and the host cell expresses a methanol dehydrogenase. In some embodiments, the reducing agent is a formaldehyde, and the host cell expresses a formaldehyde dehydrogenase. In some embodiments, any source of electrons can be used.

Any source of electrons can be used. For example, photosynthetic cyanobacteria offer the means of harvesting light, the cheapest and most abundant energy source in the universe, to transform CO₂ into specialty chemicals. Alternatively, electric currents can be applied to electroactive bacteria (Yamada et al. 2022; Zheng et al. 2020). Both routes involve the activation of electrons (e⁻) to reduce NAD(P)⁺ into NAD(P)H with the contribution of a proton. Numerous chemical donors can also be used. On balance of various considerations such as cost, reduction potential, aqueous solubility, membrane permeability, toxicity, and availability of enzymatic catalysts, the two best choices for industrial application are formate (CO₂H) and hydrogen gas (H₂) (Partipilo et al. 2023; Claassens et al. 2018). Formate is readily oxidized by NAD⁺- or NADP⁺-specific formate dehydrogenases (FDH) to regenerate NAD(P)H, and numerous FDH are available to satisfy kinetic and thermostability requirements. Formate is highly soluble, non-flammable, membrane permeable, and readily made electrochemically from CO₂. However, it is mildly toxic at high concentrations and requires buffering when supplied as formic acid (Yishai et al. 2016; Cotton et al. 2020). In contrast, H₂ can be oxidized to regenerate NADH via soluble hydrogenases, yielding H⁺ as sole byproduct. As the H⁺ is consumed once NADH is reoxidized, H₂ offers pH neutrality and total atom economy. Moreover, H₂ is non-toxic, exceptionally cheap, contains unparalleled chemical energy per unit mass, and can be produced by over a dozen ‘clean’ and ‘dirty’ production techniques. However, H₂ is poorly soluble, highly flammable, and there is a lack of tools enabling aerobic H₂ consumption beyond chemolithoautotrophic hosts (Lauterbach & Lenz, 2019; Ji & Wang, 2019). Other chemical donors beyond H₂ and formate are also possible, for example, methanol (which can be oxidized to regenerate formaldehyde and NAD(P)H via methanol dehydrogenase) and formaldehyde (which can be oxidized to formic acid and NAD(P)H via formaldehyde dehydrogenase).

The most important chemical donors of electrons are hydrogen gas (H₂) and formic acid (COOH). Less common chemical donors could include methanol and formaldehyde. Electrons can also be donated directly. This can be achieved using photons (photosynthesis) or by applying electrical currents (electricity). These reactions are shown in the following reaction formulae:

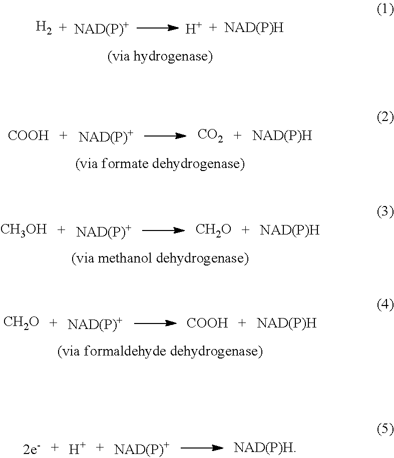

In some embodiments, the compound of interest is a compound naturally produced by the host cell. In some embodiments, the compound of interest is a compound not naturally produced by the host cell. In some embodiments, the compound of interest is a biofuel or bioproduct, or any other organic compound, and the corresponding biosynthetic enzyme(s) for producing the compound of interest thereof, are described and taught in U.S. Pat. Nos. 7,985,567; 8,420,833; 8,852,902; 9,109,175; 9,200,298; 9,334,514; 9,376,691; 9,382,553; 9,631,210; 9,951,345; 10,167,488; 10,273,605; 10,814,724; and 11,660,961; and PCT International Patent Application Nos. PCT/US2014/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/US2011/053787, PCT/US2011/058660, PCT/US2011/059784, PCT/US2011/061900, PCT/US2012/031025, and PCT/US2013/074214 (all of which are incorporated in their entireties by reference). In some embodiments, the compound of interest is a terpene, isoprenoid, carboxylic acid, lactone, trimethylpentanoic acid, 1-deoxyxylulose 5-phosphate, 1-deoxy-D-xylulose 5-phosphate (DXP), fatty acid, or derivatives thereof, alkyl lactone, lactam, isoprenyl alkanoate, 3-methyl-2-buten-1-ol, 3-methyl-3-buten-1-ol, 3-methyl-butan-1-ol, fatty acid ester, alpha-olefin, diacid, diamine, sesquiterpene, bisabolene, or oxidized aromatic amino acid. In some embodiments, the compound of interest is any product or intermediate in the mevalonate (MVA) pathway, including any compound from acetyl-CoA to mevalonate. In some embodiments, the biosynthetic enzyme(s) are phosphomevalonate decarboxylase (PMD), phosphatase, AtoB, hydroxymethylglutaryl-CoA synthase (HMGS), hydroxymethylglutaryl-CoA reductase (HMGR), and/or mevalonate kinase (MK). In some embodiments, the biosynthetic enzyme(s) is a polyketide synthase. In some embodiments, the biosynthetic enzyme(s) is flaviolin polyketide synthase, and the compound of interest is flaviolin.

The present invention provides for a cell-free extract obtained from expression of a heterogenous hydrogenase, or dehydrogenase, expressed in a host cell. In some embodiments, the cell-free extract is in vitro use of the hydrogenase or dehydrogenase. The present invention provides for a method for expressing the hydrogenase, or dehydrogenase, in a host cell, breaking up or lysing the host cell to release contents of cell to produce the cell-free extract.

In some embodiments, the hydrogenase, or dehydrogenase, is heterologous to the host cell. In some embodiments, the genetically modified host cell has an increased cellular reducing power compared an unmodified host cell. In some embodiments, the dehydrogenase is a formate dehydrogenase, methanol dehydrogenase, or formaldehyde dehydrogenase.

In some embodiments, the hydrogenase is a soluble hydrogenase from Cupriavidus necator H16 (also known as Ralstonia eutropha) (as described in Lonsdale et al., 2015), or homologous variants, thereof. In some embodiments, the 13 gene operon encoding the soluble hydrogenase, or homologous variants thereof, is assembled onto one or more, such as two, plasmids. In some embodiments, one plasmid is an expression vector, while the other is a delivery vector allowing for chromosomal integration using a suitable site-specific recombination system, such as Cre-Lox recombination. In some embodiments, one or more, or all, of the genes are codon optimized for the host cell. In some embodiments, the 13 gene operon encoding the SH, or homologous variants thereof, is integrated unto the genome of the genetically modified host cell.

In some embodiments, the genetically modified host cell comprises a nucleic acid encoding the heterologous hydrogenase operably linked to one or more promoters. The genetically modified host cell can comprise one of the plasmids described herein.

The soluble hydrogenase (SH) from Cupriavidus necator H16 has been studied widely and extensively for its ability to convert external hydrogen gas to cellular reducing power in the form of NADH. This enzyme is also remarkable for its tolerance to oxygen, allowing for its use to supplement aerobic metabolisms. Herein is described the assembly/construction of the entire 13 gene operon onto two plasmids. One plasmid is an expression vector, while the other is a delivery vector allowing for chromosomal integration using Cre/lox recombination. We have utilized proteomics, functional analysis, and bioproduct output to establish the functionality of our constructs.

In some embodiments, genetically modified host cell has properties and/or capabilities about equal to or better than the properties described herein, or within a range of any two values described herein.

Described herein are expression platforms and functionality for a heterologous hydrogenase for its use in a host cell, such as E. coli. This is a 13-gene system that allows for the uptake of H₂ from the external environment and concomitant conversion of NAD+ to NADH. While this enzyme has been thoroughly studied and expressed in biological systems, simple and useful expression systems are lacking, and often fall short of displaying the required activity for its utilization for biotechnological applications. Herein is described a single plasmid that is capable of expressing all 13 genes efficiently, along with a strain of E. coli with the 13 gene system integrated on the genome. One of the novel findings disclosed here is that expression of soluble hydrogenase naturally increases NADPH levels, as a consequence of increased NADH. This is of high importance because anabolic pathways for bioproduct synthesis typically require increased NADPH. To our knowledge, this is the first demonstration of increased product output by the expression of soluble hydrogenase in E. coli.

E. coli is easier to manipulate and use for industrial scale bio production than its native host, C. necator H16. It is also disadvantageous to use C. necator for industrial purposes relative to our system because C. necator is extremely limited in product titers and quantities relative to E. coli. Further, our system could be used either aerobically or during anaerobic fermentation, making our system the most flexible for these purposes. While the cost of hydrogen is still prohibitive, the cost is rapidly decreasing, which makes our system attractive.

The present invention provides for a method for producing a biological synthesized compound comprising: (a) optionally genetically modifying a host cell to produce a genetically modified host cell of the present invention, (b) growing or culturing the genetically modified host cell, such as in media and/or conditions described herein, to produce a biological synthesized compound, and (c) optionally recovering the biological synthesized compound produced from the host cell. In some embodiments, the genetically modified host cell is engineered to produce the biological synthesized compound.

In some embodiments, hydrogen (H₂, molecular hydrogen, or hydrogen gas) is provided or introduced into the media. In some embodiments, the hydrogen is part of syngas. In some embodiments, syngas is provided or introduced into the media.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1: Creation and prototyping of hydrogenases for industrial microbiology. (A) H₂ consumption assay of BW25113 E. coli with or without C. necator hydrogenase (“HYD”) cultivated in 20% H₂ in air or 20% N₂ in air; (B) Headspace pressure readings of these same cultures; (C) In vitro specific activity assay of C. necator hydrogenase expressed and extracted from BW25113 E. coli, as compared to C. necator hydrogenase from C. necator; (D) Protein quantification of the 13 C. necator hydrogenase proteins when expressed in BW25113 E. coli, in comparison to protein loads in native organism; (E) The 16-member plasmid library, each containing all 13 C. necator hydrogenase proteins.

FIG. 2: Assessment of effects of H₂ upon malonyl-CoA biosensor flaviolin in BW25113 E. coli when expressing hydrogenase and flaviolin-producing enzyme RppA. (A) Flaviolin is produced by type III PKS RppA via condensation of five units of malonyl-CoA. The unstable intermediate, 2,4,6,8-tetrahydroxy-naphthalene, is spontaneously oxidized by molecular O₂ into flaviolin; (B) The application of a 20% H₂ in air solution enhanced flaviolin titer (A300) by 75% without increasing biomass formation; (C) Photograph of representative result (n=6); untransformed E. coli is shown for contrast.

FIG. 3: Consequences of cofactor feeding on the bioenergetics of life. (A) Quantitative discussions on bioenergy are possible by expressing energy carriers in terms of proton motive force (PMF) equivalencies (molar ratios). Note that hydride exchange from NADH to NADP⁺ by E. coli enzyme PntAB requires proton translocation (Sauer et al. 2004), the H⁺/ATP ratio in E. coli is 4.0 (Steigmiller et al. 2008), and molar yield of hydride transfer from H₂ and formate is >99% (Lauterbach & Lenz, 2013) (B) Cofactor feeding can replace feedstock as the source of bioenergy. When using ideal feedstocks such as glucose, the complete oxidation of one acetyl-CoA yields three NADH, one FADH₂, and one ATP/GTP—equivalent to 40 H⁺ PMF. When using non-ideal feedstocks such as acetate, costs for membrane translocation and activation into acetyl-CoA must be considered. (C). Feedstock diverted from decarboxylative fates can be used to make additional molecules of interest. Mevalonate biosynthesis requires three acetyl-CoA (3×40 H⁺ PMF) and two NADPH (2×11 H⁺ PMF), or 142 H⁺ PMF. Therefore, additional mevalonate can be created by injecting at least 14.2 H₂ or formate into the host. (D). External reducing power can be voided by fermentation. The molar ratios of H₂ or formate required to stimulate common fermentation molecules are shown. (E). Cellular replication requires the expenditure of bioenergy. Each mole of H₂ or formate can also be used to power the formation of 10 g of biomass.

FIG. 4: Titrating effects of H₂ upon acetate consumption by BW25113 E. coli and its attendant effects on CO₂ production when expressing hydrogenase. (A) Scale of experiment; (B) Relative biomass formation when E. coli is fed with various amounts of H₂; (C) Acetate remaining within broth at the conclusion of overnight incubation; (D) The amount of CO₂ observed at the conclusion of an overnight incubation (E) The specific amount of acetate retained by H₂ can be calculated by subtracting acetate within the 0 μmol triplicate from all other triplicates. These values can then be correlated with the amount of H₂ consumed to reveal how much H₂ is experimentally required to prevent the oxidation of acetate. (E) The amount of CO₂ that is no longer produced by H₂ can also be calculated by subtracting all triplicates from the 0 μmol triplicate. Plotting these values versus H₂ consumption reveals how much H₂ is required to prevent the formation of CO₂, mole for mole; (F) By stoichiometry, preventing the oxidation of one mole of acetate should also prevent the formation of two moles of CO₂. Plotting moles of CO₂ avoided versus moles of acetate retained therefore indicates whether such stoichiometry is obeyed.

FIG. 5: Titrating effects of formate upon acetate consumption by BW25113 E. coli and its attendant effects on CO₂ production when expressing formate dehydrogenase. (A) Relative biomass formation when E. coli is fed with various amounts of formate; (B) Acetate remaining within broth at the conclusion of overnight incubation; (C) The amount of CO₂ observed at the conclusion of an overnight incubation (D) As the oxidation of formate yields stoichiometric amounts of CO₂, subtracting moles of formate from total CO₂ determines CO₂ generation from all other types of metabolism. (E) The number of moles of acetate that is retained by formate can be calculated by subtracting acetate within the 0 μmol triplicate from all other triplicates. These values can then be correlated with the amount of formate consumed to reveal how much formate is experimentally required to prevent the oxidation of acetate. (F) The amount of CO₂ that is no longer produced as a consequence of formate feeding (aside from CO₂ resulting from the oxidation of formate itself) can also be calculated by subtracting all triplicates from the 0 μmol triplicate. Plotting these values versus formate consumption reveals how much formate is required to prevent the formation of CO₂, mole for mole; (G) By stoichiometry, preventing the oxidation of one mole of acetate should also prevent the formation of two moles of CO₂. Plotting moles of CO₂ avoided versus moles of acetate retained therefore indicates whether such stoichiometry is obeyed.

FIG. 6: Time-course experiment evaluating acetate retention by H₂ in BW25113 E. coli when expressing hydrogenase. (A) Biomass (B) H₂ remaining (C) CO₂ observed (D) Acetate remaining. This was created through technical replicate cultures (n=3) provided with either 20 ml of H₂ in air or 20 ml of N₂ in air, and assayed at pre-set times. No gas data are recorded between 0 to 4 h because serum bottles were plugged with stoppers at 4 h.

FIG. 7: Assay of effects of formate treatment upon biomass formation, mevalonate production, and fermentation in BW25113 E. coli (Top row) and CM15 E. coli (Bottom row), when grown in 5 g/L of glucose minimal media overnight and when simultaneously expressing formate dehydrogenase and biosynthetic enzymes required for mevalonate biosynthesis. Total fermentation was evaluated by observing all formate, ethanol, acetate, glycerol, lactate, and succinate within broth, then adding all carbon atoms together. No glucose remained in media (not shown). Each experiment was performed in triplicate. Red numbers indicate the calculated percentage of formate used to produce the indicated metabolic effect.

FIG. 8: Assay of effects of H₂ treatment upon biomass formation, mevalonate production, and fermentation in BW25113 E. coli (Top row) and CM15 E. coli (Bottom row), when grown in 5 g/L of glucose minimal media overnight and when simultaneously expressing hydrogenase and biosynthetic enzymes required for mevalonate biosynthesis. Total fermentation was evaluated by observing all formate, ethanol, acetate, glycerol, lactate, and succinate within broth, then adding all carbon atoms together. No glucose remained in media (not shown). Each experiment was performed in triplicate. Red numbers indicate the calculated percentage of H₂ used to produce the indicated metabolic effect.

FIG. 9: Construction of the first 13-gene hydrogenase plasmid (HYD|Kan:P15A). The original plasmid encoding the complete hydrogenase plasmid was constructed using plasmid components pQE80L-SH and pSU-HypA2-X, kindly donated by Sargent and Lamont (2017). The broad scheme was to amplify the entire hydrogenase operon encoded on pQE80L-SH and ligate it into pSU-HypA2-X non-directionally through its unique EcoRI restriction site. The hydrogenase heterotetramer (hoxF, hoxU, hoxY, hoxH), along with two auxiliary genes (hoxW and hoxI), comprises the first operon, under control of the IPTG-inducible T5 promoter. The remaining auxiliary genes (hypA2, hypB2, hypD1, hypE1, hypF2, hypX) comprise the second operon, under control of the constitutive TatA promoter. To facilitate this strategy, it was first necessary to include the T5 promoter of pQE80L-SH within the insertion fragment by moving an EcoRI site on pQE80L-SH about 150 bp upstream from its original location. This was achieved using overlapping primers. To enable additional customization by the end user, a novel cloning site composed of NotI and KpnI restriction sites was also incorporated. Ligation of modified pQE80L-SH template into pSU-HypA2-X yields HYD(KAN|P15A). The hydrogenase plasmid contains a kanamycin selection marker and the P15A origin of replication, which are both inherited from the pSU-HypA2-X backbone. An artifact of the assembly scheme is the inclusion of an inoperative chloramphenicol resistance marker, which was part of the original DNA template. This build strategy introduced a new cloning site enabling downstream customization. An example application is the insertion of redox genes PntAB and NADK under an arabinose-inducible promoter, in this case, enabling the immediate and titratable conversion of NADH into NADPH. The nucleotide sequences of “Original pQE80L-SH Template” and “Modified pQE80L-SH Template” depicted are SEQ ID NO:14, and SEQ ID NO:15, respectively.

FIG. 10: Construction of the 16-member hydrogenase plasmid library. The hydrogenase library was developed using a third plasmid donated by donated by Sargent and Lamont (2017). pUNI-HypA2-X is identical to pSU-HypA2-X but uses ampicillin selection and ColE1 replication. Unique restriction enzymes SalI, AatII, and EcoRI flanking the selection marker and origin of replication were attractive features for cloning. Templates for alternative antibiotic markers and origins of replication are widely available through the Inventory of Composable Elements (ICE). In this example, the original pUNI-HypA2-X was linearized by selectively removing the high-copy ColE1 origin. The scarless insertion of low-copy SC101 creates a derivative vector named pUNI-HypA2-X (Amp|SC101). This derivative vector can then be used for a second round of cloning, in this case, replacing the ampicillin selection marker with spectinomycin, yielding a second derivative vector named pUNI-HypA2-X (Spec|SC101). The creation of derivative hydrogenase plasmids is now possible, using a cloning strategy that is almost identical to that used to create HYD (Kan|P15A). First, the derivative pUNI vectors are linearized with EcoRI. Second, HYD (Kan|P15A) is used as a template to amplify the T5-inducible operon and new cloning site. The fusion of these two components (either by ligation or Gibson-style assembly methods) yields the derivative hydrogenase, in the example shown, HYD (Spec|SC101).

FIG. 11: Illustrative guide on testing function and effects of hydrogenase.

FIG. 12: Basic function test of the hydrogenase library. Each of the 16 plasmids of the library were individually transformed into BW25113 E. coli and grown in LB media. In triplicate, cultures were treated with 20 ml of H₂, and incubated overnight 30° C.

FIG. 13: Advanced function test of the hydrogenase library. Each of the 16 plasmids within the library were individually transformed into BW25113 E. coli and grown in EZ Rich containing 55.5 mM acetate. Evidence of H₂ consumption with concomitant drop in CO₂ production and rise in acetate retention were collectively considered evidence of functional activity.

FIG. 14: The E. coli NAD kinase (under control of arabinose promoter) was inserted into HYD|Kan:P15A via the NotI/KpnI cloning site. This modified hydrogenase plasmid was then inserted into BW25113 E. coli. The effects of over-expressing E. coli NAD kinase by applying varying intensities of arabinose induction are explored concomitant with H₂ feeding. (TOP ROW): Biomass formation and H₂ consumption after 24 h incubation. (MIDDLE ROW) Effects of arabinose induction upon total nicotinamide (addition of NAD⁺ and NADH), NADH exclusively, and the NAD⁺/NADH ratio. E. coli transformed with HYD|Kan:P15A but without over-expressed NAD kinase was used as a reference standard. (BOTTOM ROW): Effects of arabinose induction upon NADPH. Performed in triplicate.

FIG. 15: Changes in intracellular NADH and NADPH stimulated by H₂ when BW25113 E. coli is cultivated in LB media (Left panel) NADH (Right panel) (NAPDH) when E. coli is expressing hydrogenase.

FIG. 16: Elevation of intracellular succinate concentration stimulated by H₂ when BW25113 E. coli is cultivated in LB media when E. coli is expressing hydrogenase.

### DETAILED DESCRIPTION OF THE INVENTION

The foregoing aspects and others will be readily appreciated by the skilled artisan from the following description of illustrative embodiments when read in conjunction with the accompanying drawings.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to a value including 10% more than the stated value and 10% less than the stated value.

As used herein, the term “promoter” refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon.

A polynucleotide or amino acid sequence is “heterologous” to an organism or a second polynucleotide or amino acid sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety, or a gene that is not naturally expressed in the target tissue).

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

A homologous variant has a polypeptide sequence that is at least 70%, 75%, 80%, 85%, 90%, 95% or 99% identical to any one of the enzymes described in this specification or in an incorporated reference. The homologous variant comprises or retains amino acid residues that are recognized as conserved for the enzyme. The homologous variant may have non-conserved amino acid residues replaced or found to be of a different amino acid, or amino acid(s) inserted or deleted, but which does not affect or has insignificant effect on the enzymatic activity of the homologous variant. The homologous variant has an enzymatic activity that is identical or essentially identical to the enzymatic activity any one of the enzymes described in this specification or in an incorporated reference. The homologous variant may be found in nature or be an engineered mutant thereof.

The terms “host cell” of “host organism” is used herein to refer to a living biological cell that can be transformed via insertion of an expression vector.

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Particular expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

In some embodiments, a suitable site-specific recombination system comprises use of a recombinase listed in Table 1.

TABLE 1
Recombinases.

| # | Name | Host | Organism | Gene | Accession |
|---|---|---|---|---|---|
| 1 | BSu_xerC | Bacillus subtilis | chromosome | codV | P39776 |
| 2 | BSu_xerD | Bacillus subtilis | chromosome | ripX | P46352 |
| 3 | BSu_ydcL | Bacillus subtilis | chromosome | ydcL | A69774 |
| 4 | CBu_tnpA | Clostridium butyricum | chromosome | tnpA | S40097 |
| 5 | Col1D | Escherichia coli | plasmid F | D | P06615 |
| 6 | CP4-57 | Escherichia coli | chromosome | Int | P32053 |
| 7 | Cre | Escherichia coli | phage P1 | Int | P06956 |
| 8 | D29 | Mycobacterium smegmatis | phage D29 | Int | AAC18476 |
| 9 | DLP12 | Escherichia coli | phage DLP12 | Int | P24218 |
| 10 | DNo_int | Dichelobacter nodosus | chromosome | Orf | AAB00935 |
| 11 | ECo_fimB | Escherichia coli | chromosome | fimB | P04742 |
| 12 | ECo_fimE | Escherichia coli | chromosome | fimE | P04741 |
| 13 | ECo_orf | Escherichia coli | chromosome | b2442 | A65019 |
| 14 | ECo_xerC | Escherichia coli | chromosome | xerC | C37841 |
| 15 | ECo_xerD | Escherichia coli | chromosome | xerD | P21891 |
| 16 | HIn_orf | Haemophilus influenzae | chromosome | orf1572 | P46495 |
| 17 | HIn_rci | Haemophilus influenzae | chromosome | rci | P45198 |
| 18 | HIn_xerC | Haemophilus influenzae | chromosome | xerC | P44818 |
| 19 | HIn_xerD | Haemophilus influenzae | chromosome | xerD | P44630 |
| 20 | HK22 | Escherichia coli | phage HK022 | int | AAF30377 |
| 21 | HP1 | Haemophilus influenzae | phage HP1 | int | P21442 |
| 22 | L2 | Acholeplasma sp. | phage L2 | int | AAA87961 |
| 23 | L5 | Mycobacterium tuberculosis | phage L5 | int | CAA79409 |
| 24 | L54 | Staphylococcus aureus | phage L54 | int | P20709 |
| 25 | Lambda | Escherichia coli | phage lambda | int | AAA96562 |
| 26 | LLe_orf | Lactobacillus leichmannii | chromosome | orf | CAA55635 |
| 27 | LLe_xerC | Lactobacillus leichmannii | chromosome | xerC | CAA59018 |
| 28 | phi10MC | Oenococcus oeni | phage phi10MC | int | AAD00268 |
| 29 | MJa_orf | Methanococcus jannaschi | chromosome | orf | Q57813 |
| 30 | MLe_xerD | Mycobacterium leprae | chromosome | xerD | S72959 |
| 31 | MPa_int | Mycobacterium paratuberculosis | chromosome | int | AAA88834 |
| 32 | MTu_int | Mycobacterium tuberculosis | chromosome | int | B70965 |
| 33 | MTu_xerC | Mycobacterium tuberculosis | chromosome | xerC | Q10815 |
| 34 | MV4 | Lactobacillus delbrueckii | phage MV4 | int | AAC48859 |
| 35 | MX8 | Myxococcus xanthus | phage Mx8 | int | AAC48895 |
| 36 | pAE1 | Alcaligenes eutrophus | plasmid pAE1 | orf | AAA87238 |
| 37 | pCL1 | Chlorobium limicola | plasmid pCL1 | fim | AAB36935 |
| 38 | pDU1 | Nostoc sp. | plasmid pDU1 | orf | AAA17517 |
| 39 | pMEA | Amycolatopsis methanolica | plasmid pMEA300 | orf | AAB00469 |
| 40 | RSp_EF | Rhizobium sp. | plasmid pNG234a | EF | P55429 |
| 41 | RSp_GC | Rhizobium sp. | plasmid pNG234a | GC | P55459 |
| 42 | RSp_QK | Rhizobium sp. | plasmid pNG234a | QK | P55632 |
| 43 | RSp_RA | Rhizobium sp. | plasmid pNG234a | RA | AAB92467 |
| 44 | RSp_RB | Rhizobium sp. | plasmid pNG234a | RB | P55635 |
| 45 | RSp_RC | Rhizobium sp. | plasmid pNG234a | RC | P55636 |
| 46 | RSp_RD | Rhizobium sp. | plasmid pNG234a | RD | P55637 |
| 47 | RSp_RE | Rhizobium sp. | plasmid pNG234a | RE | P55638 |
| 48 | RSp_RF | Rhizobium sp. | plasmid pNG234a | RF | P55639 |
| 49 | pSAM2 | Streptomyces ambofaciens | plasmid pSAM2 | orf | P15435 |
| 50 | pSDL2 | Salmonella dublin | plasmid pSDL2 | resV | A38114 |
| 51 | pSE101 | Saccharopolyspora erythraea | plasmid pSE101 | orf | S41725 |
| 52 | pSE211 | Saccharopolyspora erythraea | plasmid pSE211 | orf | P22877 |
| 53 | pWS58 | Lactobacillus delbrueckii | plasmid pWS58 | orf | CAA90472 |
| 54 | phi-11 | Staphylococcus aureus | phage phill | int | AAA32198 |
| 55 | phi-13 | Staphylococcus aureus | phage phi13 | int | S52761 |
| 56 | phi-80 | Escherichia coli phage | phage phi80 | int | CAA27683 |
| 57 | phi-adh | Lactobacillus gasseri | phage phi-adh | int | JN0535 |
| 58 | phi-CTX | Pseudomonas aeruginosa | phage phiCTX | int | CAA74224 |
| 59 | phi-g1e | Lactobacillus sp. | phage phi-g1e | int | T13182 |
| 60 | phi-LC3 | Lactococcus lactis | phage phiLC3 | int | A47085 |
| 61 | phi-R73 | Escherichia coli | phage phi-R73 | int | A42465 |
| 62 | P186 | Escherichia coli | phage 186 | int | AAC34175 |
| 63 | P2 | Escherichia coli | phage P2 | int | AAD03297 |
| 64 | P21 | Escherichia coli | phage P21 | int | AAC48886 |
| 65 | P22 | Salmonella typhimurium | phage P22 | int | AAF75002 |
| 66 | P4 | Escherichia coli | phage P4 | int | CAA29379 |
| 67 | P434 | Escherichia coli | phage 434 | int | P27078 |
| 68 | PAe_xerC | Pseudomonas aeruginosa | chromosome | sss | AAG08665 |
| 69 | PMi_fimB | Proteus mirabilis | chromosome | fimB | CAB61438 |
| 70 | R721 | Escherichia coli | plasmid IncI2 (R721) | rcb | G45252 |
| 71 | Rci | Escherichia coli | plasmid IncI1 (R64) | rci | P10487 |
| 72 | SF6 | Shigella flexneri | phage Sf6 | int | P37317 |
| 73 | SLP1 | Streptomyces coelicolor | plasmid SLP1 | orf | CAC08268 |
| 74 | IntI3 | Serratia marcescens | chromosome | orf | BAA08929 |
| 75 | SsrA | Methanosarcina acetivorans | plasmid pC2A | ssrA | AAB39744 |
| 76 | SSV1 | Sulfolobus sp. | phage SSV1 | int | CAA30211 |
| 77 | T12 | Streptococcus pyogenes | phage T12 | int | AAC488867 |
| 78 | IntI1 | Escherichia coli | transposon Tn21 | int | AAA82254 |
| 79 | Tn4430 | Bacillus thuringiensis | transposon Tn4430 | int | CAA30491 |
| 80 | Tn5041 | Pseudomonas sp. | transposon Tn5041 | orfI | CAA67462 |
| 81 | Tn5252 | Streptococcus pneumoniae | transposon Tn5252 | int | A55863 |
| 82 | Tn5276 | Lactobacillus lactis | transposon Tn5276 | int | C55205 |
| 83 | Tn554a | Staphylococcus aureus | transposon Tn554 | tnpA | P06696 |
| 84 | Tn554b | Staphylococcus aureus | transposon Tn554 | tnpB | P06697 |
| 85 | IntI2 | Escherichia coli | transposon Tn7 | int | CAA05031 |
| 86 | Tn916 | Entercoccus faecalis | transposon Tn916 | int | P22886 |
| 87 | Tuc | Lactobacillus lactis | phage Tuc2009 | int | AAA32608 |
| 88 | BZo_int | Bergeyella zoohelcum | chromosome | orf | AAA50502 |
| 89 | ASp_xisA | Anabaena sp. | chromosome | xisA | P08862 |
| 90 | ASp_xisC | Anabaena sp. | chromosome | xisC | Q44217 |
| 91 | FLP | Saccharomyces cerevisiae | plasmid 2μ | FLP | J01347 |
| 92 | pKD1 | Kluyveromyces lactis | plasmid pKD1 | FLP | P13783 |
| 93 | pSB2 | Zygosaccharomyces bailii | plasmid pSB2 | FLP | M18274 |
| 94 | pSB3 | Zygosaccharomyces bisporus | plasmid pSB3 | FLP | P13784 |
| 95 | pSM1 | Zygosaccharomyces fermentati | plasmid pSM1 | FLP | P13770 |
| 96 | pSR1 | Zygosaccharomyces rouxii | plasmid pSR1 | FLP | P13785 |
| 97 | HPy_xerC | Helicobacter pylori | chromosome | xerC | C64604 |
| 98 | HPy_xerD | Helicobacter pylori | chromosome | xerD | C64644 |
| 99 | Eco_Rac | Escherichia coli | chromosome | int | P76056 |
| 100 | Eco_Qin | Escherichia coli | chromosome | int | P76168 |
| 101 | CP4-6 | Escherichia coli | chromosome | orf | P71928 |
| 102 | E14 | Escherichia coli | chromosome | int | P75969 |
| 103 | MGo_orf | Mycobacterium gordonae | chromosome | orf | AAB54012 |
| 104 | MLe_xerC | Mycobacterium leprae | chromosome | xerC | CAB10656 |
| 105 | MTu_xerD | Mycobacterium tuberculosis | chromosome | xerD | CAB10958 |
| 106 | pEAF | Escherichia coli | plasmid EAF | rsv | AAC44039 |
| 107 | PFl_xerC | Pseudomonas fluorescens | chromosome | sss | T10461 |
| 108 | PWi_orf | Protothera wickerhamii | mitochondria | ymf42 | T11912 |
| 109 | Sfi21 | Streptococcus thermophilus | phage Sfi21 | int | AAD44095 |
| 110 | phi-r1t | Lactobacillus lactis | phage r1t | int | AAB18676 |
| 111 | STy_xerC | Salmonella typhimurium | chromosome | xerC | P55888 |
| 112 | STy_xerD | Salmonella typhimurium | chromosome | xerD | P55889 |
| 113 | SSp_orf | Synechocystis sp. | chromosome | orf | BAA16682 |
| 114 | DNo_orf | Dichelobacter nodosus | chromosome | orf | AAB00935 |
| 115 | VCh_orf | Vibrio cholerae | chromosome | orf | AAC44230 |
| 116 | MMa_xerC | Methanothermobacter | chromosome | xerC | D69219 |
| | | marburgensis | | | |
| 117 | ECo_orf2 | Escherichia coli | chromosome | intB | P39347 |
| 118 | SIn_orf | Salmonella infantis | chromosome | orf | J03391 |
| 119 | BK-T | Lactococcus lactis | phage BK-T | int | T13262 |
| 120 | phi-42 | Staphylococcus aureus | phage phi42 | int | AAA91615 |
| 121 | FRAT1 | Mycobacterium sp. | phage FRAT1 | int | P25426 |
| 122 | HZe_vlf1 | Helicoverpa zea | chromosome | vlf1 | AAA58702 |
| 123 | pKW1 | Kluveromyces waltii | plasmid pKW1 | FLP | X56553 |
| 124 | CBu_tnpB | Clostridium butyricum | chromosome | tnpB | S40098 |
| 125 | S2 | Haemophilus influenzae | phage S2 | int | CAA96221 |
| 126 | NBU1 | Bacteroides uniformis | plasmid NBU1 | int | AAF74437 |
| 127 | Tn1545 | Streptococcus pneumoniae | transposon Tn1545 | int | P27451 |
| 128 | T270 | Streptococcus pyogenes | phage T270 | int | AAA85500 |
| 129 | PMi_xerC | Proteus mirabilis | chromosome | xerC | AAB87500 |
| 130 | PMi_xerD | Proteus mirabilis | chromosome | xerD | AAB87499 |
| 131 | phiV | Shigella flexneri | phage V | int | AAB72135 |
| 132 | O1205 | Streptococcus thermophilus | phage O1205 | int | T13289 |
| 133 | Tn4556 | Streptomyces fradiae | transposon Tn4556 | int | P20184 |
| 134 | MS6 | Mycobacterium sp. | phage Ms6 | int | AAD03774 |
| 135 | pFAJ | Rhodococcus erythropolis | plasmid pFAJ2600 | pmrA | AAC45806 |
| 136 | SMa_xerC | Serratia marcescens | chromosome | xerC | AAC46276 |
| 137 | pTiA6 | Agrobacterium tumefaciens | plasmid pTiA6NC | int | AAB91569 |
| 138 | AAe_orf | Aquifex aeolicus | chromosome | int | G70397 |
| 139 | Tn557 | Staphylococcus aureus | transposon Tn557 | int | AAC28969 |
| 140 | EAe_int | Enterobacter aerogenes | chromosome | int | AAB95339 |
| 141 | SF2 | Shigella flexneri | phage Sf2 | int | AAC39270 |
| 142 | ECo_yfdB | Escherichia coli | chromosome | yfdB | P37326 |
| 143 | RP3 | Streptomyces rimosus | phage RP3 | int | X80661 |
| 144 | VWB | Streptomyces venezuelae | phage VWB | int | CAA03882 |
| 145 | SEx_vlf1 | Spodoptera exigua | chromosome | vlf1 | AAF33611 |
| 146 | STy_rci | Salmonella typhimurium | chromosome | rci | AAC38070 |
| 147 | PPu_orf | Pseudomonas putida | chromosome | orf | CAA06238 |
| 148 | A2 | Lactobacillus casei | phage A2 | int | CAA73344 |
| 149 | pECE1 | Aquifex aeolicus | plasmid ece1 | int | AAC07955 |
| 150 | MLo_int | Mesorhizobium loti | chromosome | intS | AAC24508 |
| 151 | SRu_orf | Selenomonas ruminantium | chromosome | orf | BAA24921 |
| 152 | pQPRS | Coxiella burnetti | plasmid pQPRS | int | CAA75853 |
| 153 | PRe_orf | Panagrellus redivivus | chromosome | orf | CAA43185 |
| 154 | CEl_orf | Caenorhabditis elegans | chromosome | orf | Z82079 |
| 155 | IntI4 | Vibrio cholerae | chromosome | intI4 | AAF71178 |
| 156 | SMu_orf | Streptococcus mutans NG8 | chromosome | orfA | AAC17173 |
| 157 | phiU | Rhizobium leguminosarum | phage phiU | int | BAA25885 |
| 158 | PHo_xerC | Pyrococcus horikoshii | chromosome | xerC | B71194 |
| 159 | RCa_orf1 | Rhodobacter capsulatus | chromosome | orf1 | T03499 |
| 160 | RCa_orf2 | Rhodobacter capsulatus | chromosome | orf2 | T03567 |
| 161 | Tn5382 | Enterococcus faecium | transposon Tn5382 | int | AAC34799 |
| 162 | psiM2 | Methanothermobacter | phage PsiM2 | int | T12745 |
| | | marburgensis | | | |
| 163 | STy_orf | Salmonella typhimurium | chromosome | orf | T03001 |
| 164 | MTu_orf | Mycobacterium tuberculosis | chromosome | Rv2659c | G70966 |
| 165 | TPa_xerC | Treponema pallidum | chromosome | codV | AAC65375 |
| 166 | TPa_xerD | Treponema pallidum | chromosome | xprB | AAC65379 |
| 167 | CTr_xerC | Chlamydia trachomatis | chromosome | xerC | AAC67942 |
| 168 | CTr_xerD | Chlamydia trachomatis | chromosome | xerD | AAC68462 |
| 169 | phiPVL | Staphylococcus aureus | phage phiPVL | int | BAA31902 |
| 170 | pNL1 | Sphingomonas aromaticivorans | plasmid pNL1 | int | AAD03886 |
| 171 | CP4-157 | Escherichia coli O157:H7 | chromosome | int | AAC31482 |
| 172 | SAu_xerD | Staphylococcus aureus | chromosome | xerD | AAC64162 |
| 173 | YPe_orf | Yersinia pestis | chromosome | orf | AAC69581 |
| 174 | RPr_xerD | Rickettsia prowazekii | chromosome | xerD | B71693 |
| 175 | RPr_xerC | Rickettsia prowazekii | chromosome | xerC | B71643 |
| 176 | VCh_SXT | Vibrio cholerae | chromosome | orf | AAF93686 |
| 177 | AAc_orf | Actinob. actinomycetemcomitans | chromosome | orf | AAC70901 |
| 178 | MAV1 | Mycoplasma arthritidis | chromosome | int | AAC33780 |
| 179 | fOg44 | Oenococcus oeni | phage fOg44 | int | AAD10711 |
| 180 | SFX | Shigella flexneri | phage SFX | int | AAD10295 |
| 181 | Tn4371 | Ralstonia eutropha | transposon Tn4371 | int | CAA71790 |
| 182 | HPy_orf | Helicobacter pylori | chromosome | orf | A71869 |
| 183 | CPn_xerC | Chlamydia pneumoniae | chromosome | xerD | BAA99231 |
| 184 | CPn_xerD | Chlamydia pneumoniae | chromosome | xerC | BAA98236 |
| 185 | K139 | Vibrio cholerae | phage K139 | int | AAD22068 |
| 186 | PPu_orf2 | Pseudomonas putida | chromosome | orf | BAA75916 |
| 187 | pPZG | Pantoea citrea | plasmid pPZG500 | int | AAD21210 |
| 188 | H19J | Escherichia coli | phage H19J | int | CAB38715 |
| 189 | phi304L | Corynebacterium glutamicum | phage phi304L | int | CAB38562 |
| 190 | SCo_orf | Streptomyces coelicolor | chromosome | orf | T36198 |
| 191 | phi_16 | Corynebacterium glutamicum | phage phi16 | int | CAA73074 |
| 192 | BHa_xerC | Bacillus halodurans | chromosome | codV | BAB06184 |
| 193 | XFa_xerC | Xylella fastidiosa | chromosome | xerC | AAF84292 |
| 194 | BHa_xerD | Bacillus halodurans | chromosome | xerD | BAB05248 |
| 195 | PAe_xerD | Pseudomonas aeruginosa | chromosome | xerD | AAG07125 |
| 196 | VCh_xerC | Vibrio cholerae | chromosome | xerC | AAF93305 |
| 197 | VCh_xerD | Vibrio cholerae | chromosome | xerD | AAF95562 |
| 198 | NMa_xerC | Neisseria meningitidis ser. A | chromosome | xerC | CAB83879 |
| 199 | NMb_xerC | Neisseria meningitidis ser. B | chromosome | xerC | AAF42202 |
| 200 | XFa_xerD | Xylella fastidiosa | chromosome | xerD | AAF84234 |
| 201 | CMu_xerC | Chlamydia muridarum | chromosome | xerC | AAF73578 |
| 202 | SAu_xerC | Staphylococcus aureus | chromosome | xerC | AAF89877 |
| 203 | NMa_xerD | Neisseria meningitidis ser. B | chromosome | xerD | AAF41164 |
| 204 | NMb_xerD | Neisseria meningitidis ser. A | chromosome | xerD | CAB84234 |
| 205 | CMu_xerD | Chlamydia muridarum | chromosome | xerD | AAF39124 |
| 206 | PAb_xerD | Pyrococcus abysii | chromosome | xerD | A75153 |
| 207 | pI3 | Deinococcus radiodurans | plasmid pI3 | ResU | AAF44051 |
| 208 | pTiSAK | Agrobacterium tumefaciens | plasmid TiSAKURA | orf36 | BAA87661 |
| 209 | HPj_xerC | Helicobacter pylori J | chromosome | xerC | B71910 |
| 210 | TMa_xerC | Thermotoga maritima | chromosome | xerC | D72312 |
| 211 | CJe_xerD | Campylobacter jejuni | chromosome | xerD | CAB73128 |
| 212 | APe_xerD | Aeropyrum pernix | chromosome | xerD | G72672 |
| 213 | PSy_orf | Pseudomonas syringae | chromosome | orfF | CAB96970 |
| 214 | MM1 | Streptococcus pneumoniae | phage MM1 | int | CAB96616 |
| 215 | XNi_vlf1 | Xestia nigrum | chromosome | vlf1 | AAF05239 |
| 216 | PXy_vlf1 | Plutella xylostella | chromosome | vlf1 | AAG27387 |
| 217 | pXO1-132 | Bacillus anthracis | plasmid pXO1 | 132 | D59107 |
| 218 | Tn4555 | Bacteroides fragilis | transposon Tn4555 | int | AAB53787 |
| 219 | DRa_xer | Deinococcus radiodurans | chromosome | xerD | G75636 |
| 220 | BJa_int | Bradyrhizobium japonicum | chromosome | intA | AAF64651 |
| 221 | BHa_orf4 | Bacillus halodurans | chromosome | BH2349 | BAB06068 |
| 222 | pXO1-103 | Bacillus anthracis | plasmid pXO1 | 103 | G59103 |
| 223 | PAe_orf2 | Pseudomonas aeruginosa | chromosome | orf2 | AAG04117 |
| 224 | pLGV440 | Chlamydia trachomatis | plasmid pLGV440 | orf8 | P08788 |
| 225 | Tn5520 | Bacteroides fragilis | transposon Tn5520 | bipH | AAC80279 |
| 226 | pNL1_tnpA | Sphingomonas aromaticivorans | plasmid pNL1 | tnpA | AAD03922 |
| 227 | CTr_orf | Chlamydia trachomatis | chromosome | orf1 | S44160 |
| 228 | BHa_orf1 | Bacillus halodurans | chromosome | BH3551 | BAB07270 |
| 229 | phi-933W | Escherichia coli | phage 933W | int | AAD25406 |
| 230 | CPs_orf1 | Chlamydia psittaci | chromosome | orf | B39999 |
| 231 | VCh_orf2 | Vibrio cholerae | chromosome | VC1758 | AAF94908 |
| 232 | DRa_orf2 | Deinococcus radiodurans | chromosome | orf2 | F75611 |
| 233 | pCPnE1 | Chlamydophila pneumoniae | plasmid pCPnE1 | orf2 | CAA57585 |
| 234 | ECo_intB | Escherichia coli | chromosome | intB | AAD37509 |
| 235 | UUr_xerC | Ureaplasma urealyticum | chromosome | xerC | AAF30630 |
| 236 | HK97 | Escherichia coli | phage HK97 | int | AAF31094 |
| 237 | TPW22 | Lactococcus sp. | phage TPW22 | int | AAF12706 |
| 238 | APSE-1 | Acyrthosiphon pisum | phage APSE-1 | int | AAF03981 |
| 239 | pURB500 | Methanococcus maripaludis | plasmid pURB500 | int | AAC45247 |
| 240 | SFl_int | Shigella flexneri | chromosome | int | AAD44730 |
| 241 | UUr_xerD | Ureaplasma urealyticum | chromosome | ripX | AAF30551 |
| 242 | Wphi | Escherichia coli | phage Wphi | int | CAB54522 |
| 243 | BHa_orf2 | Bacillus halodurans | chromosome | BH2364 | BAB06083 |
| 244 | SEn_int | Salmonella enterica | chromosome | intI5 | AAG03003 |
| 245 | pCP1 | Deinococcus radiodurans | plasmid pCP1 | xerD | AAF12667 |
| 246 | SCo_int | Streptomyces coelicolor | chromosome | int | CAB71253 |
| 247 | PRi1724 | Agrobacterium rhizogenes | plasmid pRi1724 | orf9 | BAB16128 |
| 248 | SCo_traS | Streptomyces coelicolor | chromosome | traS | T35465 |
| 249 | HPy_orf1 | Helicobacter pylori | chromosome | orf | A71870 |
| 250 | XFa_orf1 | Xylella fastidiosa | chromosome | XF2530 | AAF85328 |
| 251 | UUr_codV | Ureaplasma urealyticum | chromosome | codV | AAF30942 |
| 252 | pXO1-18 | Bacillus anthracis | plasmid pXO1 | 18 | B59093 |
| 253 | CPs_orf2 | Chlamydia psittaci | chromosome | orf2 | A39999 |
| 254 | SPBc2 | Bacillus subtilis | phage SPBc2 | yopP | T12850 |
| 255 | D3 | Pseudomonas aeruginosa | phage D3 | int | AAF04808 |
| 256 | XFa_orf2 | Xylella fastidiosa | chromosome | XF1642 | AAF84451 |
| 257 | XFa_orf3 | Xylella fastidiosa | chromosome | XF0678 | AAF83488 |
| 258 | pLGV440-2 | Chlamydia trachomatis | plasmid pLGV440 | N1 | S01180 |
| 259 | pB171 | Escherichia coli | plasmid pB171 | rsvB | BAA84906 |
| 260 | DRa_orf3 | Deinococcus radiodurans | chromosome | orf | C75509 |
| 261 | CPZ-55 | Escherichia coli | phage CPZ-55 | int | P76542 |
| 262 | ICESt1 | Streptococcus thermophilus | transposon ICESt1 | int | CAB70622 |
| 263 | pGP7-D | Chlamydia trachomatis | plasmid pGP7-D | TCA01 | AAF39715 |
| 264 | XFa_orf4 | Xylella fastidiosa | chromosome | XF1718 | AAF84527 |
| 265 | HIn_orf2 | Haemophilus influenzae | chromosome | int | AAF27347 |
| 266 | DNo_orf2 | Dichelobacter nodosus | chromosome | intC | CAB57348 |
| 267 | NBU2 | Bacteroides fragilis | transposon NBU2 | intN2 | AAF74726 |
| 268 | pCol1B | Shigella sonnei | plasmid Col1B-P9 | resA | BAA75108 |
| 269 | PSy_orf4 | Pseudomonas syringiae | chromosome | orf | CAC14205 |
| 270 | Tn4652 | Pseudomonas putida | transposon Tn4652 | orf5 | AAD44277 |
| 271 | pLGV440-3 | Chlamydia trachomatis | plasmid pLGV440 | orf7 | P10561 |
| 272 | pF | Escherichia coli | plasmid F | int | BAA97902 |
| 273 | BHa_orf3 | Bacillus halodurans | chromosome | BH4039 | BAB07758 |
| 274 | XFa_orf5 | Xylella fastidiosa | chromosome | XF2132 | AAF84931 |
| 275 | pNRC100_1 | Halobacterium sp. | plasmid pNRC100 | H0618 | T08273 |
| 276 | SDy_orf | Shigella dysenteriae | chromosome | int | AAF28112 |
| 277 | pQpRS_2 | Coxiella burnetti | plasmid pQpRS | orf410 | CAA75839 |
| 278 | PMu_rci | Pasteurella multocida | chromosome | rci | AAF68420 |
| 279 | SPBc2 | Bacillus subtilis | phage SPBc2 | yomM | AAC13009 |
| 280 | PPa_int | Pseudomonas pavonaceae | chromosome | intP | CAB65361 |
| 281 | pKLC102 | Pseudomonas aeruginosa | plasmid pKLC102 | xerC | AAG02084 |
| 282 | XFa_orf6 | Xylella fastidiosa | chromosome | XF0631 | AAF83441 |
| 283 | SCo_orf3 | Streptomyces coelicolor | chromosome | int | CAC14368 |
| 284 | LLa_orf | Lactococcus lactis | chromosome | orf3 | AAF86683 |
| 285 | MSp_orf | Mycobacterium sp. | chromosome | intM | CAB65286 |
| 286 | pNL1_tnpB | Sphingomonas aromaticivorans | plasmid pNL1 | tnpB | AAD03921 |
| 287 | XFa_orf7 | Xylella fastidiosa | chromosome | XF0968 | AAF83778 |
| 288 | ECo_orf5 | Escherichia coli | chromosome | int | AAF06962 |
| 289 | AGe_vlf1 | Anticarsia gemmatalis | chromosome | vlf-1 | AAD54607 |
| 290 | pLH1 | Lactobacillus helveticus | plasmid pLH1 | orf195 | CAA10964 |
| 291 | SAu_orf2 | Staphylococcus aureus | chromosome | orf | AAG29618 |
| 292 | LDi_vlf1 | Lymantria dispar | chromosome | vlf-1 | AAC70272 |
| 293 | OPs_vlf1 | Orgyia pseudotsugata | chromosome | vlf-1 | AAC59079 |
| 294 | SCo_orf2 | Streptomyces coelicolor | chromosome | int | CAC08306 |
| 295 | BBu_orf | Borrelia burgdorferi | chromosome | orf6 | AAC34963 |
| 296 | pNOB8 | Sulfolobus sp. | plasmid pNOB8 | orf101 | T31031 |
| 297 | pMT1 | Yersinia pestis | plasmid pMT1 | T1101 | T15016 |
| 298 | ACa_vlf1 | Autographica californica | chromosome | vlf-1 | AAA66707 |
| 299 | VCh_orf3 | Vibrio cholerae | chromosome | VC0821 | AAF96190 |
| 300 | BMo_vlf1 | Bombyx mori | chromosome | vlf-1 | AAC63749 |
| 301 | phi-PV83 | Staphylococcus aureus | phage PV83 | int | BAA97808 |
| 302 | PGi_orf | Porphyromonas gingivalis | chromosome | orf6 | BAA35089 |
| 303 | AFu_orf | Archaeoglobus fulgidus | chromosome | AF0082 | B69260 |
| 304 | pCHL1 | Chlamydia trachomatis | plasmid pCHL1 | orf7 | AAA91567 |
| 305 | pR27 | Salmonella typhi | plasmid R27 | orf | AAF70020 |
| 306 | APe_orf | Aeropyrum pernix | chromosome | APE0818 | E72674 |
| 307 | PSy_orf2 | Pseudomonas syringiae | chromosome | orfA | CAB96965 |
| 308 | pNRC100_2 | Halobacterium sp. | plasmid pNRC100 | H0928 | T08297 |
| 309 | MJa_orf2 | Methanococcus jannaschi | chromosome | MJ0770 | Q58180 |
| 310 | phi16-3 | Rhizobium sp. | phage 16-3 | int | CAB54831 |
| 311 | pCP32-1 | Borrelia burgdorferi | plasmid cp32-1 | BBP37 | AAF07426 |
| 312 | SAl_orf | Streptomyces albus | chromosome | orf | AAD46512 |
| 313 | pNRC100_3 | Halobacterium sp. | plasmid pNRC100 | H1373 | T08333 |
| 314 | VCh_orf4 | Vibrio cholerae | chromosome | VC0185 | AAF93361 |
| 315 | Tec2 | Euplotes crassus | transposon Tec2 | orf2B | AAA91341 |
| 316 | Tec1 | Euplotes crassus | transposon Tec1 | orf2B | AAA91341 |
| 317 | PPu_orf3 | Pseudomonas putida | chromosome | orf101 | CAB54061 |
| 318 | pCP32 | Borrelia hermsii | plasmid cp32 | orf6 | AAF28881 |
| 319 | NMe_int | Neisseria meningitidis | chromosome | int | CAB84481 |
| 320 | pCP32-4 | Borrelia burgdorferi | plasmid cp32-4 | BBR38 | AAF07512 |
| 321 | pCP18 | Borrelia burgdorferi | plasmid cp18 | orf6 | AAB63432 |
| 322 | pCP18-2 | Borrelia burgdorferi | plasmid cp 18-2 | orf27 | AAF29799 |
| 323 | Tn5401 | Bacillus thuringensis | transposon Tn5401 | int | P27451 |
| 324 | SMi_xerD | Streptococcus mitis | chromosome | xerD | CAC19443 |
| 325 | SPn_xerD | Streptococcus pneumoniae | chromosome | xerD | CAC19448 |
| 326 | EFa_orf | Enterococcus faecium | chromosome | intD | AAG42074 |
| 327 | VT1 | Escherichia coli O157:H7 | phage VT1-Sakai | int | BAB19626 |
| 328 | psiM100 | Methanothermobacter wolfeii | phage psiM100 | int | AAG39942 |
| 329 | CP-933C | Escherichia coli O157:H7 | phage 933C | Z1835 | AAG55933 |
| 330 | CP-933I | Escherichia coli O157:H7 | phage 933I | Z0324 | AAG54584 |
| 331 | CP-933M | Escherichia coli O157:H7 | phage 933M | Z1323 | AAG55457 |
| 332 | CP-933U | Escherichia coli O157:H7 | phage 933U | intU | AAG57039 |
| 333 | CP-933T | Escherichia coli O157:H7 | phage 933T | intT | AAG56898 |
| 334 | CP-933N | Escherichia coli O157:H7 | phage 933N | intN | AAG55869 |
| 335 | CP-933O | Escherichia coli O157:H7 | phage 933O | intO | AAG56112 |
| 336 | bIL310 | Lactococcus lactis | phage bIL310 | orf1 | AAK08405 |
| 337 | bIL311 | Lactococcus lactis | phage bIL311 | int | AAK08433 |
| 338 | SPy_orf5 | Streptococcus pyogenes | chromosome | int4 | AAK34767 |
| 339 | bIL309 | Lactococcus lactis | phage bIL309 | int | AAK08349 |
| 340 | bIL312 | Lactococcus lactis | phage biL312 | int | AAK08454 |
| 341 | SPy_orf2 | Streptococcus pyogenes | chromosome | int3 | AAK33851 |
| 342 | SPy_orf4 | Streptococcus pyogenes | chromosome | int2 | AAK34288 |
| 343 | bIL286 | Lactococcus lactis | phage bIL286 | int | AAK08288 |
| 344 | LLa_xerD | Lactococcus lactis | chromosome | xerD | AAK04743 |
| 345 | LLa_ymfD | Lactococcus lactis | chromosome | ymfD | AAK05330 |
| 346 | SPy_orf3 | Streptococcus pyogenes | chromosome | spy1196 | AAK34058 |
| 347 | SPy_orf1 | Streptococcus pyogenes | chromosome | spy0365 | AAK33410 |
| 348 | LLa_orf2 | Lactococcus lactis | chromosome | ynbA | AAK05376 |
| 349 | ECo_orf7 | Escherichia coli O157:H7 | chromosome | Z4313 | AAG58098 |
| 350 | ECo_orf6 | Escherichia coli O157:H7 | chromosome | Z1120 | AAG55265 |
| 351 | pMLa | Mesorhizobium loti | plasmid pMLa | mll9356 | BAB54967 |
| 352 | pMLb | Mesorhizobium loti | plasmid pMLb | mlr9649 | BAB54839 |
| 353 | pRi_orf2 | Rhizobium rhizogenes | plasmid pRi | ri136 | BAB16255 |
| 354 | MLo_orf1 | Mezorhizobium loti | chromosome | mll8495 | BAB54366 |
| 355 | MLo_orf2 | Mezorhizobium loti | chromosome | mll7973 | BAB53631 |
| 356 | MLo_orf3 | Mezorhizobium loti | chromosome | mlr7741 | BAB54140 |
| 357 | MLo_orf4 | Mezorhizobium loti | chromosome | mlr6952 | BAB53138 |
| 358 | SEn_orf2 | Salmonella enterica | chromosome | int2 | AF261825 |
| 359 | MLo_orf5 | Mezorhizobium loti | chromosome | mll5763 | BAB52151 |
| 360 | ECo_orf8 | Escherichia coli | chromosome | ILG1 | AAK49816 |
| 361 | MLo_orf6 | Mezorhizobium loti | chromosome | mlr0958 | BAB48432 |
| 362 | CCr_orf1 | Caulobacter crescentus | chromosome | CC2681 | AAK24647 |
| 363 | MLo_orf7 | Mezorhizobium loti | chromosome | mll4043 | BAB50796 |
| 364 | MLo_orf8 | Mezorhizobium loti | chromosome | mll0487 | BAB48065 |
| 365 | MLo_orf9 | Mezorhizobium loti | chromosome | mlr0475 | BAB48054 |
| 366 | phi-ETA | Staphylococcus aureus | phage phi-ETA | orf1 | BAA97587 |
| 367 | CCr_xerD | Caulobacter crescentus | chromosome | CC3006 | AAK24968 |
| 368 | CCr_xerC | Caulobacter crescentus | chromosome | CC0344 | AAK22331 |
| 369 | pRVS1 | Vibrio salmonicida | plasmid pRVS1 | int | CAC35342 |
| 370 | phiSLT | Staphylococcus aureus | phage phi-SLT | int | BAB21695 |
| 371 | SSo_xer | Sulfolobus solfataricus | chromosome | xerCD | AAK40704 |
| 372 | CW459 | Clostridium perfringens | transposon CW459 | int459 | AAK17958 |
| 373 | MPu_xerC | Mycoplasma pulmonis | chromosome | MY5310 | CAC13704 |
| 374 | TVo_xerC | Thermoplasma volcanium | chromosome | xerC | BAB59407 |
| 375 | TAc_xerC | Thermoplasma acidophilum | chromosome | Ta1314 | CAC12435 |
| 376 | TVo_orf1 | Thermoplasma volcanium | chromosome | orf1 | BAB59869 |
| 377 | SEn_orf2 | Salmonella enterica | chromosome | S020 | AAK02039 |
| 378 | PMu_xerC | Pasteurella multocida | chromosome | xerC | AAK03785 |
| 379 | PMu_xerD | Pasteurella multocida | chromosome | xerD | AAK02177 |
| 380 | MLo_xerD | Mesorhizobium loti | chromosome | mlr3575 | NP_104652 |
| 381 | DRa_orf4 | Deinococcus radiodurans | chromosome | xerD | AAF12544 |
| 382 | HSp_orf1 | Halobacterium sp. | chromosome | ssrA | AAG19292 |
| 383 | PMu_orf1 | Pasteurella multocida | chromosome | slpA | AAK03853 |
| 384 | PGi_xerC | Porphyromonas gingivalis | chromosome | PG1732 | |
| 385 | PGi_xerD | Porphyromonas gingivalis | chromosome | PG0386 | |
| 386 | RCa_orf3 | Rhodobacter capsulatus | chromosome | orf | U57682 |
| 387 | MLo_orf10 | Mesorhizobium loti | chromosome | mlr9321 | NP_085850 |
| 388 | MLo_orf11 | Mesorhizobium loti | chromosome | mlr9323 | NP_085851 |
| 389 | MLo_orf12 | Mesorhizobium loti | chromosome | mlr9324 | NP_085852 |
| 390 | MLo_orf13 | Mesorhizobium loti | chromosome | mll9328 | NP_085856 |
| 391 | MLo_orf14 | Mesorhizobium loti | chromosome | mll9329 | NP_085857 |
| 392 | MLo_orf15 | Mesorhizobium loti | chromosome | mll9330 | NP_085858 |
| 393 | MLo_orf16 | Mesorhizobium loti | chromosome | mll9331 | NP_085859 |

In some embodiments, the suitable recombinases are the recombinases listed as numbers 7, 12, 93, 95, 97, and 98 in Table 1.

### Host Cells

In some embodiments, the host cells are genetically modified in that heterologous nucleic acid have been introduced into the host cells, and as such the genetically modified host cells do not occur in nature. The suitable host cell is one capable of expressing a nucleic acid construct encoding one or more enzymes described herein. The gene(s) encoding the enzyme(s) may be heterologous to the host cell or the gene may be native to the host cell but is operatively linked to a heterologous promoter and one or more control regions which result in a higher expression of the gene in the host cell.

Each introduced enzyme can be native or heterologous to the host cell. Where the enzyme is native to the host cell, the host cell is genetically modified to modulate expression of the enzyme. This modification can involve the modification of the chromosomal gene encoding the enzyme in the host cell or a nucleic acid construct encoding the gene of the enzyme is introduced into the host cell. One of the effects of the modification is the expression of the enzyme is modulated in the host cell, such as the increased expression of the enzyme in the host cell as compared to the expression of the enzyme in an unmodified host cell.

The genetically modified host cell can be any bacterial cell capable of production of the compound of interest in accordance with the methods of the invention. The present invention can be used comprising any biosynthetic pathway to produce any compound/molecule of interest.

In some embodiments, the host cell is a prokaryotic cell, such as a bacterial cell. In some embodiments, the host cell is a bacterial cell selected from the Escherichia, Enterobacter, Azotobacter, Erwinia, Bacillus, Pseudomonas, Klebsielia, Proteus, Salmonella, Serratia, Shigella, Ralstonia, Rhizobia, or Vitreoscilla taxonomical class. Bacterial host cells suitable for the invention include, but are not limited to, Escherichia, Corynebacterium, Pseudomonas, Streptomyces, and Bacillus. In some embodiments, the Escherichia cell is an E. coli, E. albertii, E. fergusonii, E. hermanii, E. marmotae, or E. vulneris. In some embodiments, the Corynebacterium cell is Corynebacterium glutamicum, Corynebacterium kroppenstedtii, Corynebacterium alimapuense, Corynebacterium amycolatum, Corynebacterium diphtheriae, Corynebacterium efficiens, Corynebacterium jeikeium, Corynebacterium macginleyi, Corynebacterium matruchotii, Corynebacterium minutissimum, Corynebacterium renale, Corynebacterium striatum, Corynebacterium ulcerans, Corynebacterium urealyticum, or Corynebacterium uropygiale. In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, or P. plecoglossicida. In some embodiments, the Streptomyces cell is a S. coelicolor, S. lividans, S. venezuelae, S. ambofaciens, S. avermitilis, S. albus, or S. scabies. In some embodiments, the Bacillus cell is a B. subtilis, B. megaterium, B. licheniformis, B. anthracis, B. amyloliquefaciens, B. pumilus, B. brevis, B. aminovorans, or B. fusiformis. In some embodiments the bacterial cell is a Gram-positive bacterium, such as a Streptomyces species, such as any Streptomyces species or strain taught herein.

The genetically modified host cell can be any yeast capable of production of the compound in accordance with the methods of the invention.

In some embodiments, the host cell is a yeast. Yeast host cells suitable for the invention include, but are not limited to, Yarrowia, Candida, Bebaromyces, Saccharomyces, Schizosaccharomyces and Pichia cells. In one embodiment, Saccharomyces cerevisae is the host cell. In one embodiment, the yeast host cell is a species of Candida, including but not limited to C. tropicalis, C. maltosa, C. apicola, C. paratropicalis, C. albicans, C. cloacae, C. guillermondii, C. intermedia, C. lipolytica, C. panapsilosis and C. zeylenoides. In one embodiment, Candida tropicalis is the host cell.

In some embodiments, the yeast host cell is a non-oleaginous yeast. In some embodiments, the yeast host cell is a basidiomycete. In some embodiments, the yeast host cell is an oleaginous yeast. In some embodiments, the oleaginous yeast is a Rhodosporidium species. In some embodiments, the Rhodosporidium species is Rhodosporidium toruloides. In some embodiments, the Rhodosporidium toruloides is strain IFO 0880.

In some embodiments, the present invention provides for one or more of the following: A robust and sustainable bioeconomy can only be realized through the industrial-scale, carbon-neutral synthesis of fuels, chemicals, and materials. Biofuels, along with a growing number of other sustainable products, are made almost exclusively via fermentation, the age-old technology used to produce foods such as wine, beer, and cheese. Current commercial methods to produce ethanol biofuel from sugar or starches waste more than 30% of the carbon in the feedstock as carbon dioxide (CO₂) in the fermentation step alone. This waste limits product yields and squanders valuable feedstock carbon as greenhouse gas CO₂. Preventing the loss of carbon as CO₂ during bioconversion, or directly incorporating external CO₂ as a feedstock into bioconversions, would revolutionize bioprocessing by increasing the product yield per unit of carbon input by more than 50%.

The application of biology to sustainable uses of waste carbon resources for the generation of energy, intermediates, and final products—i.e., supplanting the “bioeconomy”—provides economic, environmental, social, and national security benefits and offers a promising means of carbon management.

In some embodiments, the present invention may comprise one or more of the following aspects.

Aspect 1. Hydrogenase activity requires heterotetramer HoxFUYX and nine maturation proteins. Using parts kindly provided by Lamont and Sargent (2017), all 13 genes are consolidated into a single plasmid, which is governed by medium copy origin of replication P15A and kanamycin selection marker. The plasmid is divided in two operon such that the catalytic heterotetramer (hoxFUYHWI) is controlled by the IPTG-inducible T5 promoter and the support genes (hypA2B2F2CDEX) are controlled by the constitutive tatA promoter.

Aspect 2. Within the consolidated hydrogenase, we also inserted a new cloning site composed of NotI and KpnI restriction enzyme sites, enabling downstream customization of hydrogenase. For example, we inserted a third operon encoding E. coli PntAB, and/or NADK, under control of arabinose-inducible promoter. Whereas the hydrogenase alone is responsible for regenerating NADH from H₂, this three-operon plasmid can instead regenerate NADPH via the following two-step reaction:

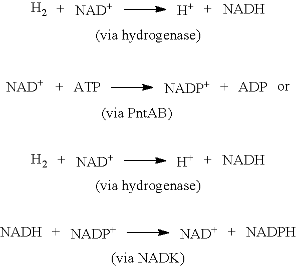

Many other genes can also be inserted within the hydrogenase. For example, we have also inserted the gene encoding the type III PKS RppA responsible for red pigment flaviolin biosynthesis, under control of IPTG-inducible T7 promoter. This cloning site is advantageous for the end-user because it eliminates the requirement of maintaining a second antibiotic selection marker, decreases the metabolic burden placed upon the host, and normalizes statistical data by coupling hydrogenase plasmid replication to target pathway replication.

Aspect 3. Using the gene components provided by Lamont and Sargent (2017), we invented a modular cloning approach that enabled the construction of a library of 16 hydrogenases. The library contains every permutation of these four antibiotic selection markers: Amp, Kan, Chlor, Spec; The library also contains every permutation of these four origins of replication: ColE1, P15A, SC101, and BBR1. All 16 hydrogenases possess the KpnI-NotI cloning site because this cloning site is inherited as part of the modular assembly process. A theoretically infinite number of variations could be created through the modular process. The process can be performed either ourselves or by an external end-user, guided by instructions provided herein. The variability of selection markers and origins of replication allows an end-user to complement the appropriate hydrogenase with their own biosynthetic platform without the need to subclone either component into a new backbone. Additionally, our own data shows that hydrogenase activity is affected by the origin of replication, meaning that it is also possible to tailor hydrogenase activity by choice of origin of replication.

Aspect 4. The 13-gene hydrogenase was also chromosomally inserted into BW25113 E. coli under control of the IPTG-inducible T5 promoter. Shotgun proteomics similarly demonstrate that all 13 proteins are expressed. The host is amenable to accepting any other plasmid encoding a biosynthetic gene of interest. Thus, a platform for plasmid-free H₂-supported biosynthesis is provided.

Aspect 5. Shotgun proteomics demonstrate that all 13 proteins are expressed when the plasmid is transformed into BW25113 E. coli. Furthermore, proteomic analysis also demonstrates hydrogenase expression in E. coli, when grown in LB media, is comparable to the native C. necator H16, when grown autotrophically.

Aspect 6. Cell-free assays of hydrogenase expressed from E. coli revealed that activity is comparable to hydrogenase when expressed in original host.

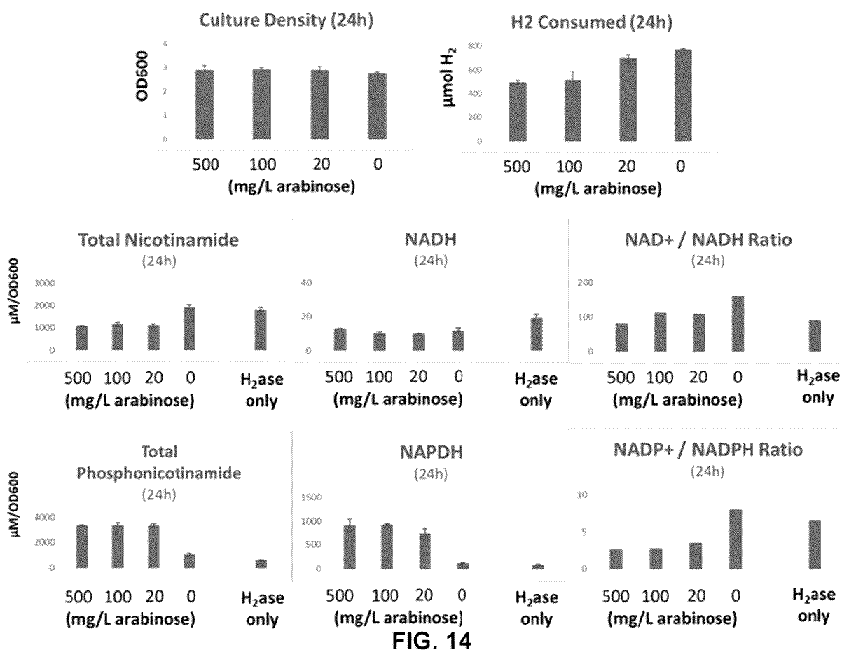

Aspect 7. Both plasmid-based and chromosome-based hydrogenases are functional in BW25113 E. coli. For example, incubating hydrogenase-laden E. coli in the presence of 20% H₂ gas leads to a partial or complete consumption of that gas after an overnight incubation. Gas consumption is monitored indirectly by observing for drops in pressure within sealed vials, or directly by quantifying how much H₂ remains via gas chromatograph. Furthermore, relative to control, H₂ consumption correspondingly leads to increased NADH and NADPH concentration within the host. In some embodiments, introduction of PntAB (or other redox modifying genes, such as NAD kinase) as a third operon of the hydrogenase under control of the tight (such as titratable) arabinose-inducible promoter, combined with the variable effects of origin of replication upon hydrogenase activity (as witnessed during construction of derivative library). We have experimental evidence that the hydrogenase results in an elevation of NADH and NAPDH in E. coli, and that over-expression of PntAB or NAD Kinase elevates NADPH in E. coli. Furthermore, relative to control, H₂ consumption correspondingly leads to increased NADH and NADPH concentration within the host (see FIG. 15).

Aspect 8. Co-expression of hydrogenase and flaviolin polyketide pathways in BW25113 E. coli incubated in LB media led to a 75% improved yield in flaviolin, as compared to a control. As flaviolin does not require NADH and NADPH during its biosynthesis, this experiment demonstrates that the hydrogenase can support biosynthetic pathways that do not require NAD(P)H. The present invention can also be used to support biosynthetic pathways that do require NAD(P)H.

Every example described in the prior literature is the deployment of an external reducing power (e.g., H₂, formate, electricity, etc) to improve a biosynthetic process intentionally chooses processes that require copious NAD(P)H. The key rationale derives from fundamental chemistry: supply abundant external reducing power improves the kinetics of reaction. However, this rationale is not sufficiently comprehensive. Hosts need ATP, NADH, and NADPH to maintain critical cellular processes and to power reductive biochemistry (target molecule of interest). This ATP, NADH, and NADPH can only be derived by oxidizing a portion of your feedstock into CO₂, which decreases the material output of the bioreactor. Aspects 11 and 12 show that H₂ obviates the need of the host to respire significant feedstock into CO₂. The feedstock thus retained is then devoted towards making additional product, resulting in yield improvements. In other words, the flaviolin experiment described herein demonstrates that one can use external reducing power to achieve yield improvements of any product, not merely those that require copious NAD(P)H during its production.

Aspect 9. Kinetic experiments of H₂ consumption in BW25113 E. coli reveal sigmoidal pattern. This could reflect three phases of activity (1) Hydrogenase components are getting transcribed, translated, and post-translationally assembled. Rate of H₂ consumption gradually rises. (2) Cellular demand for reducing power peaks, resulting in maximum rate of H₂ consumption (3) As feedstock is consumed and cells enter stationary phase, cellular demand for reducing power diminishes.

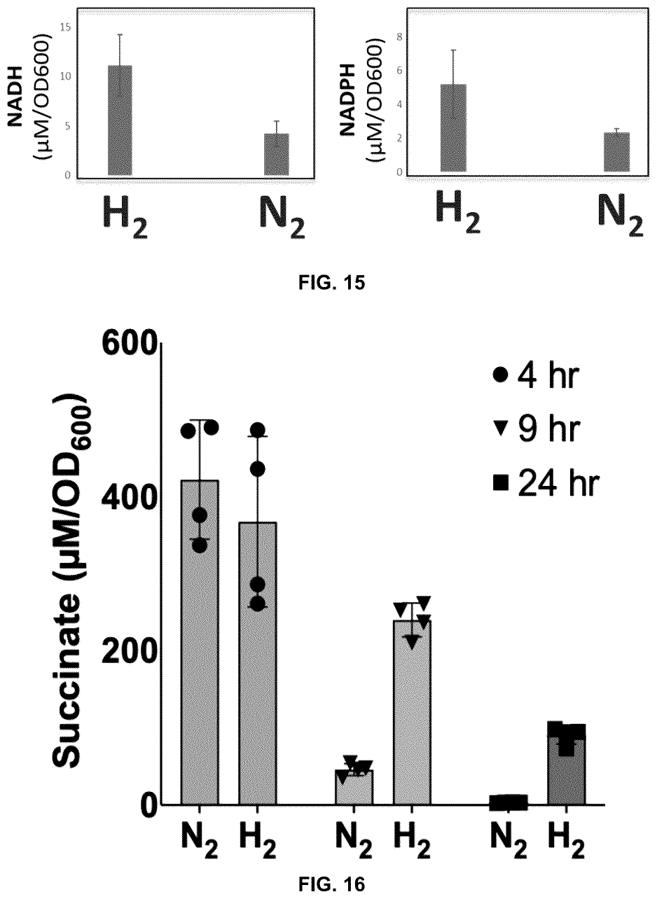

Aspect 10. Incubation of BW25113 E. coli in LB and consumption of H₂ revealed broad metabolic changes. Most poignantly, an enormous elevation in the TCA cycle intermediate succinate. These three metabolites signify activation of the glyoxylate shunt, the non-decarboxylative arm of the TCA cycle. Thus, a specific mechanism of feedstock retention by H₂ is identified. H₂ increases succinate productivity in the E. coli described herein. Further, succinate is a commercially valuable product because it is a precursor chemical in several industrial processes, for example, the production of 1,4-butanediol (the object of Aspect 14). In addition, we demonstrate that the relationship between H₂ consumption and succinate production is titratable (Aspect 11-C). Incubation of BW25113 E. coli in LB and consumption of H₂ revealed broad metabolic changes. Most poignantly, an enormous elevation in TCA cycle intermediates fumarate, succinate, and malate. These three metabolites signify activation of the glyoxylate shunt, the non-decarboxylative arm of the TCA cycle. Thus, a specific mechanism of feedstock retention by H₂ is identified. See FIG. 16.

Aspect 11. When hydrogenase-laden E. coli is grown on acetate as main carbon source, H₂ consumption decreases the amount of acetate feedstock that is consumed. The relationship is titratable. The final cell culture density is identical. The complete oxidation of acetate generates energy that is equivalent to the energy provided by 3.1 moles of H₂. The stoichiometric relationship in our experiment is 1 mole of acetate retained per 3.9 moles of H₂ consumed. It is also observed that less CO₂ is produced, in a fashion that is both titratable to H₂ and with an exact stoichiometry with the amount of acetate retained.

Aspect 12. Using formate and formate dehydrogenase as an NADH-regeneration system has also been demonstrated herein. This further demonstrates that the effects of H₂ are not unique to H₂ but is a generalizable effect of delivering external NADH to a microbial host.

The amino acid sequence of Cupriavidus necator H16 HoxF (NAD-reducing hydrogenase HoxS subunit alpha) is as follows: MDSRITTILERYRSDRTRLIDILWDVQHEYGHIPDAVLPOLGAGLKLSPLDIRETASFYHFFLD KPSGKYRIYLCNSVIAKINGYQAVREALERETGIRFGETDPNGMFGLFDTPCIGLSDQEPAMLI DKVVFTRLRPGKITDIIAQLKQGRSPAEIANPAGLPSQDIAYVDAMVESNVRTKGPVFFRGRTD LRSLLDQCLLLKPEQVIETIVDSRLRGRGGAGFSTGLKWRLCRDAESEQKYVICNADEGEPGTF KDRVLLTRAPKKVFVGMVIAAYAIGCRKGIVYLRGEYFYLKDYLERQLQELREDGLLGRAIGGR AGFDFDIRIQMGAGAYICGDESALIESCEGKRGTPRVKPPFPVQQGYLGKPTSVNNVETFAAVS RIMEEGADWFRAMGTPDSAGTRLLSVAGDCSKPGIYEVEWGVTLNEVLAMVGARDARAVQISGP SGECVSVAKDGERKLAYEDLSCNGAFTIFNCKRDLLEIVRDHMQFFVEESCGICVPCRAGNVDL HRKVEWVIAGKACQKDLDDMVSWGALVRRTSRCGLGATSPKPILTTLEKFPEIYONKLVRHEGP LLPSFDLDTALGGYEKALKDLEEVTR (SEQ ID NO: 1). The homologous variant has a polypeptide sequence that is at least 70%, 75%, 80%, 85%, 90%, 95% or 99% identical to SEQ ID NO: 1, optionally comprises one or more of the amino acid residue(s) at the following position(s): 219-228 (NAD+ binding site), 332-379 (FMN binding site), 499, 502, 505, and/or 545 ([4Fe-4S] cluster binding site), and the hydrogenase retains the enzymatic activity.

The amino acid sequence of Cupriavidus necator H16 HoxU (NAD-reducing hydrogenase HoxS subunit gamma) is as follows: MSIQITIDGKTLTTEEGRTLVDVAAENGVYIPTLCYLKDKPCLGTCRVCSVKVNGNVAAACTVR VSKGLNVEVNDPELVDMRKALVEFLFAEGNHNCPSCEKSGRCQLQAVGYEVDMMVSRFPYRFPV RVVDHASEKIWLERDRCIFCQRCVEFIRDKASGRKIFSISHRGPESRIEIDAELANAMPPEQVK EAVAICPVGTILEKRVGYDDPIGRRKYEIQSVRARALEGEDK (SEQ ID NO: 2). The homologous variant has a polypeptide sequence that is at least 70%, 75%, 80%, 85%, 90%, 95% or 99% identical to SEQ ID NO:2, optionally comprises one or more of the amino acid residue(s) at the following position(s): 35, 46, 49, 61 ([2Fe-2S] binding site), 95, 97, 100, 106 (4Fe-4S] cluster 1 binding site), 145 and/or 148 ([4Fe-4S] cluster 2 binding site), and the hydrogenase retains the enzymatic activity.

The amino acid sequence of Cupriavidus necator H16 HoxY (NAD-reducing hydrogenase HoxS subunit delta) is as follows: MRAPHKDEIASHELPATPMDPALAANREGKIKVATIGLCGCWGCTLSFLDMDERLLPLLEKVTL LRSSLTDIKRIPERCAIGFVEGGVSSEENIETLEHFRENCDILISVGACAVWGGVPAMRNVFEL KDCLAEAYVNSATAVPGAKAVVPFHPDIPRITTKVYPCHEVVKMDYFIPGCPPDGDAIFKVLDD LVNGRPFDLPSSINRYD (SEQ ID NO: 3). The homologous variant has a polypeptide sequence that is at least 70%, 75%, 80%, 85%, 90%, 95% or 99% identical to SEQ ID NO:3, optionally comprises one or more of the cysteine(s) [Fe—S] cluster binding site), and the hydrogenase retains the enzymatic activity.

The amino acid sequence of Cupriavidus necator H16 HoxH (NAD-reducing hydrogenase HoxS subunit beta) is as follows: MSRKLVIDPVTRIEGHGKVVVHLDDDNKVVDAKLHVVEFRGFEKFVQGHPFWEAPMFLQRICGI CFVSHHLCGAKALDDMVGVGLKSGIHVTPTAEKMRRLGHYAQMLOSHTTAYFYLIVPEMLFGMD APPAQRNVLGLIEANPDLVKRVVMLRKWGQEVIKAVFGKKMHGINSVPGGVNNNLSIAERDRFL NGEEGLLSVDQVIDYAQDGLRLFYDFHQKHRAQVDSFADVPALSMCLVGDDDNVDYYHGRLRII DDDKHIVREFDYHDYLDHFSEAVEEWSYMKFPYLKELGREQGSVRVGPLGRMNVTKSLPTPLAQ EALERFHAYTKGRTNNMTLHTNWARAIEILHAAEVVKELLHDPDLQKDQLVLTPPPNAWTGEGV GVVEAPRGTLLHHYRADERGNITFANLVVATTQNNQVMNRTVRSVAEDYLGGHGEITEGMMNAI EVGIRAYDPCLSCATHALGQMPLVVSVFDAAGRLIDERAR (SEQ ID NO: 4). The homologous variant has a polypeptide sequence that is at least 70%, 75%, 80%, 85%, 90%, 95% or 99% identical to SEQ ID NO:4, optionally comprises one or more of the amino acid residue(s) at the following position(s): 62, 65, 458, and/or 461 (Ni²⁺ binding site), and the hydrogenase retains the enzymatic activity.

The amino acid sequence of Cupriavidus necator H16 HoxW is as follows: MNAPAEFPYVTLADFDDPSTLIYGIGNVGRODDGLGWAFIDRLEAESLCSGAEVQRHYQLHLED ADLISRKRKVLFIDATKDASVASFSLERAEPRMDFSFTSHAISIPSIMATCORCFQCLPEVYVL AIRGYEWELRMGLTPQARHNLDDAIAHFSMRAERQTS (SEQ ID NO: 5). The homologous variant has a polypeptide sequence that is at least 70%, 75%, 80%, 85%, 90%, 95% or 99% identical to SEQ ID NO:5, and the hydrogenase retains the enzymatic activity.

The amino acid sequence of Cupriavidus necator H16 HoxI is as follows: MKEQEIDRIATMIYEAPLGEYIGRDGAAILAEHAAEARLLKGDEFLYRRGDVTSSFYIVTDGRL ALVREKTNERTAPI VHVLEKGDLVGELGF IDQTPHSLSVRALGDAAVLSFSAESI KPLITEHPE LIFNFMRAVIKRVHHVVVTVGEHERELQEYISTGGRGRG (SEQ ID NO: 6). The homologous variant has a polypeptide sequence that is at least 70%, 75%, 80%, 85%, 90%, 95% or 99% identical to SEQ ID NO:6, optionally comprises one or more of the amino acid residue(s) at the following position(s): 34-140 (cyclic nucleotide binding), and the hydrogenase retains the enzymatic activity.

The amino acid sequence of Cupriavidus necator H16 HypA2 is as follows: MHEMSLAVGVLQIVEDVAQRDGFSRVTAVRLEIGRLSSIEPEALRFCFEEVVRGSVADGARLEI VDTPGAGWCLHCSETVAIGALYDPCPQCGGYQVQPTGGTEMRVMDLEVA (SEQ ID NO: 7). The homologous variant has a polypeptide sequence that is at least 70%, 75%, 80%, 85%, 90%, 95% or 99% identical to SEQ ID NO:7, and the hydrogenase retains the enzymatic activity.

The amino acid sequence of Cupriavidus necator H16 HypB2 (hydrogenase maturation factor HypB) is as follows: MCTNCGCAAGETRIEGQELEHVDEHAHADGTVHGHAPHHHGEHDHDHGPAYRAVRGTDHLHYGH GPAGAHAPGMSQARMVKIEQDILGKNNAYAAQNRRWFDEHGVFALNFVSSPGSGKTTLLVRTIE ALKSTSRLAVIEGDQQTSFDAERIRATGVQALQINTGKGCHLDAHMVGHALEKLRPEDESVLLI ENVGNLVCPSAFDLGEAHKVVILSVTEGEDKPLKYPDMFRAASLMLLNKCDLLPHLSFDVERAI EYAKRVNPDLHVIRTSSATGEGFDAWLTWIADGLAGOAARRSQSMELLRSRIAGLEAQLAALKV (SEQ ID NO: 8). The homologous variant has a polypeptide sequence that is at least 70%, 75%, 80%, 85%, 90%, 95% or 99% identical to SEQ ID NO:8, optionally comprises one or more of the amino acid residue(s) at the following position(s): 110-265 (CobW/HypB/UreG nucleotide-binding), and the hydrogenase retains the enzymatic activity.

The amino acid sequence of Cupriavidus necator H16 HypF2 (carbamoyltransferase HypF2) is as follows: MLMPRRPRNPRTVRIRIRVRGVVQGVGFRPFVYRLARELGLAGWVRNDGAGVDIEAQGSAAALV ELRERLRRDAPPLARVDEIGEERCAAQVDADGFAILESSRSDAAVHTAIGHDTAVCPDCLAELF DPANRRYRYAFINCTQCGPRYTLTWALPYDRATTSMAPFPQCRPCLDEYNAPEHRRFHAEPNAC PDCGPSLALLNAQGMPVEDVDPIAETVARLORGEIVAIKGLGGFHLACDAHNADAVARLRSRKQ REEKPFAVMVANLATAAQWGDIGSGEAALLTASERPIVLLRKRSGVDGRFAGVAPGLVWLGVML PYTPLQYLLFHEAAGRPEGLGWLAQPQSLVLVMTSANPGGEPLVTGNDEAAQRLTGIADAFLLH DREILVRCDDSVVRGDGEPAPHVQFIRRARGYTPRAIKLARSGPSVLALGGSFKNTVCLTRGDE AFVSQHVGDLGNAATCEALIEAVAHLQRVLEIRPQLVAHDLHPDFFSTRHAAELAAQWGVPAVA VQHHHAHIAAVLAEHGSDEPAIGLALDGVGLGDDGQAWGGELLLVDGGACKRLGHLRELPLPGG DRAAREPWRMAAAALHAMGRGEEIEGRFPRQPGAPMVNRMLAQRLNAPLSSSMGRWFDAAAGLL GTRETMAYEGQAAMLLEGLAESWGEQPSPGRPKTVAHSLGGVPRSGGGTYKALALPDAWRIDAG NTLDLLPLLEALSAETNAARGAAQFHATLVAALEAWTVATVQVTGVRTVVFGGGCFLNHILARN LCRRLAARGLTVLTARQLPPNDGGIALGOVWVALORAPN (SEQ ID NO: 9). The homologous variant has a polypeptide sequence that is at least 70%, 75%, 80%, 85%, 90%, 95% or 99% identical to SEQ ID NO:9, optionally comprises one or more of the amino acid residue(s) at the following position(s): 14-101 (acylphophastase-like), 120-145 (C4-type zinc finger), 170-195 (C4-type zinc finger), and/or 212-415 (YrdC-like), and the hydrogenase retains the enzymatic activity.

The amino acid sequence of Cupriavidus necator H16 HypC1 is as follows: MCLAIPARLVELQADQQGVVDLSGVRKTISLALMADAVVGDYVIVHVGYAIGKIDPEEAERTLR LFAELERVQPPASEPMHGMNIHQEPA (SEQ ID NO: 10). The homologous variant has a polypeptide sequence that is at least 70%, 75%, 80%, 85%, 90%, 95% or 99% identical to SEQ ID NO: 10, and the hydrogenase retains the enzymatic activity.

The amino acid sequence of Cupriavidus necator H16 HypD1 (hydrogenase maturation factor HypD) is as follows: MKYIEEFRDGELAQRIAAHVRAEARPGORYNFMEFCGGHTHAISRYGVTELLPENVRMIHGPGC PVCVLPIGRIDLALHLALERDAIVCTYGDTMRVPASGGMSLIRAKAHGADIRMVYSAADALKIA QRHPQREVVFLAIGFETTTPPTALIIREAKARQVDNFSVLCCHVLTPSAITHILESPEVRDYGT VPIDGFVGPAHVSIVIGTRPYEHFSREYGKPVVIAGFEPLDVMQAILMLVRQVNSGRAEVENEF VRAVTRDGNESAQAMVSEVFELRPSFEWRGLGEVPYSALRIRAQFARFDAEQRFDLRYRPVPDN KACECGAILRGVKKPTDCKLFATVCTPENPMGSCMVSSEGACAAHYSYGRFKDIPLVAA (SEQ ID NO: 11). The homologous variant has a polypeptide sequence that is at least 70%, 75%, 80%, 85%, 90%, 95% or 99% identical to SEQ ID NO:11, optionally comprises one or more of the amino acid residue(s) at the following position(s): 36, 64, and/or 67 (Fe cation binding site), and the hydrogenase retains the enzymatic activity.

The amino acid sequence of Cupriavidus necator H16 HypE1 is as follows: MSGTVKLGYQRPLNIKSGRIDMGHGAGGRAAAQLIQELFVAAFDNEWLRQGNDQAAFAMPAGAR MVMATDAHVVSPLFFPGGDIGSLSVHGTINDVAMAGAKPLYLAASFILEEGFPLADLKRIVESM AGAAREAGVPIVTGDTKVVEQGKGDGVFITTTGVGVVPAGILIDGAGARPGDAILLSGTMGEHG VAILSKRESLEFDTEIRSDSAALHDLVAQMLAVVPGVRVLRDPTRGGLATTLNEISSQSGVGMV LDEAAIPVLPQVDAACELLGLDPLYVANEGKLVAICAAADADALLAAMRGHPLGREARRIGEVI EDGRHFVQMRTKFGGMRVVDWLSGEQLPRIC (SEQ ID NO: 12). The homologous variant has a polypeptide sequence that is at least 70%, 75%, 80%, 85%, 90%, 95% or 99% identical to SEQ ID NO:12, and the hydrogenase retains the enzymatic activity.

The amino acid sequence of Cupriavidus necator H16 HypX is as follows: MRILLLTHSFNSLTQRLFVELRQRGHLVSVEFDIADSVTEEAVALFAPDLVIAPFLKRAIPERI WSRLVCLVVHPGIVGDRGPSALDWAIVRDERSWGVTVLQANGEMDAGPVWASATFPMRAARKSS LYRNEVTVAAVQAVLEALAAFEAGWRSANDAQSGPGTWNPAMRQAERGIDWARESTAAVLAKLH AADSFPGVPDALFGQPCRLFDAHAATPQTVARAPRGQPGDIVARREHAVLRLTVDAGVWIGHAK LAVRDEWENTSLPSLKLPVAQVFHEAWAQLPDLSLPLEHDAGEWSEFRYWEDDSGAGLVGYLAF DCYNGAMSTAQCQRLCAALRYARGRQTRVLVLLGGEDFFSNGIHLHQIEAAEHRGAESAADASW RNIQAMDDVALEILAFSDRMTVSALRGNAGAGGVFLALAADQVWAREGVLVNPHYKNMGNLYGS EYWTYLLPQRVGAQRASDLMDGRLPMSVRRAIEIGLIDASLDGDARSCLAEIGRRAVALARAPD YAAHIDNKRRKRAADEAAKPLAQYREEELVHMHRNFYGFDPSYHVARYHFVYKLPHAHTPRHLA MHRGGSLPAAQELQALAGKRS (SEQ ID NO: 13). The homologous variant has a polypeptide sequence that is at least 70%, 75%, 80%, 85%, 90%, 95% or 99% identical to SEQ ID NO: 13, optionally comprises one or more of the amino acid residue(s) at the following position(s): 39-145 (formyl transferase N-terminal), and the hydrogenase retains the enzymatic activity.

### REFERENCES CITED

1. Zhang Y H P, Sun J,Ma Y. 2017. Biomanufacturing: History and perspective. J. Ind. Microbiol. Biotechnol. 44:773-784
2. Kung Y, Runguphan W, Keasling J D. 2012. From fields to fuels: Recent advances in the microbial production of biofuels. ACS Synth. Biol. 1:498-513.
3. Pearsall S M, Rowley C N, Berry A. 2015. Advances in pathway engineering for natural product biosynthesis. ChemCatChem 7:3078-3093
4. Adom F, et al. 2014. Life-cycle fossil energy consumption and greenhouse gas emissions of
5. bioderived chemicals and their conventional counterparts. Environ. Sci. Technol. 48:14624-14631.
6. Deng Y, Beahm D R, Ionov S, Sarpeshkar R. 2021. Measuring and modeling energy and power consumption in living microbial cells with a synthetic ATP reporter. BMC Biol. 19:101
7. Cruz-Moralez P, Yin K, Landera A, Cort J R, Young R P, Kyle J E, Bertrand R, Iavarone A T, et al. 2022. Biosynthesis of polycyclopropanated high energy biofuels. Joule 6:1590-1605
8. Bar-Even A, Flamholz A, Noor E, Milo R. 2012. Thermodynamic constraints shape the structure of carbon fixation pathways. Biochim. Biophys. Acta. 1817:1646-1659
9. Flamholz A, Noor E, Bar-Even A, Liebermeister W, Milo R. 2013. Glycolytic strategy as a tradeoff between energy yield and protein cost. Proc. Natl. Acad. Sci. U.S.A. 110:10039-10044.
10. Noor E, Bar-Even A, Flamholz A, Reznik E, Liebermeister W, Milo R. 2014. Pathway thermodynamics highlights kinetic obstacles in central metabolism. PLoS Comput. Biol. 10: e1003483
11. Du B, Zielinski D C, Monk J M, Palsson B O. 2018. Thermodynamic favorability and pathway yield as evolutionary tradeoffs in biosynthetic pathway choice. Proc. Natl. Acad. Sci. U.S.A. 115:11339-11344.
12. Yu H, et al. 2018a. Augmenting the Calvin-Benson-Bassham cycle by a synthetic malyl-CoA-glycerate carbon fixation pathway. Nat. Commun. 9:2008
13. Bogorad, I. W. et al. 2014. Building carbon-carbon bonds using a biocatalytic methanol condensation cycle. Proc. Natl. Acad. Sci. USA 111, 15928-15933
14. Ng C Y, Farasat I, Maranas C D, Salis H M. 2015. Rational design of a synthetic Entner-Doudoroff pathway for improved and controllable NADPH regeneration. Metab. Eng. 29:86-96
15. Clomburg J M, Qian S, Tan Z, Cheong S, Gonzalez R. 2019. The isoprenoid alcohol pathway, a synthetic route for isoprenoid biosynthesis. Proc. Natl. Acad. Sci. U.S.A. 116:12810-12815
16. Yu H, Liao J C. 2018. A modified serine cycle in Escherichia coli coverts methanol and CO₂ to two-carbon compounds. Nat. Commun. 9:3992
17. Yang J, Nie Q, Liu H, Xian M, Liu H. 2016. A novel MVA-mediated pathway for isoprene production in engineered E. coli. BMC Biotechnol. 16:5
18. Kang A, George K W, Wang G, Baidoo E, Keasling J D, Lee T S. 2016. Isopentenyl diphosphate (IPP)-bypass mevalonate pathway for isopentenol production. Metab. Eng. 34:25-35.
19. Bang J, Lee S Y. 2018. Assimilation of formic acid and CO₂ by engineered Escherichia coli equipped with reconstructed one-carbon assimilation pathways. Proc. Natl. Acad. Sci. U.S.A. 115: E9271-E9279
20. Antonovsky N, Gleizer S, Noor E, Zohar Y, Herz E, et al. 2016. Sugar Synthesis from CO₂ in Escherichia coli. Cell 166:115-125 Incorporated fully functional CBB cycle in E. coli
21. Guadalupe-Medina V, Wisselink H W, Luttik M A, de Hulster E, Daran J-MG, Pronk J T, van Maris A J A. Carbon dioxide fixation by Calvin-cycle enzymes improves ethanol yield in yeast. Biotechnol Biofuels. 2013; 6:125.
22. Chen Y, Gin J W, Wang Y, de Raad M, Tan S, Jillson N J, Northen T R, Adams P D, Petzold C J. 2023. Alkaline-SDS cell lysis of microbes with acetone protein precipitation for proteomic sample preparation in 96-well plate format. PLoS ONE 18: e0288102
23. Chen Y, Gin J, Petzold C J. 2022. Discovery proteomic (DIA) LC-MS/MS data acquisition and analysis V.2. protocols.io dx.doi.org/10.17504/protocols.io.e6nywk1z7vmk/v2
24. Zhuang Z Y, Li S Y. 2013. Rubisco-based engineering Escherichia coli for in situ carbon dioxide recycling. Bioresource Technology 150:79-88
25. Gleizer S, Ben-Nissan R, Bar-On Y M, Antonovsky N, Noor E, Zohar Y, Jona G, Krieger E, Shamshoum M, Bar-Even A, Milo R. 2019. Conversion of Escherichia coli to generate all biomass carbon from CO₂. Cell 179:1255-1263.
26. Lin P P, Jaeger A J, Wu T Y, Xu S C, Lee A S, Gao F, Chen P W, Liao J C. 2018. Construction and evolution of an Escherichia coli strain relying on nonoxidative glycolysis for sugar catabolism. Proc. Natl. Acad. Sci. U.S.A. 115:3538-3546.
27. Tashiro Y, Desai S H, Atsumi S. 2015. Two-dimensional isobutyl acetate production pathways to improve carbon yield. Nat. Commun. 6:7488
28. Meadows A L, Hawkins K M, Tsegaye Y, Antipov E, et al. 2016. Rewriting yeast central carbon metabolism for industrial isoprenoid production. Nature 537:694-697
29. Lonsdale T H, Lauterbach L, Malca S H, Nestl B M, Hauer B, Lenz O. 2015. H₂-driven biotransformation of n-octane to 1-octanol by a recombinant Pseudomonas putida strain co-synthesizing an O₂-tolerant hydrogenase and a P450 monooxygenase. 51:16173-16175
30. Millard P, Enjalbert B, Uttenweiler-Joseph S, Portais J C, Létisse F. 2021. Control and regulation of acetate overflow in Escherichia coli. eLife 10: e63661
31. Litsios A, Ortega A D, Wit E C, Heinemann M. 2018. Metabolic-flux dependent regulation of microbial physiology. Curr. Opin. Microbiol. 42:71-78.
32. Holm A K, Blank L M, Oldiges M, Schmid A, Solem C, Jensen P R, Vemuri G N. 2010. Metabolic and transcriptional response to cofactor perturbations in Escherichia coli. J. Chem. Biol. 285:17498-17506.
33. Martín AJ, Larrazábal GO, Pérez-Ramírez J. 2015. Towards sustainable fuels and chemicals through the electrochemical reduction of CO₂: Lessons from water electrolysis. Green Chem. 17:5114-5130
34. Nybo S E, Khan N E, Woolston B M, Curtis W R. 2015. Metabolic engineering in chemolithoautotrophic hosts for the production of fuels and chemicals. Metab. Eng. 30:105-120.
35. Liu C, Colón BC, Ziesack M, Silver P A, Nocera D G. 2016. Water splitting-biosynthetic system with CO₂ reduction efficiencies exceeding photosynthesis. Science 352:1210-1213
36. Levi P G, Cullen J M. 2018. Mapping global flows of chemicals: From fossil fuel feedstocks to chemical products. Environ. Sci. Technol. 52:1725-1734.
37. Moore A. 2008a. Biofuels are dead: Long live biofuels (?)—Part One. New Biotechnol. 25:6-12.
38. Moore A. 2008b. Biofuels are dead: Long live biofuels (?)—Part Two. New Biotechnol. 25:96-100.
39. Zheng T, Li J, Ji Y, Zhang W, Fang Y, Xin F, Dong W, Wei P, Ma J, Jiang M. 2020. Progress and prospects of bioelectrochemical systems: Electron transfer and its applications in the microbial metabolism. Front. Bioeng. Biotechnol. 8:
40. Yamada S, Takamatsu Y, Ikeda S, Kouzuma A, Watanabe K. 2022. Towards application of electro-fermentation for the production of value-added chemicals from biomass feedstocks. Front. Chem. 9:
41. Yishai O, Lindner S N, de la Cruz J G, Tenenboim H, Bar-Even A. 2016. The formate bio-economy. Curr. Opin. Chem. Biol. 35:1-9.
42. Cotton C A R, Claassens N J, Benito-Vaquerizo S, Bar-Even A. 2020. Renewable methanol and formate as microbial feedstocks. Curr. Opin. Biotechnol. 62:168-180
43. Partipilo M, Claassens N J, Slotboom D J. 2023. A hitchhiker's guide to supplying enzymatic reducing power into synthetic cells. ACS Synth. Biol. 12:947-962.
44. Claassens N, Sánchez-Andrea I, Sousa D Z, Bar-Even A. 2018. Towards sustainable feedstocks: A guide to electron donors for microbial carbon fixation. Curr. Opin. Biotechnol. 50:195-205.
45. Wang Y, San K Y, Bennett G N. 2013. Cofactor engineering for advancing chemical biotechnology. Curr. Opin. Biotechnol. 24:994-999
46. Thakker C, Martínez I, Li W, San K Y, Bennett G N. 2015. Metabolic engineering of carbon and redox flow in the production of small organic acids. J. Ind. Microbiol. Biotechnol. 42:403-422.
47. Lauterbach L, Lenz O. 2019. How to make the reducing power of H₂ available for in vivo biosyntheses and biotransformation. Curr. Opin. Chem. Biol. 49:91-96.
48. Ji M, Wang J. 2019. Review and comparison of various hydrogen production methods based on costs and life cycle impact assessment indicators. Int. J. Hyd. Energy 46:38612-38635.
49. Lubitz W, Ogata H, Rüdiger O, Reijerse E. 2014. Hydrogenases. Chem. Rev. 114:4081-4148
50. Lauterbach L, Liu J, Horch M, Hummel P, Schwarze A, Haumann M, Vincent K A, Lenz O, Zebger I. 2011a. The hydrogenase subcomplex of the NAD⁺-reducing [NiFe] hydrogenase from Ralstonia eutropha—Insights into catalysis and redox interconversions. Eur. J. Inorg. Chem. 2011:1067-1067.
51. Lauterbach L, Idris Z, Vincent K A, Lenz O. 2011b. Catalytic properties of the isolated diaphorase fragment of the NAD⁺-reducing [NiFe]-hydrogenase from Ralstonia eutropha. PLoS One 6: e25939.
52. English C M, Eckert C, Brown K, Seibert M, King P W. 2009. Recombinant and in vitro expression systems for hydrogenases: New frontiers in basic and applied studies for biological and synthetic H₂ production. Dalton Trans. 2009:9970-9978
53. Fan Q, Neubauer P, Lenz O, Gimpel M. 2020. Heterologous hydrogenase overproduction systems for biotechnology—An overview. Int. J. Mol. Sci. 21:5890
54. Panich J, Fong B, Singer S W. 2021. Metabolic engineering of Cupriavidus necator H16 for sustainable biofuels from CO₂. Trends Biotechnol. 39:412-424
55. Zhang L, Jiang Z, Tsui T H, Loh K C, Dai Y, Tong Y W. 2022. A review on enhancing Cupriavidus necator fermentation for poly(3-hydroxybutyrate) (PHB) production from low-cost carbon sources. Front Bioeng. Biotechnol. 10:946085.
56. Parkin A, Sargent F. 2012. The hows and whys of aerobic H₂ metabolism. Curr. Opin. Chem. Biol. 16:26-34.
57. Pandelia M E, Bykov D, Izsak R, Infossi P, Giudici-Orticoni M T, Bill E, Neese F, Lubits W. 2012. Electronic structure of the unique [4Fe-3S] cluster in O₂-tolerant hydrogenases characterized by ⁵⁷Fe Mössbauer and EPR spectroscopy. Proc. Natl. Acad. Sci. U.S.A. 110:483-488
58. Lauterbach L, Lenz O. 2013. Catalytic production of hydrogen peroxide and water by oxygen-tolerant [NiFe]-hydrogenase during H₂ cycling in the presence of O₂. J. Am. Chem. Soc. 135:17897-17905.
59. Shafaat, H. et al. (2013) [NiFe] hydrogenases: a common active site for hydrogen metabolism under diverse conditions. Biochim Biophys Acta 1827, 986-1002
60. Lu Y, Koo J. 2019. O₂ sensitivity and H₂ production activity of hydrogenases—A review. Biotech. Bioeng. 116:3124-3135.
61. Ghosh, Bisaillon, Hallenbeck (2013) Increasing the metabolic capacity of Escherichia coli for hydrogen production through heterologous expression of the Ralstonia eutropha SH operon. Biotechnol. Biofuels 6:122
62. Teramoto H, Shimizu T, Suda M, Inui M. 2022. Hydrogen production based on the heterologous expression of NAD⁺-reducing [NiFe]-hydrogenase from Cupriavius necator in different genetic backgrounds of Escherichia coli strains. Int. J. Hydrogen Energy 47:22010-22021.
63. Lamont, Sargent. (2017). Design and characterization of synthetic operons for biohydrogen technology. Arch. Microbiol. 199:495-503.
64. Schiffels, Pinkenburg, Schelden, Aboulnaga, Baumann, Selmer. (2013) An innovative cloning platform enables large-scale production and maturation of an oxygen-tolerant [NiFe]-hydrogenase from Cupriavidus necator in Escherichia coli. PLoS ONE 8: e68812
65. Fan Q, Caserta G, Lorent C, Zebger I, Neubauer P, Lenz O, Gimpel M. 2022. High-yield production of catalytically active regulatory [NeFe]-hydrogenase from Cupriavidus necator into Escherichia coli. Front. Microbiol. 13:894375
66. Bosdriesz E, Molenaar D, Tuesink B, Bruggeman F J. 2015. How fast-growing bacteria robustly tune their ribosome concentration to approximate growth-rate maximization. FEBS J. 282:2029-2044.
67. Molenaar D, van Berlo R, de Ridder D, Teusink B. 2009. Shifts in growth strategies reflect tradeoff in cellular economics. Mol. Syst. Biol. 5:323.
68. Vogel C, Marcotte E M. 2012. Insights into the regulation of protein abundance from proteomic and transcriptomic analyses. Nat. Rev. Genet. 13:227-232.
69. Batth T S, Keasling J D, Petzhold C J. 2012. Targeted proteomics for metabolic pathway optimization. Methods Mol. Biol. 944:237
70. Alonso-Gutierrez J, Kim E M, Batth T S, Cho N, Hu Q, Chan L J G, Petzhold C J, Hillson N J, Adams P D, Keasling J D, Martin H G, Lee T S. 2015. Principal component analysis of proteomics (PCAP) as a tool to direct metabolic engineering. Metab. Eng. 28:123-133.
71. Petzold C J, Chan L J G, Nhan M, Adams P D. 2015. Analytics for metabolic engineering. Front. Bioeng. Biotechnol. 3:135
72. Jahn M, Crang N, Janasch M, Hober A, Forsstrom B, Kimler K, Mattausch A, Chen Q, Asplund-Samuelsson J, Hudson E P. 2021. Protein allocation and utilization in the versatile chemolithoautotrophy Cupriavidus necator. Elife 10: e69019.
73. Ham T S, Dmytriv Z, Plahar H, Chen J, Hillson N J, Keasling J D. 2012. Design, implementation and practice of JBEI-ICE: An open source biological part registry platform and tools. Nucl. Acids. Res
74. Kwok R (2010) Five hard truths for synthetic biology. Nature 463:288-290
75. Shetty R P, Endy D, Knight T F Jr. 2008. Engineering BioBrick vectors from BioBrick parts. J. Biol. Eng. 2:5
76. Lee T S, Krupa R A, Zhang F, Hajimorad M, Holtz W J, Prasad N, Lee S K, Keasling J D. 2011. BglBrick vectors and datasheets: A synthetic biology platform for gene expression. J. Biol. Eng. 5:12.
77. Yang D, Kim W J, Yoo S M, Choi J H, Ha S H, Lee M H, Lee S Y. 2018. Repurposing type III polyketide synthase as a malonyl-CoA biosensor for metabolic engineering in bacteria. Proc Nat Acad Sci. USA 115:9835-9844
78. LeBlanc IV CH. Scale Up of a Novel Method to Maximize Malonyl-CoA in Escherichia Coli. Master's Thesis, Louisiana State University, 2022.
79. Orr J S, Christensen D G, Wolfe A J, Rao C V. 2019. Extracellular acidic pH inhibits acetate consumption by decreasing gene transcription of the tricarboxylic acid cycle and the glyoxylate shunt. J. Bacteriol. 201: e00410-18.
80. Steigmiller S, Turina P, Gräber P. 2008. The thermodynamic H+/ATP ratios of the H+-ATPsynthases from chloroplasts and Escherichia coli. Proc. Natl. Acad. Sci. USA 105:3745-3750.
81. Berry S, Rumberg B. 1996. H⁺/ATP coupling ratio at the unmodulated CF₀F₁-ATP synthase determined by proton flow measurements. Biochim. Biophys. Acta 1276:51-56.
82. Van Walraven H S, Strotmann H, Schwarz O, Rumberg B. 1996. The H⁺/ATP coupling ratio of the ATP synthase from thiol-modulated chloroplasts and two cyanobacterial strains is four. FEBS Lett. 379:309-313.
83. Turina P, Samoray D, Gräber P. 2003. H⁺/ATP ratio of proton transport-coupled ATP synthesis and hydrolysis catalysed by CF₀F₁-liposomes. EMBO J. 22:418-426
84. Petersen J, Förster K, Turina P, Gräber P. 2012. Comparison of the H⁺/ATP ratios of the H⁺-ATP synthases from yeast and from chloroplast. Proc. Natl. Acad. Sci. U.S.A. 109:11150-5.
85. Tomashek J J, Brusilow W S. 2000. Stoichiometry of energy coupling by proton-translocating ATPases: A history of variability. J Bionenerg Biomembr 32:493-500
86. Chapman B, Loiselle D. 2016. Thermodynamics and kinetics of the F₀F₁-ATPase: Application of the probability isotherm. R. Soc. Open. Sci. 3:150379.
87. Fergunson S J. 2010. ATP synthase: From sequence to ring size to the P/O ratio. Proc. Natl. Acad. Sci. U.S.A. 107:16755-16756
88. Turina P, Petersen J, Gräber P. 2016. Thermodynamics of proton transport coupled ATP synthesis. Biochim. Biophys. Acta 1857:653-664
89. Balzer G J, Thakker C, Bennett G N, San K Y. 2013. Metabolic engineering of Escherichia coli to minimize byproduct formate and improving succinate productivity through increased NADH availability by heterologous expression of NAD⁺-dependent formate dehydrogenase. Metab. Eng. 20:1-8.
90. Wolfe A J. 2005. The acetate switch. Microbiol. Mol. Biol. Rev. 69:12-50
91. Loferer-Kröβbacher M, Klima J, Psenner R. 1998. Determination of bacterial cell dry mass by transmission electron microscopy and densitometric image analysis. Appl Environ Microbiol. 64:688-94
92. Heldal M, Norland S, Tumyr O. 1985. X-ray microanalytic method for measurement of dry matter and elemental content of individual bacteria. Appl. Environ. Microbiol. 50:1251-1257
93. Fagerbakke K M, Heldal M, Norland S. 1996. Content of carbon, nitrogen, oxygen, sulfur and phosphorus in native aquatic and cultured bacteria. Aquat. Microb. Ecol. 10:15-27
94. Milo R, Phillips R. 2015. Cell Biology by the Numbers. Garland Science, New York. Webpage for: doi.org/10.1201/9780429258770
95. Link K, Anselment B, Weuster-Botz D W. 2008. Leakage of adenylates during cold methanol/glycerol quenching of Escherichia coli. Metabolomics 4:240-247
96. Shiloach J, Fass R. 2005. Growing E. coli to high cell density—a historical perspective on method development. Biotechnol. Adv. 23:345-357
97. Ji M, Wang J. 2021. Review and comparison of various hydrogen production methods based on costs and life cycle impact assessment indicators. Int. J. Hydrogen Energy 46:38612-38635.
98. Pivovar B S, Ruth M F, Myers D J, Dinh H N. 2021. Hydrogen: Targeting $1/kg in 1 Decade. Electrochem. Soc. Interface 30:61
99. Merfort L, Bauer N, Humpenoder F, Klein D, Strefler J, Popp A, Luderer G, Kriegler E. 2023. State of global land regulation inadequate to control biofuel land-use-change emissions. Nature Climate Change 13:610-612.
100. De Luna P, Hahn C, Higgins D, Jaffer S A, Jaramillo T F, Sargent E H. 2019. What would it take for renewably powered electrosynthesis to displace petrochemical processes? Science 364: eaav3506
101. Spurgeon J M, Kumar B. 2018. A comparative technoeconomic analysis of pathways for commercial electrochemical CO₂ reduction to liquid products. Energy Environ. Sci. 11:1536-1551.
102. International Renewable Energy Agency, Renewable Power: Climate-Safe Energy Competes on Cost Alone, 2018
103. Parag Y, Sovacool B K. 2016. Electricity market design for the prosumer era. Nat. Energy 1:16032
104. Keith D W, Holmes G, St. Angelo D, Heidel K. 2018. A process for capturing CO₂ from the atmosphere. Joule 2:1573-1594
105. Singh A K, Singh S, Kumar A. 2016. Hydrogen energy future with formic acid: A renewable chemical hydrogen storage systems. Cata. Sci. Technol. 6:12-40.
106. Eppinger J, Huang K W. 2017. Formic acid as a hydrogen energy carrier. ACS Energy Lett. 2:188-195
107. Huang Y X, Hu Z. 2018. An integrated electrochemical and biochemical system for sequential reduction of CO₂ to methane. Fuel 220:8-13.

108. Connelly T. M., Jr., Change M. C., Clarke L., Hillson N. J., Johnson R. A., Keasling J. D., et al. (2015). Industrialization of Biology: A Roadmap to Accelerate the Advanced Manufacturing of Chemicals. Washington, DC: National Research Council, 10.17226/19001
109. Werpy T, Petersen G. 2004. Top Value Added Chemicals from Biomass: Volume I—Results of Screening for Potential Candidates from Sugars and Synthesis Gas. United States Department of Energy (DoE). Webpage for: doi.org/10.2172/15008859
110. Zhu L W, Tang Y J. 2017. Current advances of succinate biosynthesis in metabolically engineered Escherichia coli. Biotechnol Adv. 35:1040-1048
111. Claassens N J, Cotton C A R, Kopljar D, Bar-Even A. 2019. Making quantitative sense of electromicrobial production. Nat. Catal. 2:437-447.
112. Teramoto H, Shimizu T, Suda M, Inui M. 2022. Hydrogen production based on the heterologous expression of NAD⁺-reducing [NiFe]-hydrogenase from Cupriavidas necator in different genetic backgrounds of Escherichia coli. Int. J. Hydrogen Energy 47:22010-22021.
113. Maeda T, Vardar G, Self W T, Wood T K. 2007. Inhibition of hydrogen uptake in Escherichia coli by expressing the hydrogenase from the cyanobacterium Synechocystis sp. PCC 6803. BMC Biotechnol. 7:25
114. Jo B H, Cha J H. 2015. Activation of formate hydrogen-lyase via expression of uptake [NiFe]-hydrogenase in Escherichia coli BL21 (DE3). Microb. Cell Fact. 14:151
115. Dürre P. 2017. Gas fermentation—a biotechnological solution for today's challenges. Microb. Biotechnol. 10:14-16.
116. Nangle S N, et al. 2020. Valorization of CO₂ through lithoautotrophic production of sustainable chemicals in Cupriavidus necator. bioRxiv 2020:1-42.
117. Tollefson J. 2008. Energy: Not your father's biofuels. Nature 451:880-883
118. Takors R, Kopf M, Mampel J, Bluemke W, et al. 2018. Using gas mixtures of CO, CO₂ and H₂ as microbial substrates: The do's and don'ts of successful technology transfer from laboratory to production scale. Microb. Biotechnol. 11:606-625
119. Qi M, Park J, Landon R S, Kim J, Liu Y, Moon I. 2022. Continuous and flexible renewable-power-to-methane via liquid CO₂ energy storage: Revisiting the technoeconomic potential. Renewable and Sustainable Energy Reviews 153:111732.
120. Angelidaki I, Treu L, Tsapekos P, Luo G, Campanaro S, Wenzel H, Kougias P G. 2018. Biogas upgrading and utilization: Current status and perspectives. Biotechnol. Adv. 36:452-466
121. Al-Shameri A, Willot S J P, Paul C E, Hollmann F, Lauterbach L. 2020. H₂ as a fuel for flavin- and H₂O₂-dependent biocatalytic reactions. Chem. Commun. 56:9667-9670
122. Yang D, Cho J S, Choi K R, Kim H U, Lee S Y. 2017. Systems metabolic engineering as an enabling technology in accomplishing sustainable development goals. Microbial Biotechnol. 10:1254-1258
123. Fagunwa, O. E., and Olanbiwoninu, A. A. (2020). Accelerating the Sustainable Development Goals through Microbiology: Some Efforts and Opportunities. Access Microbiol. 2,
124. Bengelsdorf F R, Dürre P. 2017. Gas fermentation for commodity chemicals and fuels. Microb. Biotechnol. 10:1167-1170
125. Amer B, Kakumanu R, Baidoo E E K. 2022. HILIC-MS analysis of central carbon metabolites in gram negative bacteria. (Protocols.io dx.doi.org/10.17504/protocols.io.4r312opzxv1y/v1)
126. Ahrné E, Molzahn L, Glatter T, Schmidt A. 2013. Critical assessment of proteome-wide label-free absolute abundance estimation strategies. Proteomics 13:2567-2578.
127. Silva J C, Gorenstein M V, Li G Z, Vissers J P C, Geromanos S J. 2006. Absolute quantification of proteins by LCMSE: A virtue of parallel MS acquisition. Mol. Cell Proteomics 5:144-156.
128. Wenk S. et al. 2020. An “energy-auxotroph” Escherichia coli provides an in vivo platform for assessing NADH regeneration systems. Biotechnol. Bioeng. 117:3422-3434.
129. Sauer U, Canonaco F, Heri S, Perrenoud A, Fischer E. 2004. The soluble and membrane-bound transhydrogenases UdhA and PntAB have divergent functions in NADPH metabolism of Escherichia coli. J. Biol. Chem. 279:6613-6619.
130. Whipple, D. and Kenis, J. (2010) Prospects of CO₂ utilization via direct heterogenous electrochemical reduction. J Phys Chem Lett 1, 3451-3458
131. Schneider, K. and Schlegel, H. (1976) Purification and properties of soluble hydrogenase from Alcaligenes eutrophus H16. Biochim. Biophys. Acta 452, 66-80
132. Horch, M. et al. (2010) Probing the active site of an O2-tolerant NAD(+)-reducing [NiFe]-hydrogenase from Ralstonia eutropha H16 by in situ EPR and FTIR spectroscopy. Angew Chem Int 49, 8026-8029
133. Burgdorf, T. et al. (2005) Structural and oxidation-state changes at its nonstandard Ni—Fe site during activation of the NAD-reducing hydrogenase from Ralstonia eutropha detected by X-ray absorption, EPR, and FTIR spectroscopy. J Am Chem Soc 127, 576-592
134. Happe, R. et al. (2000) Unusual FTIR and EPR properties of the H2-activating site of the cytoplasmic NAD-reducing hydrogenase from Ralstonia eutropha. FEBS Lett 466, 259-263
135. Van der Linden, E. et al. (2004) The soluble [NiFe]-hydrogenase from Ralstonia eutropha contains four cyanides on its active site, one of which is responsible for the insensitivity towards oxygen. J Biol Inorg Chem 9, 616-626
136. Thiemermann, S. et al. (1996) Carboxyl-terminal processing of the cytoplasmid NAD-reducing hydrogenase of Alcaligenes eutrophus requires the hoxW gene product. J Bacteriol 178, 2368-2374
137. Burgdorf, T. et al. (2005) The soluble NAD+-reducing [NiFe]-hydrogenase from Ralstonia eutropha H16 consists of six subunits and can be specifically activated by NADPH. J Bacteriol 187, 3122-3132
138. Lupacchini, S. et al. (2021) Rewiring cyanobacterial photosynthesis by the implementation of an oxygen-tolerant hydrogenase. Metab. Eng. 68, 199-209
139. Iizasa, E. and Nagano, Y. (2006) Highly efficient yeast-based in vivo DNA cloning of multiple DNA fragments and the simultaneous construction of yeast/Escherichia coli shuttle vectors. Biotechniques 40, 79-83

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### External Electron Sources Enables Efficient and Decarbonized Biomanufacturing

Industrial microbiology offers a clean and renewable future, but most applications are economically uncompetitive with petroleum. Conventional practice dictates that feedstock is used as both the substrate for bioconversion and the source of energy. On a cost-per-energy basis, H₂ can provide at least three times more cellular energy to microbes than every digestible feedstock including wholesale sucrose. Generalizable cost-improvements in biomanufacturing could therefore be achieved by arbitraging how cells receive cellular energy. The O₂-tolerant hydrogenase from C. necator has been adapted into a versatile, modular, and modifiable tool for industrial microbiology. Furthermore, it is demonstrated using two NADH donors (H₂ and formate) that external reducing power obviates the decarboxylative oxidation of feedstock, with up to perfect efficiency. The retained feedstock can then be stoichiometrically converted into additional product-of-interest. In theory, this core principle is applicable regardless of carbon source, media composition, electron donor, or target molecule. This process and technology therefore potentiate an exceptionally broad means of decarbonizing biomanufacturing while simultaneously enhancing its cost-competitiveness, both of which will be vital for safeguarding energy security, preserving ecosystems, and advancing global socioeconomic development.

In this work, it is demonstrated how either H₂ or formate can be fed to an industrial microorganism to prevent the decarboxylative metabolism of feedstock, in turn, resulting in enhanced production of a commodity product.

### Materials and Methods

Genetic manipulation: Routine PCR was carried out using Phusion polymerase hot start II, and cloning using NEB HiFi DNA assembly. PCR reactions were carried out with Phusion Hot Start II polymerase or Q5 Hot start polymerase and were carried out according to the manufacturer's instructions. The thermocycler (Applied Biosystems) was programmed as follows: 1 min at 94° C.; 25 rounds of 10 sec at 94° C.|15 sec at Tₘ−4° C.| 20 sec/kb at 72° C.; 20 sec/kb at 72° C. The amplicons were resolved by agarose electrophoresis and were evaluated by a gel imager (Bio-Techne). PCR fragments were then purified using the QIAquick gel extraction kit (Qiagen). Plasmid assembly was achieved using the NEBuilder HiFi DNA Assembly Master Mix (New England BioLabs), or using T4 DNA Ligase (ThermoFisher). Candidate plasmids were screened by transformation into chemically competent “Stellar” E. coli (Takaro Bio). Authenticated plasmids were transformed by electroporation technique into triple-washed DE3-competent BW25113 E. coli. Candidate transformants were evaluated using a similar culturing, plasmid extraction, and restriction enzyme digestion scheme. Routine Sanger sequencing was carried out using Azenta's services, and whole plasmid sequencing was performed when necessary (Primordium Labs).

Media: Three culturing medias were used: (1) Miller LB media (Millipore); (2) EZ Rich Defined Medium Kit (Teknova), with one of the following primary carbon sources: (i) 0.02775M glucose (ii) 0.02775M xylose (iii) 0.02775M potassium gluconate, (iv) 0.0555M sodium pyruvate (v) 0.0555M sodium acetate (vi) 0.02775M disodium succinate hexahydrate (vii) 0.02775M disodium malate; (3) M9 minimal media containing (per 1 L) 11.28 g 5×M9 minimal salts, 0.12 g MgSO₄, 0.011 g CaCl₂), and 5 g glucose (Sigma). The LB media was sterilized by autoclaving and the defined medias were sterilized by filtration through 0.2 μm filters.

Growth on H₂ gas and evaluation: Glycerol stocks of BW25113 E. coli transformants were plated on LB agar with appropriate antibiotics (Teknova). Colonies were seeded into the appropriate media and grown aerobically overnight (<18 h) at 37° C. and 150 RPM. The experimental culture was established by inoculating 200 μL of seed culture into 10 ml of the same media used to create the seed culture. Incubations were performed in glass serum vials with 112 mL headspace. Cultures were initially grown aerobically at 37° C. and 150 RPM for 4 h. Cells were then moved to a 30° C. warm room and were induced for relevant gene expression. The vials where then closed with a butyl stopper and were crimped to make the flasks air tight. Then, 20 mL of the atmospheric air in the headspace was removed using a syringe, and 20 mL of H₂ gas (experimental cultures) or 20 mL of N₂ gas (control cultures) was added. Cells were then grown for 18 h at 150 RPM and 30° C. Gas Pressure Sensor (Vernier) monitored internal gas pressure.

Analysis of H₂, O₂, N₂, and CO₂ content by gas chromatography: Gas analysis was performed using a Shimadzu GC-2014 gas chromatograph. Aliquots of gas equivalent to 10 ml at standard temperature and pressure (STP) were withdrawn from each plugged serum bottle via a luer-lock syringe fitted with a stopcock valve and sterile 21 G needle, and individually injected into the GC. Samples flowed isothermally through a 2 mm×8 m Shimadzu column (60° C.) using argon carrier gas. The H₂, O₂, and N₂ were quantified by 8.5 min of flow-time through a TCD module (120° C.), and CO₂ was quantified by an additional 4.0 min of flow-time through the methanizer (380° C.) and FID module (200° C.). The gas profile was quantified by interpolating peak integrations to standard plots, using N₂ as an internal standard.

Metabolite quantification with HPLC: The extracellular fermentation profile was evaluated using HPLC. Samples were prepared by cooling 1 ml of broth aliquots in ice water. Samples were centrifuged, and the supernatant was retained. The supernatant was mixed in a 9:1 ratio with methanol to quench reactions and provide an internal HPLC retention standard. Samples were immediately filtered through 0.45 μm modified nylon column (VWR). The filtrate was stored at −80° C. until needed. Filtrate was analyzed isothermally using an Aminex HPX-87H column (BioRad) set to 65° C. and a refractive index detector set to 50° C. (Agilent). The autoloading sample tray was set to 4° C. The mobile phase was 0.005M H₂SO₄ and flowed at 0.6 ml/min. Data acquisition and analysis were performed using ChemStation software (Agilent). The fermentation profile was quantified by interpolating peak integrations to standard plots, using methanol as an internal standard.

Quantifying metabolites using LC-MS: Organic acids were measured via reversed phase chromatography and high-resolution mass spectrometry. Liquid chromatography (LC) was conducted on an Ascentis Express RP-Amide column (150-mm length, 4.6-mm internal diameter, and 2.7-μm particle size (Sigma-Aldrich), equipped with the appropriate guard column, using an Agilent Technologies 1260 Series high performance liquid chromatography (HPLC) system (Agilent). A sample injection volume of 2 μL was used throughout. The sample tray and column compartment were set to 5 and 50° C., respectively. The mobile phase solvents used in this study were of LC-MS grade (HONEYWELL™, charlotte, NC). Other chemicals and reagents were purchased from Sigma-Aldrich. The mobile phases were composed of 0.1% formic acid/84.9% water/15% methanol (v/v/v) (A) and 0.04% formic acid and 5 mM ammonium acetate in methanol (B). Metabolites were separated via gradient elution under the following conditions: Linearly increased from 0% B to 30% B in 5.5 min, increased to 100% B in 0.2 min, held at 100% B for 2 min, decreased from 100% B to 0% B in 0.2 min, and held at 0% B for 2.5 min. The flow rate was held at 0.4 mL/min for 7.7 min, linearly increased from 0.4 mL/min to 1 mL/min in 0.2 min, and held at 1 mL/min for 2.5 min. The total LC run time was 10.4 min. The HPLC system was coupled to an Agilent Technologies 6545 series quadrupole time-of-flight mass spectrometer (QTOF-MS). MS conditions were as follows: Drying and nebulizing gases were set to 10 L/min and 25 lb/in2, respectively, and a drying-gas temperature of 300° C. was used throughout. Sheath gas temperature and flow rate were 330° C. and 12 L/min, respectively. Electrospray ionization, via the Agilent Technologies Jet Stream Source, was conducted in the negative ion mode and a capillary voltage of 3,500 V was utilized. The fragmentor, skimmer, and OCT 1 RF Vpp voltages were set to 100, 50, and 300 V, respectively. The acquisition range was from 70-1,100 m/z, and the acquisition rate was 1 spectra/sec. Prior to data acquisition, the QTOF-MS system was tuned with the Agilent ESI-L Low concentration tuning mix (diluted 10-fold in a solvent mixture of 80% acetonitrile and 20% water) in the range of 50-1700 m/z. Reference lock masses from Data acquisition and processing were conducted via the Agilent MassHunter software package. Reference mass correction was performed with 5 μM trifluoroacetic acid ammonium salt (part number 18720243) and 5 μM HP-0921 (part number 18720241) at a flow rate of 5 μL/min via a second ESI sprayer. Data processing and analysis were conducted via Agilent MassHunter Qualitative Analysis, Agilent Profinder, and/or Agilent MassHunter Quantitative analysis. All other metabolites were analyzed according to the method described by Amer et al. (2022). Metabolites were quantified via seven-point calibration curves from 0.39 to 25 μM.

Proteomics analysis: Following typical cultivation of bacterial cultures, 5 ml of broth was centrifuged, and the pellet was retained. The cells were twice washed with water, harvested and stored at −80° C. until further processing. Protein was extracted from cell pellets and tryptic peptides were prepared by following established proteomic sample preparation protocol (Chen et al. 2023). Briefly, cell pellets were resuspended in Qiagen P2 Lysis Buffer (Qiagen) to promote cell lysis. Proteins were precipitated with addition of 1 mM NaCl and 4× vol acetone, followed by two additional washes with 80% acetone in water. The recovered protein pellet was homogenized by pipetting mixing with 100 mM ammonium bicarbonate in 20% methanol. Protein concentration was determined by the DC protein assay (BioRad). Protein reduction was accomplished using 5 mM tris 2-(carboxyethyl)phosphine (TCEP) for 30 min at room temperature, and alkylation was performed with 10 mM iodoacetamide (IAM; final concentration) for 30 min at room temperature in the dark. Overnight digestion with trypsin was accomplished with a 1:50 trypsin:total protein ratio. The resulting peptide samples were analyzed on an Agilent 1290 UHPLC system coupled to a Thermo Scientific Orbitrap Exploris 480 mass spectrometer for discovery proteomics (Chen et al. 2022). Briefly, peptide samples were loaded onto an Ascentis® ES-C18 Column (Sigma-Aldrich) and were eluted from the column by using a 10 minute gradient from 98% solvent A (0.1% FA in H₂O) and 2% solvent B (0.1% FA in ACN) to 65% solvent A and 35% solvent B. Eluting peptides were introduced to the mass spectrometer operating in positive-ion mode and were measured in data-independent acquisition (DIA) mode with a duty cycle of 3 survey scans from m/z 380 to m/z 985 and 45 MS2 scans with precursor isolation width of 13.5 m/z to cover the mass range. DIA raw data files were analyzed by an integrated software suite DIA-NN. The database used in the DIA-NN search (library-free mode) is E. coli latest Uniprot proteome FASTA sequences plus the protein sequences of the heterologous proteins and common proteomic contaminants. DIA-NN determines mass tolerances automatically based on first pass analysis of the samples with automated determination of optimal mass accuracies. The retention time extraction window was determined individually for all MS runs analyzed via the automated optimization procedure implemented in DIA-NN. Protein inference was enabled, and the quantification strategy was set to Robust LC=High Accuracy. Output main DIA-NN reports were filtered with a global FDR=0.01 on both the precursor level and protein group level. The Top3 method, which is the average MS signal response of the three most intense tryptic peptides of each identified protein, was used to plot the quantity of the targeted proteins in the samples (Ahrné al. 2013; Silva et al. 2006).

Recombinase-Assisted Genome Engineering (RAGE): RAGE plasmids were constructed using yeast recombinational cloning. Briefly, PCR fragments (˜5 kb each) and ˜300 bp gblock linkers containing homology to the PCR fragments for assembly into plasmid pW5Y were transformed into yeast strain CEN.PK using the Frozen-EZ Yeast Transformation II Kit (Zymo Research) and colonies were recovered on CSM-Uracil plates. Plasmids were purified from yeast using the Zymoprep Yeast Plasmid Miniprep II (Zymo Research) and sequenced in collaboration with Agile BioFoundry and DIVA sequencing. Correct plasmids were introduced into bacteria by electroporation using SIG-10 ultracompetent cells (Sigma-Aldrich). A chloramphenicol cassette flanked by loxP and lox5171 sites was introduced to strains using lambda red recombineering targeting the flik locus. RAGE vectors encoding Cre recombinase were then electroporated into the target strain and were screened for Apramycin resistance and Chloramphenicol sensitivity, which indicated Cre-mediated recombination of RAGE cassette into the flik locus.

Measurement of activity in H2-ase and FDH: Cells were inoculated from an overnight pre-culture in LB media with appropriate antibiotics and were induced for hydrogenase expression at 30° C. in the mid-log phase. Cells were grown for 24 hr, were pelleted on ice and were lysed using sonication. Bradford assays were performed to quantify proteins. Measurement of hydrogenase activity was performed as previously described, with modifications. The assay was modified by performing kinetic assays in an anaerobic chamber, in 4% H₂ and 10% CO₂, balanced with N₂ on a plate reader.

### Results and Discussion

### 1. Creating a Portable and Versatile Hydrogenase Platform

1.1 Adoption of C. necator Hydrogenase

The electrons carried by H₂ can enter cells by passive diffusion and then be incorporated into cellular metabolism by reducing nicotinamide (NAD⁺) with hydride equivalent (H⁻). This is performed by hydrogenases, which are composed of HoxFUHY heterotetramers (Lubitz et al. 2014). The HoxHY module contains a [NiFe] catalytic core and is responsible for the heterolytic cleavage of H₂ into protons and hydride equivalents (Lauterbach et al. 2011a). The HoxFU module recruits NAD⁺ and reduces it to NADH using an aqueous proton along with electrons passed from the HoxYH didomain through [4Fe-4S] clusters (Lauterbach et al. 2011b).

Until now, technical issues have prohibited the use of hydrogenases as a generalizable tool for industrial microbiology. In addition to the heterotetramer, nine maturation proteins are also required to install homo- or heterometallic active sites, iron-sulfur clusters, and perform other post-translational modifications on the hydrogenase. Thus, 13 soluble proteins must be simultaneously expressed within the host organism to harness H₂ as reducing power (English et al. 2009; Fan et al. 2020). Hydrogenases are sensitive to O₂ and are irreversibly inactivated upon contact, restricting their use to anaerobic fermentation (Shafaat et al. 2013; Lu & Koo, 2019). A key exception is the hydrogenase within Cupriavidus necator (formerly Ralstonia eutropha) (Lauterbach & Lenz, 2013). This homolithoautotroph has drawn great attention for its ability to use H₂ to fixate CO₂ in aerobic environments to create bioplastics and biofuels (Panich et al. 2021; Zhang et al. 2022). Aerobic tolerance is enabled by an unusual [4Fe-3S] electron transfer relay that allows the hydrogenase to expend electrons to dislodge oxygen from the heterometallic core by reducing it to water (Parkin & Sargent, 2012; Pandelia et al., 2012).

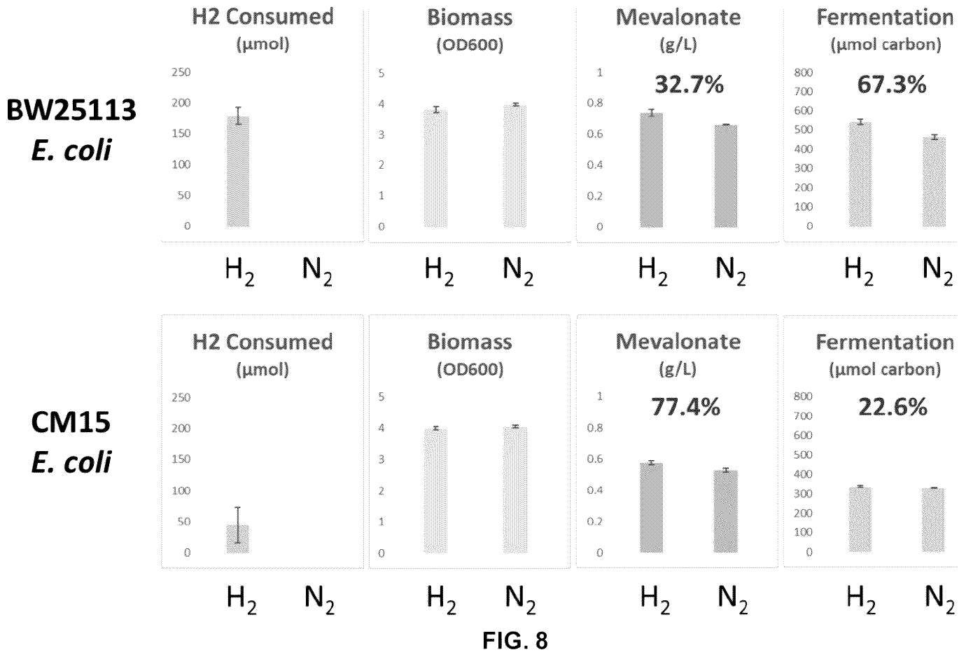

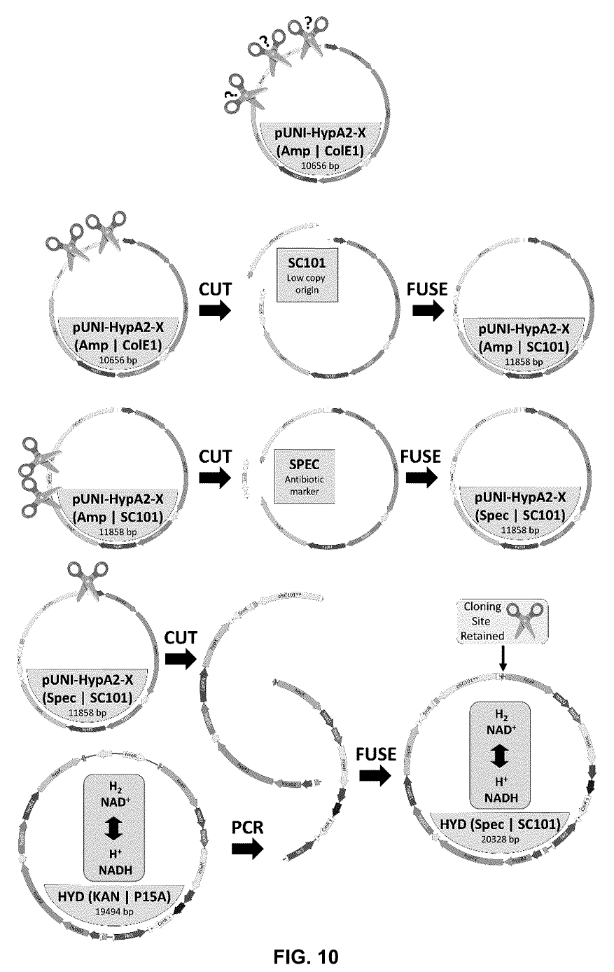

C. necator hydrogenase was chosen. Its functional expression is already reported in E. coli and P. putida, providing us with a catalog of genetic components and protocol steps. However, existing configurations were non-ideal for various reasons such as missing genes, non-codon-optimized genes, multiple plasmids, or plasmids intended for protein purification (Ghosh et al. 2013; Schiffels et al. 2013; Lonsdale et al. 2015; Lamont & Sargent, 2017; Teramoto et al. 2022; Fan et al. 2022). Parts kindly donated by Lamont and Sargent (2017) were adopted and all 13 genes were consolidated into a single plasmid under medium copy P15A origin of replication and kanamycin selection (FIG. 9). The prototype contains two operons, the first encoding the heterotetramer and select maturation genes and are inducible by T5 polymerase (IPTG), and the second encoding the remaining maturation genes and are constitutively expressed. In addition, the entire 13-gene cassette was chromosomally integrated into BW25113 E. coli via the flik locus to create a plasmid-free expression strain. The function of both plasmid and integrated hydrogenase prototypes were tested using a devised gas cultivation protocol (FIG. 11). Briefly, E. coli transformants were cultivated in bottles that can be plugged with rubber stoppers, enabling the injection of controlled and quantifiable amounts of pure gases into the sealed cultures. Changes in gas composition and attendant effects upon bacterial metabolism are then evaluated using GC and HPLC techniques.

### 1.2 Function Tests of Prototype Plasmid

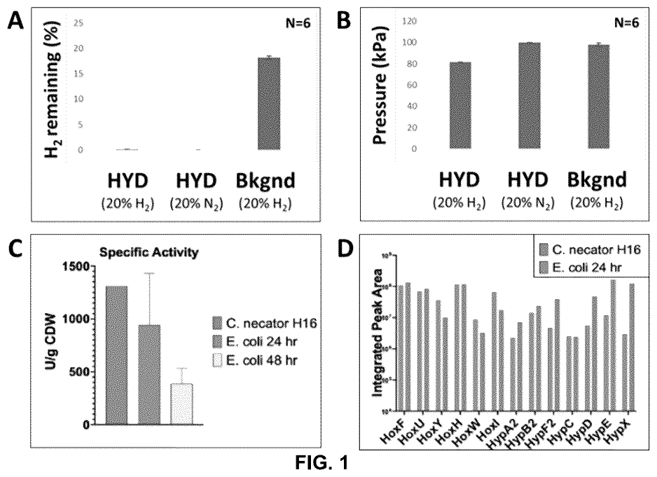

It was first determined whether these constructs were functional. It was observed that when BW251113 E. coli containing hydrogenase plasmid is fed with 20% H₂ in air, almost all the H₂ is consumed after an overnight cultivation in LB media. In contrast, E. coli lacking hydrogenase plasmid exhibited no H₂ consumption, thereby providing initial evidence that the prototype was functional (FIG. 1A). Moreover, the consumption of H₂ produces measurable declines in internal bottle pressure, such that pressure readers can be used to rapidly and qualitatively evaluate the outcome of experiments (FIG. 1B). Hydrogenase activity within the integrated variant was also assayed, but was observed to underperform as compared to the plasmid version, perhaps indicating that gene copy number is important to achieve a robust phenotype. It was pivoted to the plasmid prototype for remaining experiments.

Though H₂ depletion was successfully observed, there was concern that the exogenous hydrogenase could be partially impaired for anomalous reasons (Rosano & Ceccarelli, 2014). Therefore, it was next performed a biochemical assay to compare the specific activity of C. necator hydrogenase when expressed in E. coli as compared to activity within the native organism. Following cultivation of both organisms for 24 h, lysis, and monitoring for NADH formation (Lupacchini et al. 2021), it was observed that the specific activity of the C. necator hydrogenase expressed in E. coli (942+/−489 U/g CDW) was commensurate with activity observed in C. necator lysates (1310 U/g CDW) (FIG. 1C).

To mitigate the energy burden caused by expressing 13 proteins, the optimal production of the hydrogenase would require each member of the heterotetramer (HoxFUHY) to be expressed at similar levels and that maturation proteins would be produced at a minimally required level to effectively maturate the hydrogenase. As bacteria have evolved to optimize protein production to satisfy fitness demands, it could reasonably be expected that heterologous expression could impair this balance (Bosdriesz et al. 2015; Molenaar et al. 2009; Vogel & Marcotte, 2012). To evaluate the degree of impairment, quantitative proteomics on each of the 13 heterologous proteins expressed in E. coli was performed and compared these results to autotrophically grown C. necator (Batth et al. 2012; Alonso-Gutierrez et al. 2015; Petzold et al. 2015). It was observed that production of HoxFUHY in was more closely balanced in E. coli than in the native organism (FIG. 1D). However, many maturation proteins are expressed at a higher level in C. necator, in some cases, one order of magnitude greater. This may suggest that the plasmid is under-servicing the heterotetramer, perhaps because of our choice to place many maturation proteins under constitutive expression rather than an intensely inducible promoter. However, this outcome is difficult to interpret as C. necator is also known for an unusually wasteful protein expression profile (Jahn et al. 2021). Overall, it is concluded that the heterologous hydrogenase platform does not impose an exorbitantly wasteful burden upon cellular energy resources, as compared to hydrogenase when produced in the native organism.

### 1.3 Establishing Mature Hydrogenase Library and Platform

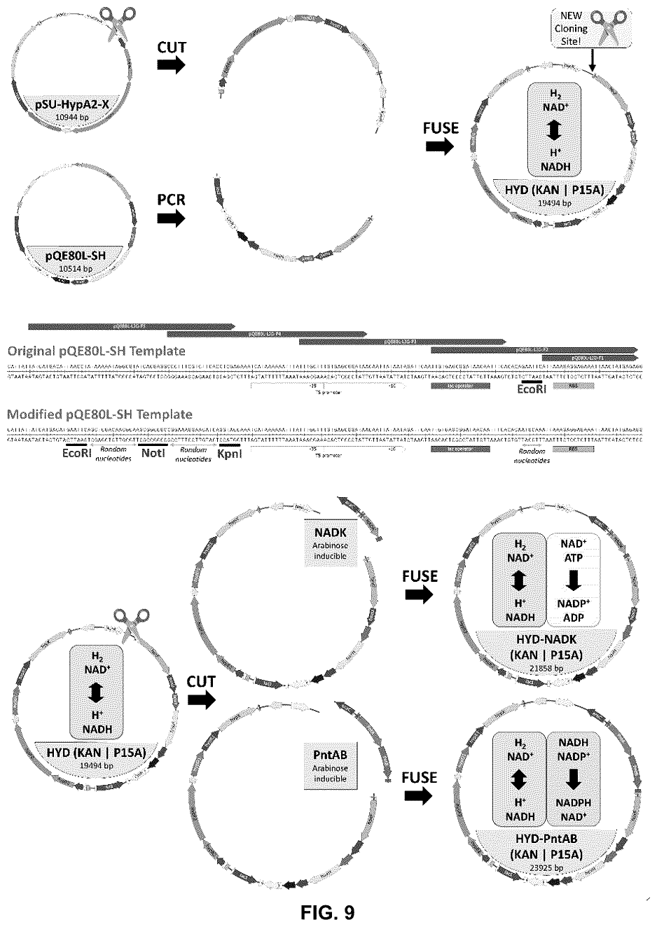

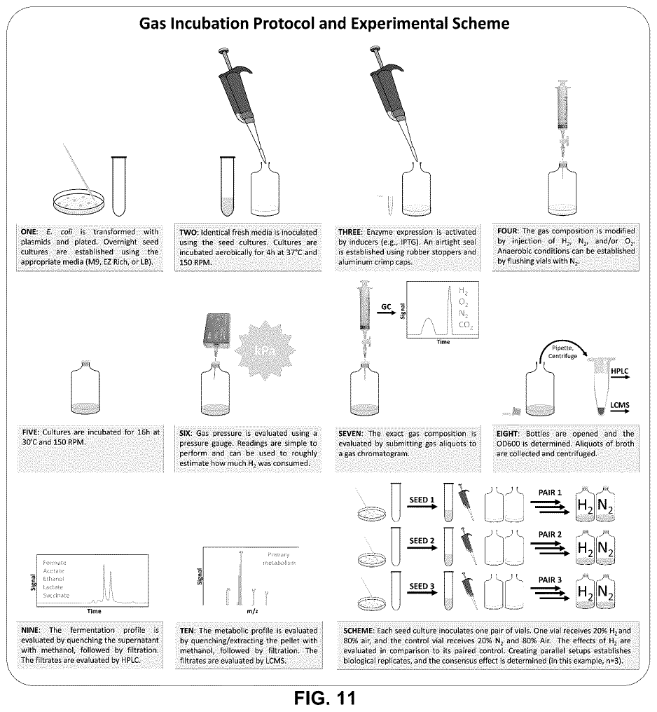

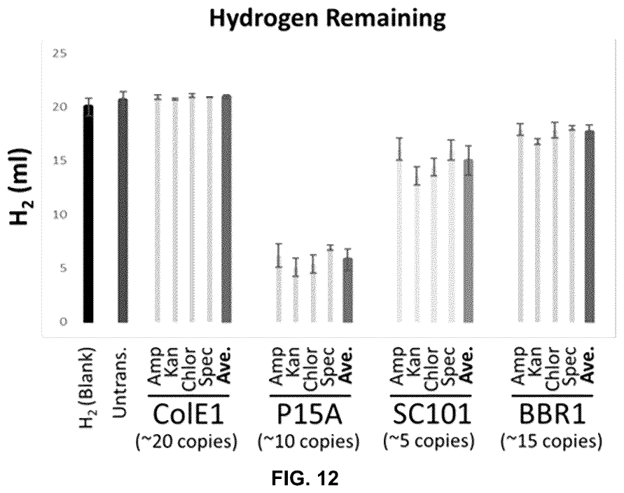

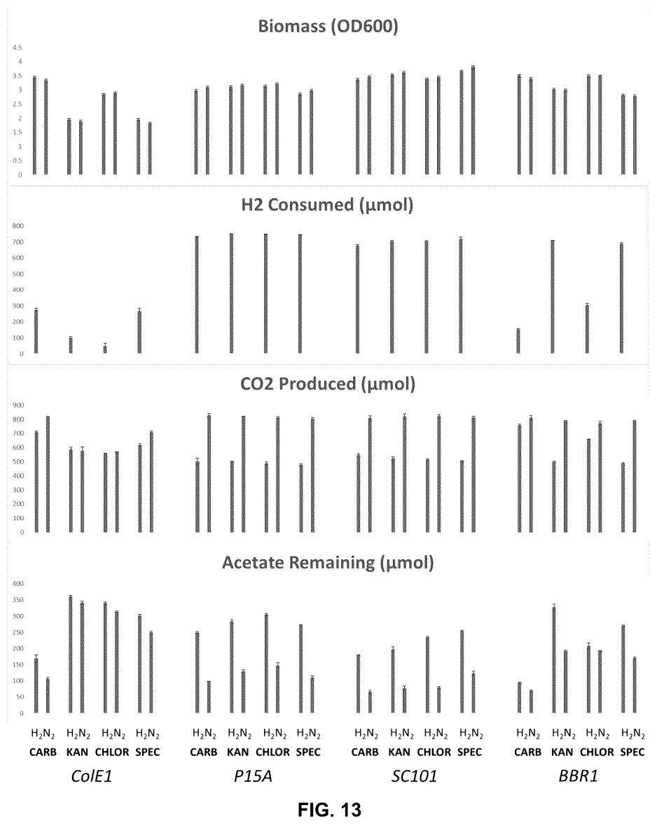

The widespread adoption of H₂ feeding in industrial microbiology will require the creation of versatile and portable genetic hydrogenase components. However, in general, the absence of standardization, non-modularity, and incompatible genetic components plague synthetic biology (Kwok, 2010). Inspired by the BioBrick plasmid library and analogous works within our lab (Shetty et al. 2008; Lee et al. 2011), the prototype hydrogenase plasmid was improved upon by developing a standardized, versatile, modular, and modifiable library of hydrogenases, each of which can be immediately added to a synthetic biology project or reassembled to meet custom needs (FIG. 10). Sixteen plasmids, each containing the full complement of 13 genes, are available in four origins (ColE1, P15A, SC101, BBR1) and four selection markers (carbenicillin, kanamycin, chloramphenicol, spectinomycin) (FIG. 1E). Each member of this library was assayed for H₂ consumption within BW25113 E. coli cultivated in undefined and defined medias (FIGS. 12 and 13). An additional feature is the inclusion of Kpn-NotI cloning site for the sub-customization of any member of this library. For example, two constructs of HYD (Kan:P15A), encoding hydride exchange proteins NADK or PntAB under an arabinose-inducible promoter, are available to facilitate the robust regeneration of NADPH directly from H₂, as evidenced by simultaneous H₂ consumption assays as well as NADH- and NADPH-specific colorimetric assays (FIG. 14). This library plus all plasmids described in this work are available for requisition via our institution's ICE registry (Ham et al. 2012) using the catalog numbers provided in Table 2.

TABLE 2
Plasmids used in this Example 1.

| | | Size |
|---|---|---|
| Name | Description | (kb) |
| UNI (Carb: ColE1) | Sub-components used to assemble | 10.7 |
| UNI (Kan: ColE1) | library of hydrogenase plasmids | 11.0 |
| UNI (Chlor: ColE1) | (see FIG. 11). | 10.5 |
| UNI (Spec: ColE1) | Additional customization is | 10.6 |
| UNI (Carb: P15A) | enabled by SalI, AatII, and EcoRI | 10.5 |
| UNI (Kan: P15A) | cut-sites that flank each origin of | 10.9 |
| UNI (Chlor: P15A) | replication and antibiotic | 10.4 |
| UNI (Spec: P15A) | resistance marker. | 10.5 |
| UNI (Carb: SC101) | Encodes HypA2, HypB2, HypF2, | 11.9 |
| UNI (Kan: SC101) | HypC1, HypD1, HypE1, HypX. | 12.2 |
| UNI (Chlor: SC101) | Expression is achieved via | 11.7 |
| UNI (Spec: SC101) | constitutive TATA promoter. | 11.8 |
| UNI (Carb: BBR1) | | 10.9 |
| UNI (Kan: BBR1) | | 11.3 |
| UNI (Chlor: BBR1) | | 10.8 |
| UNI (Spec: BBR1) | | 10.9 |
| HYD (Carb: ColE1) | Complete hydrogenase library (see | 19.2 |
| HYD (Kan: ColE1) | FIG. 1 and FIG. 11). | 19.5 |
| HYD (Chlor: ColE1) | Contains NotI and KpnI cloning | 19.0 |
| HYD (Spec: ColE1) | site, but neither promoter nor | 19.1 |
| HYD (Carb: P15A) | terminator are provided. | 19.1 |
| HYD (Kan: P15A) | Encodes | 19.4 |
| HYD (Chlor: P15A) | HypA2, B2, F2, C1, D1, E1, X, via | 18.9 |
| HYD (Spec: P15A) | constitutive TATA promoter, and | 19.0 |
| HYD (Carb: SC101) | HoxF, U, Y, H, W, I via T5 (IPTG). | 20.4 |
| HYD (Kan: SC101) | Cloning artifacts include psuedo- | 20.8 |
| HYD (Chlor: SC101) | chloramphenicol marker (non- | 20.2 |
| HYD (Spec: SC101) | functional). | 20.3 |
| HYD (Carb: BBR1) | | 19.5 |
| HYD (Kan: BBR1) | | 19.8 |
| HYD (Chlor: BBR1) | | 19.3 |
| HYD (Spec: BBR1) | | 19.4 |
| HYD (Carb: P15A)- | Combined T5 (IPTG) hydrogenase | 20.7 |
| FDH-1 | & TATA (constitutive) Formate | |
| | Dehydrogenase | |
| HYD (Kan: P15A)- | Combined T5 (IPTG) hydrogenase | 23.9 |
| PntAB | & AraBAD (arabinose) PnTAB. | |
| HYD (Kan: P15A)- | Combined T5 (IPTG) hydrogenase | 21.9 |
| NADK | & AraBAD (arabinose) NAD | |
| | Kinase. | |
| HYD (Kan: P15A)-MEV | Combined T5 (IPTG) hydrogenase | 23.6 |
| | & T7 (IPTG) mevalonate pathway | |
| FDH (Spec: ColE1) | Formate dehydrogenase with | 3.3 |
| | constitutive TATA promoter | |
| FDH (Spec: P15A) | Formate dehydrogenase with | 3.3 |
| | constitutive TATA promoter | |
| RppA-1 (Chlor: BBR1) | Flaviolin-producing enzyme with | 7.2 |
| | AraBAD (arabinose) induction | |
| RppA-2 (Chlor: BBR1) | Flaviolin-producing enzyme with | 5.0 |
| | Trc (IPTG) induction | |
| MEV (Chlor: ColE1) | Produces mevalonate; encodes | 7.9 |
| MEV (Chlor: P15A) | acetoacetyl-CoA thiolase, HMG- | 7.5 |
| MEV (Chlor: SC101) | CoA synthase, and HMG-CoA | 9.0 |
| | reductase; T5 (IPTG) promoter | |

### 2. Supplementing Reducing Power for Generalizable Yield Improvements in Industrial Microbiology

### 2.1 Principles and General Methodological Approach

The fundamental principle of this work is that cofactor feeding (i.e., feeding an industrial microorganism with H₂, formate, or other source of electrons) enhances the yield of a molecule of interest. This is because the feedstock no longer needs to be oxidized to create bioenergy. For implications arise:
(1) Cofactor feeding ought to improve the yield of bioproducts, including those that do not use NAD(P)H as a reductant within their biosynthetic pathways. We will demonstrate this effect using the non-reduced polyketide flaviolin, produced by the type III polyketide synthase RppA. Due to its simplicity of formation and its red pigmentation, it is used as a malonyl-CoA biosensor (Yang et al. 2018)'
(2) Various NADH donors ought to have comparable effects on host metabolism and product yield. This will be examined using formate dehydrogenase, which can regenerate NADH (or NADPH) by oxidizing formate to CO₂. As these enzymes are already widely developed for biotechnological applications, the FDH from Pseudomonas sp. 101 was chosen for simplicity (Wenk et al. 2020).
(3) This principle can be realized using multiple carbohydrate feedstocks or media compositions. The experiments were varied to include undefined LB, minimal media, and defined rich media, either containing high energy-dense feedstocks such as glucose and low energy-dense feedstocks such as acetate.
(4) This principle can be realized to improve the yield of multiple classes of bioproducts. Flaviolin could be considered an archetypal member of the polyketide class of natural products. To demonstrate applicability beyond polyketides, the same phenomenon be demonstrated using mevalonate, the key metabolic intermediate leading to the isoprenoids.

### 2.2 Enhancing Flaviolin Production by NADH Donors H₂ and Formate

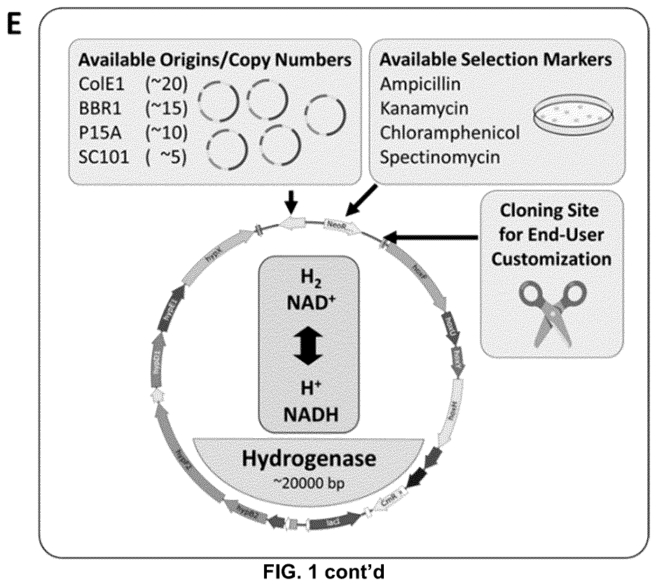

Applying cofactor feeding to increase the yield of molecules that do not need NADH as a reductant is not an apparent application of this technology and method. The formation of non-reduced polyketide flaviolin is exclusively dependent upon malonyl-CoA availability (Yang et al. 2018). Therefore, increasing malonyl-CoA supply can only be achieved by increasing the feedstock available to become malonyl-CoA. E. coli transformed with hydrogenase and flaviolin-producing RppA were grown in undefined LB media. These cultures were incubated in either 20% H₂ in air or a control treatment of 20% N₂ in air. Hydrogen gas increased flaviolin yield by 75% (FIG. 2), providing prima facie evidence of the feedstock retention principle.

### 2.3 Evaluating Bioenergetic Efficiency of Feedstock Retention by H₂ and Formate

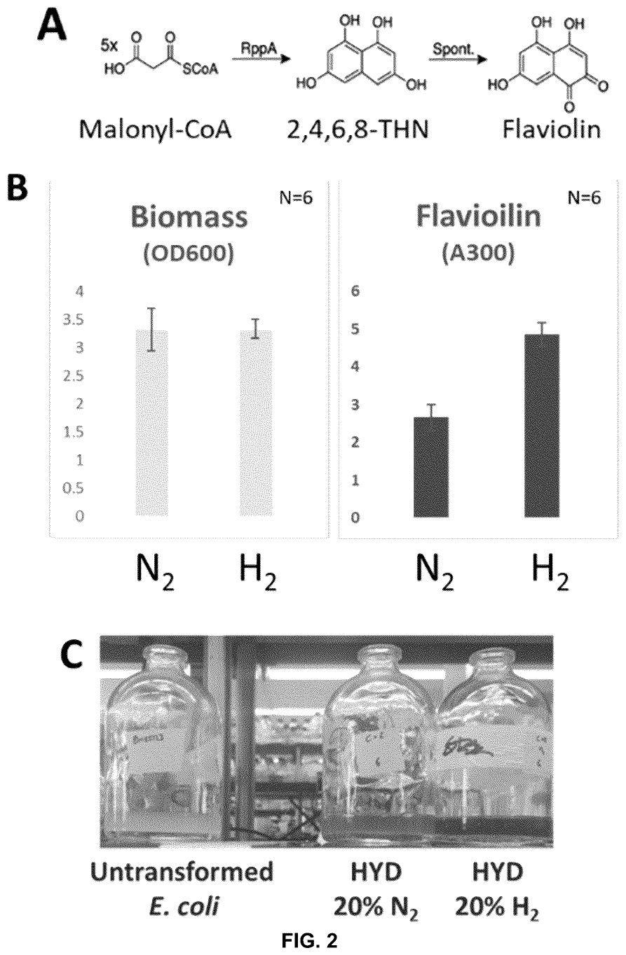

Bioenergy takes various forms. To unite NADH, NADPH, FADH₂, and ATP by a common quantifiable term, these energy carriers are expressed in terms of proton motive force (PMF) equivalencies (FIG. 3). In the preceding section, qualitative evidence that cofactor feeding spares feedstock from oxidative decarboxylation is provided. To evaluate this relationship quantitatively, acetate was used as a feedstock because its metabolism is simple and well-understood. Acetate is actively imported at the expense of a proton by the H/acetate symporter acetate permease (Orr et al. 2019). The acetate must then be converted into acetyl-CoA by acetyl-CoA synthetase by investing two ATP. This carbon may then be fully digested to CO₂, yielding 3 NADH, 1 FADH₂, and 1 GTP/ATP. In summary, energy equivalent to 9 H⁺ PMF was invested, and 40 H⁺ PMF reaped, netting 31 H⁺ PMF.

Each mole of H₂ or formate can be oxidized to regenerate NADH (10 H⁺ PMF) by their respective enzymes with >99% efficiency in ambient air (Lauterbach & Lenz, 2013). Thus, one must provide 3.1 moles of H₂ to prevent the oxidation of one mole of acetate. To determine the required number of moles of H₂ experimentally, these cofactors can be titrated on E. coli cultivated on acetate. The results of these titrations can be compared to theory to determine the efficiency of these electron donors at preventing decarboxylative metabolism. This identical work can then be performed on formate. Although formate can be oxidized to regenerate NADH stoichiometrically, the transportation of formate across cell membranes occurs as (neutrally charged) formic acid. Therefore, each formate also contributes a single proton towards electromotive potential. For that reason, only 2.82 moles of formate are theoretically needed to prevent the oxidation of one mole of acetate.

To test bioenergy efficiency, seed cultures of E. coli bearing either hydrogenase were inoculated into rich defined media (EZ Rich-Takara) with 55.5 mM acetate as primary carbon source, and grown in open bottles undisturbed for 4 h at 37° C. The bottles were then plugged, and the cells were fed various amounts of H₂ or formate before a second period of undisturbed incubation lasting 16 h at 30° C. Consumption of H₂, and acetate, and the production of CO₂ and biomass, were all compared to these values at the onset of NADH feeding (4 h).

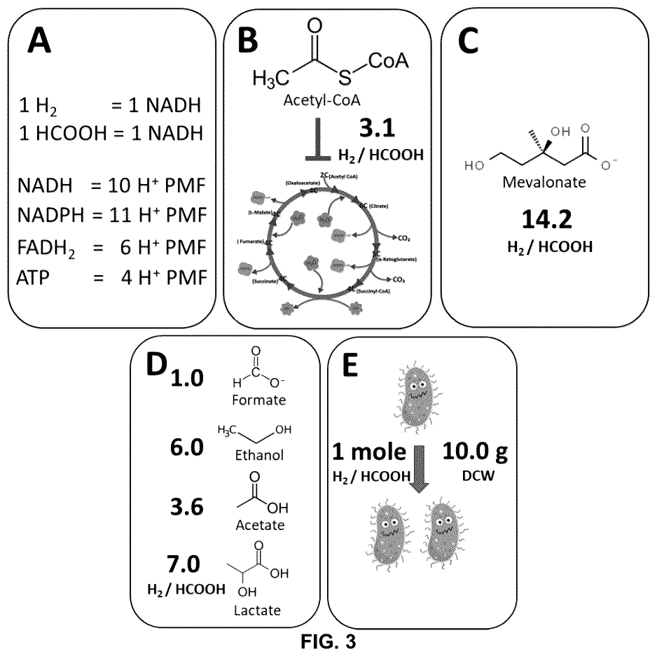

Results show that E. coli consumed nearly all of the 0 to 20 ml of H₂ provided (equal to 0 to 800 μmol) (FIG. 4). This consumption had zero apparent effect on total biomass formation, evidenced by statistically identical OD600 values. The presence of equal biomass means that total biochemical work to be achieved within this titration is identical. Importantly, there is a robust titratable relationship between H₂ consumption and acetate preservation. This reveals that H₂ allows E. coli to form the identical amount of biomass while requiring less acetate to do so. Moreover, a titratable decline in CO₂ demonstrates that H₂ is preserving acetate by preventing its decarboxylative oxidation to cellular energy. From these data, the moles of H₂ required to prevent such oxidation was determined to be 3.925. As compared to the theoretical minimum ratio of 3.1, this provides H₂ with 79% bioenergy efficiency. The number of moles of H₂ that are needed to prevent the formation of CO₂ can be similarly determined, and correlating these results with moles of acetate retained provides the predicted stoichiometry of 2, within error (FIG. 4).

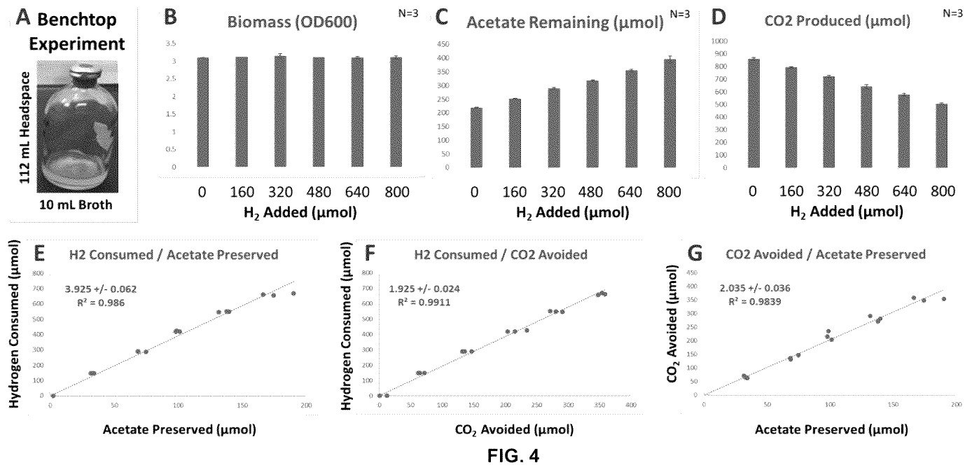

The identical titration was then performed using formate dehydrogenase by feeding E. coli between 0 to 400 μmol of sodium formate (FIG. 5). As before, total biomass formation was identical, and a titratable relationship with acetate emerged. As CO₂ is the side-product of the oxidation of format, CO₂ content within serum bottles increased with increasing formate load. Subtracting moles of formate consumed from total CO₂ content therefore provides the amount of CO₂ arising from all other metabolism. Cross-referencing these values with moles of acetate preserved reveals that both values obey stoichiometry. The number of moles of formate that was experimentally determined to prevent the consumption of acetate was 2.865+/−0.102. This is within the margin of error of the theoretical minimum of 2.82, indicating that formate is bioenergetically perfect (FIG. 5). These experiments reveal that multiple electron donors can be used to prevent the decarboxylative metabolism of substrate with great efficiency.

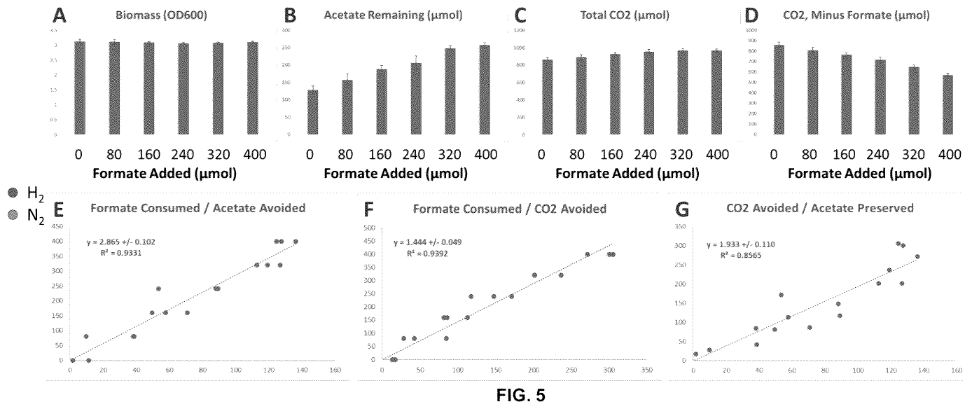

Depending on metabolic context, acetate can either serve as a substrate for consumption or a product of fermentation (Wolfe, 2005). To disentangle this alternative interpretation of the aforementioned data, an additional experiment was performed. H₂, CO₂, and acetate were evaluated by time-course experiment. If an ‘acetate switch’ was occurring, the concentration of broth acetate would be expected to increase in response to H₂ over time, before eventually declining as E. coli starts eating it, producing a “peak and trough” pattern. By preparing technical replicates of E. coli seed cultures inoculated into the identical rich acetate media, and harvesting these cultures after a predetermined period of incubation, one can track the evolution of H₂, CO₂, and acetate trends over time. This experiment revealed that no such “peak and trough” pattern emerged (FIG. 6). Changes in CO₂ evolution, acetate concentration, and H₂ consumption began occurring concomitantly at 8 h post-inoculation. As H₂ feeding began at 4 h post-inoculation, this reveals that E. coli requires two to four hours to translate and fold hydrogenase proteins. Peak H₂ consumption was observed at 5.4 mmol H₂/g_{biomass}/h, and adopted a three-phase sigmoidal profile, perhaps indicating periods of enzyme assembly, peak energy demand, and non-peak demand In conclusion, robust and quantitative biochemical evidence that both H₂ and formate can efficiently prevent the decarboxylative oxidation of feedstock are provided.

### 2.4 Quantifying Yield Enhancement Efficiency by H₂ and Formate

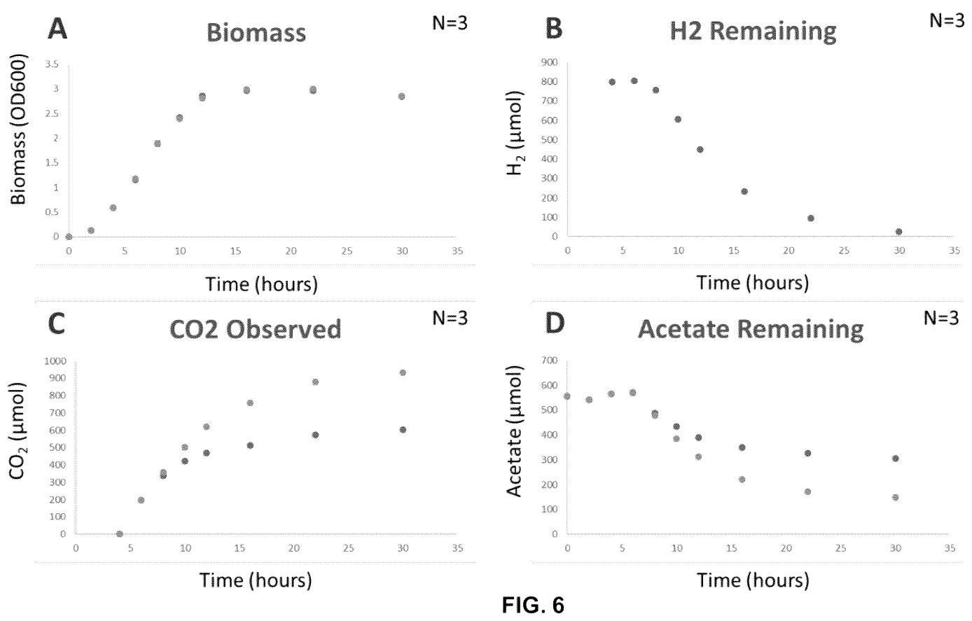

Consider the example of cultivating E. coli on glucose as the sole carbon source to produce mevalonate, the key metabolic intermediate leading to terpene monomers isopentenyl pyrophosphate and dimethylallyl pyrophosphate. Upon cofactor feeding, the host may use the supplied bioenergy to achieve up to three possible outcomes relative to an untreated control: (1) Increase mevalonate biosynthesis; (2) Dispose of the additional reducing power by increasing fermentation, or (3) make more biomass. The bioenergy required to achieve each outcome can be determined from textbook principles (FIG. 3). To evaluate whether external reducing power can enhance the formation of product, BW25113 E. coli co-expressing formate dehydrogenase and a mevalonate-producing plasmid were grown in glucose minimal media and then treated with a bolus of formate during mid-exponential growth. The application of 400 μmol formate improved mevalonate production from 0.83 g/L to 1.02 g/L, indicating that cofactor feeding can enhance target molecule yield (FIG. 7). However, formate also stimulated a considerable rise in biomass and fermentation relative to control, indicating wasteful use of bioenergy. In all, only 42.6% of formate provided was productively used to increase mevalonate biosynthesis.

As voiding the majority of externally supplied bioenergy into unproductive functions is undesirable from an economic perspective, CM15 E. coli was pivoted to because it contains gene knockouts prohibiting acetate fermentation. It was surmised that such metabolic remodeling would divert carbon flux away from fermentative metabolism and encourage mevalonate biosynthesis by increasing the amount of acetyl-CoA that is available. When this scheme is repeated by applying a 400 μmol bolus of formate to CM15 E. coli co-expressing FDH and the mevalonate-producing plasmid, the amount of formate used to boost the yield of this target molecule rose to 100%. Perfect bioenergetic efficiency is demonstrated the absence of an effect of formate upon biomass formation and a modestly inverted effect upon the fermentation profile (FIG. 7). When considered with FIG. 5, these data reveal that formate not only prevents the decarboxylative metabolism of feedstock but allows retained feedstock to be stoichiometrically converted into surplus product of interest.

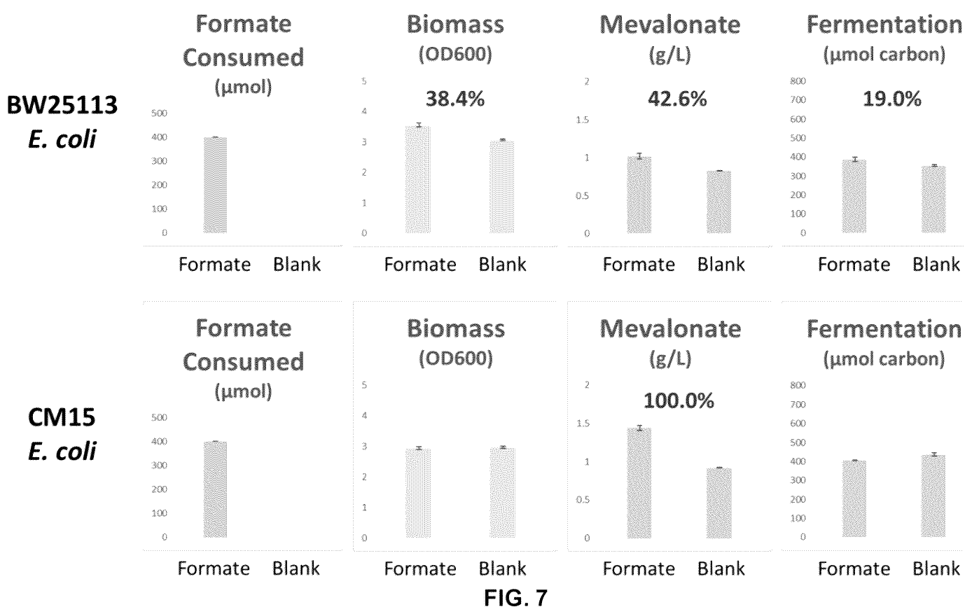

The equivalent experiment was performed using hydrogenase and H₂. Owing to an entanglement of factors including gene copy number, the type and availability of carbon sources, bacterial replication rate, and genetic background, the amount of H₂ that E. coli consumes during a benchtop experiment varies. Of the 800 μmol bolus of H₂ provided, less than one quarter of it was consumed when E. coli was cultivated in glucose minimal media, with the remainder of this gas resting unperturbed within the headspace. Nonetheless, a similar metabolic response was observed: Mevalonate production increased from 0.67 g/L to 0.74 g/L, but most of the H₂ (67.3%) was unproductively disposed to stimulate fermentative bioproducts of glucose metabolism (FIG. 8). When this experiment was replicated using the acetate fermentation knockout strain, instead of BW25113 E. coli, the H₂ usage efficiency rose to 77.4%, with only minor contributions to the fermentation profile (FIG. 8). In summary, these data demonstrate how cofactor feeding by distinct NADH donors has reproducible effects upon bacterial metabolism, that external reducing power can enhance the formation of a desired product, and that the bioenergetic efficiency of cofactor feeding can be maximized by metabolic engineering approaches.

### 3. Technoeconomic and Environmental Implications of Using NADH Donors in Industrial Microbiology

It has been demonstrated that supplemental reducing power obviates the decarboxylative metabolism of substrates and enhances the formation of products in live hosts. This core phenomenon is seen in variable media compositions, carbon sources, products, and redox donors. Furthermore, it has been shown from biochemistry principles that it is possible to determine how cells use the supplied energy (product vs. fermentation vs. biomass), and how metabolic engineering strategies can be applied to optimize the use of external reducing power to enhance product formation with efficiencies approaching 100%. Moreover, as it has been demonstrated that cofactor feeding results in titratable declines in CO₂ production, this process also potentiates the decarbonization of biomanufacturing. This is vital for meeting sustainability goals because CO₂ emissions from bioproduction can exceed those of petroleum usage due to land use changes (Merfort et al. 2013).

Is cofactor feeding financially viable? To answer this question, another is posed: What is the fair market value of proton motive force? This question, though odd, is concretely answerable. The cheapest carbohydrate that is both high in energy and digestible is sucrose. Wholesale sucrose is valued at $0.49 USD/KG, or six moles for a dollar. The complete oxidation of this much sugar through glycolysis and respiration yields 60 moles of NADH, 12 moles of FADH₂, and 24 moles of ATP/GTP—equivalent to 1536 moles of PMF. In other words, one can “buy” 1536 moles of PMF for a dollar. But this is not necessarily the cheapest possible source of cellular energy: Consider the Department of Energy's Hydrogen Earthshot program, which seeks to reduce the cost of renewable H₂ generation down to $1/KG by 2030 (Ji & Wang, 2021; Pivovar et al. 2021). At 500 moles/KG, one could instead “buy” 5000 moles of PMF for that same dollar. One may therefore use H₂ to arbitrage the financial cost of performing cellular work, as every $1 of H₂ fed to a bioreactor liberates an additional $3.25 in sugar for bioproduction. As the price of feedstock is the overwhelming consideration in the operating expenses (OpEx) of most bioprocesses (Connelly et al. 2015), the implications of cofactor feeding to improving the cost-competitiveness of industrial microbiology is astounding. As our data have revealed, this process is generalizable and quantitative. Indeed, there is very broad potential for producing diverse molecules of industrial significance. For example, it is observed that treating E. coli with H₂ grown in LB elevated intracellular succinate concentration 30-fold (FIG. 16). The U.S. Department of Energy identifies succinic acid as a priority building block chemical for its use it making 1,4-butanediol and other valorized chemicals (Werpy & Petersen, 2004). Although several companies have chased bio-succinic acid, the economic viability of these promising practices fell victim to the shale oil revolution. As succinic acid is redox imbalanced when produced from glucose (Zhu & Tang, 2017), there is opportunity for cofactor feeding to revitalize the bioproduction of this and other commodity chemicals.

Similarly, formate was effective and efficient. Using electricity from wind farms, electrocatalysis of CO₂ into formate is possible for only $0.11/KG (De Luna et al. 2019). However, after accounting for capital expenditures (CapEx) and CO₂ sourcing, the plausible value of electrocatalyzed formate is closer to $0.46/KG (Spurgeon & Kumar, 2018). Regrettably, at this price, one dollar of formate would only offer energy equivalent to 472 moles of PMF, or one third of sucrose, meaning that formate cannot arbitrage the cost of bioreactions relying upon sucrose. However, when considering which redox donor to use, there are broader technoeconomic factors at play. For example, the overwhelming cost consideration in the manufacturing of H₂ and formate is the price of electricity. The cost of renewable electricity continues to plummet and can be competitive on cost basis alone (IREA, 2018). Some wind farms are already delivering $0.04 kWh, and the Department of Energy, having already successfully met 2020 photovoltaic targets of $0.06 kWh, is now setting sights on $0.03 kWh in Sunshot 2030. Moreover, electricity supply and demand are often mismatched such that there are periods of substantial under-consumption resulting in negative electricity prices, and vice versa, potentiating the production of NADH donors at exceptionally attractive rates (Parag & Sovacool, 2016). As grid batteries remain a long way away, intermittent and off-peak energy production could instead be stored as chemical bonds (Qi et al. 2022; Angelidaki et al. 2018). Though H₂ has unparalleled energy density per unit mass, it has poor energy per unit volume, and it costs substantially more than $1/KG to bottle and pressurize the gas. Hydrogen gas is therefore a poor candidate for an energy reservoir. Unless strategies to disintermediate H₂ production and end-use are available, such as direct piping or on-site electrolysis from consistent electricity sources, this will be problematic. However, liquid formate suffers no such problem, and indeed, it could even be used as an interchangeable chemical carrier for H₂ if needs be (Singh et al. 2016; Eppinger & Huang, 2017). Unlike formate, H₂ is poorly soluble in water: Solubility can be enhanced by exploiting the hydrostatic pressures of vertical bioreactor tanks, or by stirring, though this latter strategy is not economical (Takors et al. 2018). As direct air capture and CO₂ scrubbing technologies continue to improve (Keith et al. 2018), there emerges the potential for integrating formate into the broader bioeconomy, including direct assimilation approaches as well as its valorization to methanol or methane (Yishai et al. 2016; Huang & Hu, 2018; Claassens et al. 2019; Cotton et al. 2020). It is conceivable that industrial microorganisms could be developed capable of flexibly harnessing the reducing power offered by H₂ and various C₁ molecules to arbitrage dynamic price changes in energy generation and storage.

### CONCLUSIONS

An integrated biochemical and economic framework is provided for achieving cost-advantaged biomanufacturing. The soluble hydrogenase from C. necator was developed into a versatile and modular tool for delivering reducing power into live cells. Whereas feedstocks are conventionally used to supply industrial hosts with the substrate for bioconversion and bioenergy by decarboxylative metabolism, it is demonstrated that cofactor feeding can replace feedstock as the source of bioenergy. This enhances the production of a molecule of interest in a bioenergetically efficient, quantifiable, and reproducible manner using various bioenergy donors. The declining cost of electricity combined with processivity improvements in H₂ and formate production supports an economic model wherein chemical electron donors may arbitrage the financial cost of achieving cellular work. Its applicability towards variable target molecules, media compositions, carbon sources, and NADH donors illustrates a broad potential for improving diverse biomanufacturing schemes. As articulated by the Biden administration's Executive Order 14081 and the United Nations Sustainable Development Goals, decarbonizing transportation and manufacturing through sustainable and efficient practices is vital for preserving environments and biodiversity, ensuring global energy security, and advancing socioeconomic development. The ability of our hydrogenase platform and cofactor feeding method to simultaneously suppress CO₂ production while improving the cost-competitiveness of biomanufacturing will significantly aid our desire to originate an affordable and carbon-neutral future.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A system for introducing a nucleic acid encoding a hydrogenase, and/or a dehydrogenase, into a host cell comprising: (a) an expression vector comprising hydrogenase genes, and/or dehydrogenase genes, operatively linked to a promoter; and (b) a delivery vector encoding genes to integrate the hydrogenase genes, and/or dehydrogenase genes, and the promoter into a chromosome of the host cell.

2. The system of claim 1, wherein the hydrogenase is a wild-type hydrogenase, or homologous variant thereof.

3. The system of claim 2, wherein the hydrogenase is Cupriavidus necator H16 hydrogenase, or homologous variant thereof.

4. The system of claim 1, wherein the dehydrogenase is a wild-type, dehydrogenase, or homologous variant thereof.

5. The system of claim 4, wherein the dehydrogenase is a formate dehydrogenase, methanol dehydrogenase, formaldehyde dehydrogenase, or a homologous variant thereof.

6. The system of claim 1, wherein the genes to integrate the hydrogenase genes and the promoter into a chromosome of the host cell are any recombinases listed in Table 1.

7. The system of claim 6, wherein the genes to integrate the hydrogenase genes and the promoter into a chromosome of the host cell are the Cre-Lox genes.

8. A genetically modified host cell comprising a nucleic acid comprising: (a) one or more nucleotide sequence(s) encoding a hydrogenase, dehydrogenase, or homologous thereof, each independently operatively linked to a promoter; and (b) optionally a further nucleotide sequence encoding a NAD(P) transhydrogenase operatively linked to a promoter; and (c) optionally a further nucleotide sequence encoding a nucleotide sequence of interest operatively linked to a promoter, wherein the genetically modified host cell has an increased cellular reducing power compared an unmodified host cell.

9. The genetically modified host cell of claim 8, wherein the hydrogenase is heterologous to the host cell.

10. The genetically modified host cell of claim 8, wherein the hydrogenase is a soluble hydrogenase (SH) from Cupriavidus necator H16, or a homologous variant thereof.

11. The genetically modified host cell of claim 10, wherein a 13 gene operon encoding the SH, or a homologous variant thereof, is integrated into the genome of the genetically modified host cell.

12. A method for introducing a nucleic acid encoding a heterologous hydrogenase into a host cell comprising: (a) introducing a nucleic acid comprising: (i) one or more nucleotide sequence(s) encoding a hydrogenase, dehydrogenase, or homologous thereof, each independently operatively linked to a promoter; and (ii) optionally a further nucleotide sequence encoding a NAD(P) transhydrogenase operatively linked to a promoter; and (iii) optionally a further nucleotide sequence encoding a nucleotide sequence of interest operatively linked to a promoter, into a host cell, and (b) optionally integrating the nucleic acid into a chromosome of the host cell.

13. A method for producing a compound of interest, comprising: (a) providing the host cell of claim 8; (b) increasing reducing power for the host cell; and (c) growing or culturing the host cell in a medium such that the hydrogenase and biosynthetic enzyme(s) are expressed thereby the compound of interest is produced.

14. The method of claim 13, wherein increasing reducing power for the host cell comprises providing or introducing or adding a reducing agent to the medium.

15. The method of claim 14, wherein reducing agent is an electron, hydrogen, formate, methanol, formaldehyde, and/or any compound that can donate electrons via a dehydrogenase, or a mixture thereof.

16. The method of claim 15, wherein reducing agent is a hydrogen, and the host cell expresses a hydrogenase.

17. The method of claim 15, wherein the reducing agent is a formate, and the host cell expresses a formate dehydrogenase.

18. The method of claim 14, wherein the reducing agent is a methanol, and the host cell expresses a methanol dehydrogenase.

19. The method of claim 14, wherein the reducing agent is a formaldehyde, and the host cell expresses a formaldehyde dehydrogenase.

20. The method of claim 13, wherein the compound of interest is a mevalonate, terpene, isoprenoid, carboxylic acid, lactone, trimethylpentanoic acid, 1-deoxyxylulose 5-phosphate, 1-deoxy-D-xylulose 5-phosphate (DXP), fatty acid, or derivatives thereof, alkyl lactone, lactam, isoprenyl alkanoate, 3-methyl-2-buten-1-ol, 3-methyl-3-buten-1-ol, 3-methyl-butan-1-ol, fatty acid ester, alpha-olefin, diacid, diamine, sesquiterpene, bisabolene, or oxidized aromatic amino acid.
